SEQUENCE LISTING (229 sequences)

SEQ ID NO: 1 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 117 aa, Artificial Sequence; identical to SEQ ID NO: 1
  Description of Artificial Sequence Synthetic polypeptide

SEQ ID NO: 4 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 5 - protein, 117 aa, Artificial Sequence; identical to SEQ ID NO: 1
  Description of Artificial Sequence Synthetic polypeptide

SEQ ID NO: 6 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 7 - protein, 117 aa, Artificial Sequence; identical to SEQ ID NO: 1
  Description of Artificial Sequence Synthetic polypeptide

SEQ ID NO: 8 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 117 aa, Artificial Sequence; identical to SEQ ID NO: 1
  Description of Artificial Sequence Synthetic polypeptide

SEQ ID NO: 10 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 11 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 12 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 13 - protein, 117 aa, Artificial Sequence; identical to SEQ ID NO: 1
  Description of Artificial Sequence Synthetic polypeptide

SEQ ID NO: 14 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 15 - protein, 117 aa, Artificial Sequence; identical to SEQ ID NO: 1
  Description of Artificial Sequence Synthetic polypeptide

SEQ ID NO: 16 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 17 - protein, 117 aa, Artificial Sequence; identical to SEQ ID NO: 1
  Description of Artificial Sequence Synthetic polypeptide

SEQ ID NO: 18 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 19 - protein, 117 aa, Artificial Sequence; identical to SEQ ID NO: 1
  Description of Artificial Sequence Synthetic polypeptide

SEQ ID NO: 20 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 21 - protein, 117 aa, Artificial Sequence; identical to SEQ ID NO: 1
  Description of Artificial Sequence Synthetic polypeptide

SEQ ID NO: 22 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 23 - protein, 117 aa, Artificial Sequence; identical to SEQ ID NO: 1
  Description of Artificial Sequence Synthetic polypeptide

SEQ ID NO: 24 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 25 - protein, 117 aa, Artificial Sequence; identical to SEQ ID NO: 1
  Description of Artificial Sequence Synthetic polypeptide

SEQ ID NO: 26 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 27 - protein, 117 aa, Artificial Sequence; identical to SEQ ID NO: 1
  Description of Artificial Sequence Synthetic polypeptide

SEQ ID NO: 28 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 29 - protein, 117 aa, Artificial Sequence; identical to SEQ ID NO: 1
  Description of Artificial Sequence Synthetic polypeptide

SEQ ID NO: 30 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 31 - protein, 117 aa, Artificial Sequence; identical to SEQ ID NO: 1
  Description of Artificial Sequence Synthetic polypeptide

SEQ ID NO: 32 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 33 - protein, 117 aa, Artificial Sequence; identical to SEQ ID NO: 1
  Description of Artificial Sequence Synthetic polypeptide

SEQ ID NO: 34 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 35 - protein, 117 aa, Artificial Sequence; identical to SEQ ID NO: 1
  Description of Artificial Sequence Synthetic polypeptide

SEQ ID NO: 36 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 37 - protein, 117 aa, Artificial Sequence; identical to SEQ ID NO: 1
  Description of Artificial Sequence Synthetic polypeptide

SEQ ID NO: 38 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 39 - protein, 117 aa, Artificial Sequence; identical to SEQ ID NO: 1
  Description of Artificial Sequence Synthetic polypeptide

SEQ ID NO: 40 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 41 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 42 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 43 - protein, 9 aa, Artificial Sequence
  Description of Artificial Sequence Synthetic peptide
GTFSSYAIS

SEQ ID NO: 44 - protein, 17 aa, Artificial Sequence
  Description of Artificial Sequence Synthetic peptide
GIIPIFGTANYAQKFQG

SEQ ID NO: 45 - protein, 18 aa, Artificial Sequence
  Description of Artificial Sequence Synthetic peptide
ARGDSSIRHAYYYYGMDV

SEQ ID NO: 46 - protein, 17 aa, Artificial Sequence
  Description of Artificial Sequence Synthetic peptide
KSSQSVLYSSNNKNYLA

SEQ ID NO: 47 - protein, 7 aa, Artificial Sequence
  Description of Artificial Sequence Synthetic peptide
WASTRES

SEQ ID NO: 48 - protein, 9 aa, Artificial Sequence
  Description of Artificial Sequence Synthetic peptide
QQYYSTPIT

SEQ ID NO: 49 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 50 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 51 - protein, 11 aa, Artificial Sequence
  Description of Artificial Sequence Synthetic peptide
GSISSSSYYWG

SEQ ID NO: 52 - protein, 16 aa, Artificial Sequence
  Description of Artificial Sequence Synthetic peptide
SIYYSGSTYYNPSLKS

SEQ ID NO: 53 - protein, 13 aa, Artificial Sequence
  Description of Artificial Sequence Synthetic peptide
ARGSDRFHPYFDY

SEQ ID NO: 54 - protein, 11 aa, Artificial Sequence
  Description of Artificial Sequence Synthetic peptide
RASQSVSRYLA

SEQ ID NO: 55 - protein, 7 aa, Artificial Sequence
  Description of Artificial Sequence Synthetic peptide
DASNRAT

SEQ ID NO: 56 - protein, 9 aa, Artificial Sequence
  Description of Artificial Sequence Synthetic peptide
QQFDTWPPT

SEQ ID NO: 57 - protein, 117 aa, Artificial Sequence; identical to SEQ ID NO: 1
  Description of Artificial Sequence Synthetic polypeptide

SEQ ID NO: 58 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 59 - protein, 126 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 60 - protein, 113 aa, Artificial Sequence [sequence omitted]

SEQ ID NO: 61 - protein, 126 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 62 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 63 - protein, 9 aa, Artificial Sequence
  Description of Artificial Sequence Synthetic peptide
YTFTSYYMH

SEQ ID NO: 64 - protein, 17 aa, Artificial Sequence
  Description of Artificial Sequence Synthetic peptide
IINPSGGSTSYAQKFQG

SEQ ID NO: 65 - protein, 19 aa, Artificial Sequence
  Description of Artificial Sequence Synthetic peptide
ARGAPNYGDTTHDYYYMDV

SEQ ID NO: 66 - protein, 11 aa, Artificial Sequence
  Description of Artificial Sequence Synthetic peptide
RASQSVSSNLA

SEQ ID NO: 67 - protein, 7 aa, Artificial Sequence
  Description of Artificial Sequence Synthetic peptide
GASTRAT

SEQ ID NO: 68 - protein, 9 aa, Artificial Sequence
  Description of Artificial Sequence Synthetic peptide
QQYDDWPFT

SEQ ID NO: 69 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 70 - protein, 107 aa, Artificial Sequence
  Description of Artificial Sequence Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 71 - protein, 9 aa, Artificial Sequence
  Description of Artificial Sequence Synthetic peptide
YTFTGYYMH

SEQ ID NO: 72 - protein, 17 aa, Artificial Sequence
  Description of Artificial Sequence Synthetic peptide
WINPNSGGTNYAQKFQG

SEQ ID NO: 73 - protein, 17 aa, Artificial Sequence
  Description of Artificial Sequence Synthetic peptide
ARDTGEYYDTDDHGMDV

SEQ ID NO: 74 - protein, 11 aa, Artificial Sequence
  Description of Artificial Sequence Synthetic peptide
RASQSVSSNLA

SEQ ID NO: 75 - protein, 7 aa, Artificial Sequence
  Description of Artificial Sequence Synthetic peptide
GASTRAT

SEQ ID NO: 76 - protein, 9 aa, Artificial Sequence
  Description of Artificial Sequence Synthetic peptide
QQDDYWPPT

SEQ ID NO: 77 - protein, 121 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 78 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 79 - protein, 9 aa, Artificial Sequence
  Description of Artificial Sequence Synthetic peptide
FTFSSYAMS

SEQ ID NO: 80 - protein, 17 aa, Artificial Sequence
  Description of Artificial Sequence Synthetic peptide
AISGSGGSTYYADSVKG

SEQ ID NO: 81 - protein, 14 aa, Artificial Sequence
  Description of Artificial Sequence Synthetic peptide
AKDGGYYDSGAGDY

SEQ ID NO: 82 - protein, 11 aa, Artificial Sequence
  Description of Artificial Sequence Synthetic peptide
RASQGIDSWLA

SEQ ID NO: 83 - protein, 7 aa, Artificial Sequence
  Description of Artificial Sequence Synthetic peptide
AASSLQS

SEQ ID NO: 84 - protein, 9 aa, Artificial Sequence
  Description of Artificial Sequence Synthetic peptide
QQGVSYPRT

SEQ ID NO: 85 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 86 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 87 - protein, 9 aa, Artificial Sequence
  Description of Artificial Sequence Synthetic peptide
FTFSSYSMN

SEQ ID NO: 88 - protein, 17 aa, Artificial Sequence
  Description of Artificial Sequence Synthetic peptide
SISSSSSYIYYADSVKG

SEQ ID NO: 89 - protein, 15 aa, Artificial Sequence
  Description of Artificial Sequence Synthetic peptide
ARGAPMGAAAGWFDP

SEQ ID NO: 90 - protein, 11 aa, Artificial Sequence
  Description of Artificial Sequence Synthetic peptide
RASQGISSWLA

SEQ ID NO: 91 - protein, 7 aa, Artificial Sequence
  Description of Artificial Sequence Synthetic peptide
AASSLQS

SEQ ID NO: 92 - protein, 9 aa, Artificial Sequence
  Description of Artificial Sequence Synthetic peptide
QQGVSFPRT

SEQ ID NO: 93 - protein, 125 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 94 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 95 - protein, 9 aa, Artificial Sequence
  Description of Artificial Sequence Synthetic peptide
YTFTSYYMH

SEQ ID NO: 96 - protein, 17 aa, Artificial Sequence
  Description of Artificial Sequence Synthetic peptide
IINPSGGSTSYAQKFQG

SEQ ID NO: 97 - protein, 18 aa, Artificial Sequence
  Description of Artificial Sequence Synthetic peptide
AREGAGFAYGMDYYYMDV

SEQ ID NO: 98 - protein, 11 aa, Artificial Sequence
  Description of Artificial Sequence Synthetic peptide
RASQSVSSYLA

SEQ ID NO: 99 - protein, 7 aa, Artificial Sequence
  Description of Artificial Sequence Synthetic peptide
DASNRAT

SEQ ID NO: 100 - protein, 9 aa, Artificial Sequence
  Description of Artificial Sequence Synthetic peptide
QQSDNWPFT

SEQ ID NO: 101 - protein, 121 aa, Homo sapiens
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 102 - protein, 110 aa, Homo sapiens
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 103 - protein, 115 aa, Homo sapiens
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 104 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 105 - protein, 9 aa, Artificial Sequence
  Description of Artificial Sequence Synthetic peptide
GSFSGYYWS

SEQ ID NO: 106 - protein, 16 aa, Artificial Sequence
  Description of Artificial Sequence Synthetic peptide
EIDHSGSTNYNPSLKS

SEQ ID NO: 107 - protein, 11 aa, Artificial Sequence
  Description of Artificial Sequence Synthetic peptide
ARARGPWSFDP

SEQ ID NO: 108 - protein, 9 aa, Artificial Sequence
  Description of Artificial Sequence Synthetic peptide
GTFSSYAIS

SEQ ID NO: 109 - protein, 17 aa, Artificial Sequence
  Description of Artificial Sequence Synthetic peptide
GIIPIFGTANYAQKFQG

SEQ ID NO: 110 - protein, 19 aa, Artificial Sequence
  Description of Artificial Sequence Synthetic peptide
ARRGRKASGSFYYYYGMDV

SEQ ID NO: 111 - protein, 17 aa, Artificial Sequence
  Description of Artificial Sequence Synthetic peptide
ESSQSLLNSGNQKNYLT

SEQ ID NO: 112 - protein, 7 aa, Artificial Sequence
  Description of Artificial Sequence Synthetic peptide
WASTRES

SEQ ID NO: 113 - protein, 9 aa, Artificial Sequence
  Description of Artificial Sequence Synthetic peptide
QNDYSYPYT

SEQ ID NO: 114 - protein, 117 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSS

SEQ ID NO: 115 - protein, 7 aa, Artificial Sequence
  Description of Artificial Sequence Synthetic peptide
GFTFSSY

SEQ ID NO: 116 - protein, 6 aa, Artificial Sequence
  Description of Artificial Sequence Synthetic peptide
SGSGGS

SEQ ID NO: 117 - protein, 8 aa, Artificial Sequence
  Description of Artificial Sequence Synthetic peptide
WGDGTLNP

SEQ ID NO: 118 - protein, 111 aa, Artificial Sequence
  Description of Artificial Sequence Synthetic polypeptide
QSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVL

SEQ ID NO: 119 - protein, 11 aa, Artificial Sequence
  Description of Artificial Sequence Synthetic peptide
SNDVGAYNYVS

SEQ ID NO: 120 - protein, 7 aa, Artificial Sequence
  Description of Artificial Sequence Synthetic peptide
EVNKRPS

SEQ ID NO: 121 - protein, 11 aa, Artificial Sequence
  Description of Artificial Sequence Synthetic peptide
SSFTSGLPWVV

SEQ ID NO: 122 - protein, 116 aa, Artificial Sequence
  Description of Artificial Sequence Synthetic polypeptide
EVQLVESGGGLVQPGGSLRLSCAASGFSLTSYGVHWVRQAPGKGLEWVGVIWSGGSTDYADSVKGRFTISRDNSKNTLYLQMNSLRAEDTAVYYCARNSNNGFAYWGQGTLVTVSS

SEQ ID NO: 123 - protein, 8 aa, Artificial Sequence
  Description of Artificial Sequence Synthetic peptide
SLTSYGVH

SEQ ID NO: 124 - protein, 10 aa, Artificial Sequence
  Description of Artificial Sequence Synthetic peptide
GVIWSGGSTD

SEQ ID NO: 125 - protein, 10 aa, Artificial Sequence
  Description of Artificial Sequence Synthetic peptide
ARNSNNGFAY

SEQ ID NO: 126 - protein, 107 aa, Artificial Sequence
  Description of Artificial Sequence Synthetic polypeptide
DIQMTQSPSSLSASVGDRVTITCRASQNIYSNLAWYQQKPGKAPKLLVYAAKNLQSGVPSRFSGSGSGTDFTLTISSLQPEDFATYYCQHFYDTPWTFGQGTKVEIK

SEQ ID NO: 127 - protein, 7 aa, Artificial Sequence
  Description of Artificial Sequence Synthetic peptide
NIYSNLA

SEQ ID NO: 128 - protein, 8 aa, Artificial Sequence
  Description of Artificial Sequence Synthetic peptide
LLVYAAKN

SEQ ID NO: 129 - protein, 9 aa, Artificial Sequence
  Description of Artificial Sequence Synthetic peptide
QHFYDTPWT

SEQ ID NO: 130 - protein, 123 aa, Artificial Sequence
  Description of Artificial Sequence Synthetic polypeptide
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSS

SEQ ID NO: 131 - protein, 7 aa, Artificial Sequence
  Description of Artificial Sequence Synthetic peptide
SQSAAWN

SEQ ID NO: 132 - protein, 18 aa, Artificial Sequence
  Description of Artificial Sequence Synthetic peptide
RIYYRSKWYNDYALSVKS

SEQ ID NO: 133 - protein, 11 aa, Artificial Sequence
  Description of Artificial Sequence Synthetic peptide
GEGYGREGFAI

SEQ ID NO: 134 - protein, 106 aa, Artificial Sequence
  Description of Artificial Sequence Synthetic polypeptide
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIK

SEQ ID NO: 135 - protein, 11 aa, Artificial Sequence
  Description of Artificial Sequence Synthetic peptide
RASQTISNTLA

SEQ ID NO: 136 - protein, 7 aa, Artificial Sequence
  Description of Artificial Sequence Synthetic peptide
AASNLQS

SEQ ID NO: 137 - protein, 8 aa, Artificial Sequence
  Description of Artificial Sequence Synthetic peptide
QQYLSWFT

SEQ ID NO: 138 - protein, 829 aa, Homo sapiens
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 139 - protein, 784 aa, Homo sapiens
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIEGRGERGSQRGNGGLQLARGRTRRS

SEQ ID NO: 140 - protein, 774 aa, Homo sapiens
MGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 141 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 142 - protein, 5 aa, Artificial Sequence
  Description of Artificial Sequence Synthetic peptide
GYYWS

SEQ ID NO: 143 - protein, 9 aa, Artificial Sequence
  Description of Artificial Sequence Synthetic peptide
ARGPWSFDP

SEQ ID NO: 144 - protein, 5 aa, Artificial Sequence
  Description of Artificial Sequence Synthetic peptide
SYAIS

SEQ ID NO: 145 - protein, 16 aa, Artificial Sequence
  Description of Artificial Sequence Synthetic peptide
GDSSIRHAYYYYGMDV

SEQ ID NO: 146 - protein, 7 aa, Artificial Sequence
  Description of Artificial Sequence Synthetic peptide
SSSYYWG

SEQ ID NO: 147 - protein, 11 aa, Artificial Sequence
  Description of Artificial Sequence Synthetic peptide
GSDRFHPYFDY

SEQ ID NO: 148 - protein, 5 aa, Artificial Sequence
  Description of Artificial Sequence Synthetic peptide
SYYMH

SEQ ID NO: 149 - protein, 17 aa, Artificial Sequence
  Description of Artificial Sequence Synthetic peptide
GAPNYGDTTHDYYYMDV

SEQ ID NO: 150 - protein, 5 aa, Artificial Sequence
  Description of Artificial Sequence Synthetic peptide
GYYMH

SEQ ID NO: 151 - protein, 15 aa, Artificial Sequence
  Description of Artificial Sequence Synthetic peptide
DTGEYYDTDDHGMDV

SEQ ID NO: 152 - protein, 5 aa, Artificial Sequence
  Description of Artificial Sequence Synthetic peptide
SYAMS

SEQ ID NO: 153 - protein, 12 aa, Artificial Sequence
  Description of Artificial Sequence Synthetic peptide
DGGYYDSGAGDY

SEQ ID NO: 154 - protein, 5 aa, Artificial Sequence
  Description of Artificial Sequence Synthetic peptide
SYSMN

SEQ ID NO: 155 - protein, 13 aa, Artificial Sequence
  Description of Artificial Sequence Synthetic peptide
GAPMGAAAGWFDP

SEQ ID NO: 156 - protein, 5 aa, Artificial Sequence
  Description of Artificial Sequence Synthetic peptide
SYYMH

SEQ ID NO: 157 - protein, 16 aa, Artificial Sequence
  Description of Artificial Sequence Synthetic peptide
EGAGFAYGMDYYYMDV

SEQ ID NO: 158 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 159 - protein, 5 aa, Artificial Sequence
  Description of Artificial Sequence Synthetic peptide
SYSMN

SEQ ID NO: 160 - protein, 15 aa, Artificial Sequence
  Description of Artificial Sequence Synthetic peptide
ARGAPIGAAAGWFDP

SEQ ID NO: 161 - protein, 13 aa, Artificial Sequence
  Description of Artificial Sequence Synthetic peptide
GAPIGAAAGWFDP

SEQ ID NO: 162 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 163 - protein, 5 aa, Artificial Sequence
  Description of Artificial Sequence Synthetic peptide
SYSMN

SEQ ID NO: 164 - protein, 15 aa, Artificial Sequence
  Description of Artificial Sequence Synthetic peptide
ARGAPQGAAAGWFDP

SEQ ID NO: 165 - protein, 13 aa, Artificial Sequence
  Description of Artificial Sequence Synthetic peptide
GAPQGAAAGWFDP

SEQ ID NO: 166 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 167 - protein, 5 aa, Artificial Sequence
  Description of Artificial Sequence Synthetic peptide
SYSMN

SEQ ID NO: 168 - protein, 15 aa, Artificial Sequence
  Description of Artificial Sequence Synthetic peptide
ARGAPLGAAAGWFDP

SEQ ID NO: 169 - protein, 13 aa, Artificial Sequence
  Description of Artificial Sequence Synthetic peptide
GAPLGAAAGWFDP

SEQ ID NO: 170 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 171 - protein, 5 aa, Artificial Sequence
  Description of Artificial Sequence Synthetic peptide
SYSMN

SEQ ID NO: 172 - protein, 15 aa, Artificial Sequence
  Description of Artificial Sequence Synthetic peptide
ARGAPFGAAAGWFDP

SEQ ID NO: 173 - protein, 13 aa, Artificial Sequence
  Description of Artificial Sequence Synthetic peptide
GAPFGAAAGWFDP

SEQ ID NO: 174 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 175 - protein, 5 aa, Artificial Sequence
  Description of Artificial Sequence Synthetic peptide
SYSMN

SEQ ID NO: 176 - protein, 15 aa, Artificial Sequence
  Description of Artificial Sequence Synthetic peptide
ARGAPVGAAAGWFDP

SEQ ID NO: 177 - protein, 13 aa, Artificial Sequence
  Description of Artificial Sequence Synthetic peptide
GAPVGAAAGWFDP

SEQ ID NO: 178 - protein, 122 aa, Artificial Sequence
  Description of Artificial Sequence Synthetic polypeptide
  MOD_RES (102)..(102): Met, Leu, Ile, Val, Gln, or Phe
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 179 - protein, 5 aa, Artificial Sequence
  Description of Artificial Sequence Synthetic peptide
SYSMN

SEQ ID NO: 180 - protein, 15 aa, Artificial Sequence
  Description of Artificial Sequence Synthetic peptide
  MOD_RES (6)..(6): Met, Leu, Ile, Val, Gln, or Phe
ARGAPXGAAAGWFDP

SEQ ID NO: 181 - protein, 13 aa, Artificial Sequence
  Description of Artificial Sequence Synthetic peptide
  MOD_RES (4)..(4): Met, Leu, Ile, Val, Gln, or Phe
GAPXGAAAGWFDP

SEQ ID NO: 182 - protein, 9 aa, Artificial Sequence
  Description of Artificial Sequence Synthetic peptide
GDSVSSQSA

SEQ ID NO: 183 - protein, 7 aa, Artificial Sequence
  Description of Artificial Sequence Synthetic peptide
YYRSKWY

SEQ ID NO: 184 - protein, 11 aa, Artificial Sequence
  Description of Artificial Sequence Synthetic peptide
GEGYGREGFAI

SEQ ID NO: 185 - protein, 7 aa, Artificial Sequence
  Description of Artificial Sequence Synthetic peptide
SQTISNT

SEQ ID NO: 186 - protein, 3 aa, Artificial Sequence
  Description of Artificial Sequence Synthetic peptide
AAS

SEQ ID NO: 187 - protein, 5 aa, Artificial Sequence
  Description of Artificial Sequence Synthetic peptide
YLSWF

SEQ ID NO: 188 - protein, 120 aa, Artificial Sequence
  Description of Artificial Sequence Synthetic polypeptide
QVQLQESGPELVKPGASVKMSCKASGYSFTAYNMHWVKQSHGKSLEWIGFIDPYSGIITYNQTFKGKATLTVDKSSSTAYMQLNSLTSEDSAVYYCARRGYYDGGFDYWGQGTTLTVSSS

SEQ ID NO: 189 - protein, 10 aa, Artificial Sequence
  Description of Artificial Sequence Synthetic peptide
GYSFTAYNMH

SEQ ID NO: 190 - protein, 9 aa, Artificial Sequence
  Description of Artificial Sequence Synthetic peptide
IDPYSGIIT

SEQ ID NO: 191 - protein, 10 aa, Artificial Sequence
  Description of Artificial Sequence Synthetic peptide
RGYYDGGFDY

SEQ ID NO: 192 - protein, 109 aa, Artificial Sequence
  Description of Artificial Sequence Synthetic polypeptide
DIDIQMTQTTSSLSASLGDRVTISCRASQDITNYLNWYQQKPDGTVKLLIYYTSRLHSGVPSRFSGSGSGTDYSLTISNLEQEDIATYFCQQDSKHPRTFGGGTKLEIK

SEQ ID NO: 193 - protein, 11 aa, Artificial Sequence
  Description of Artificial Sequence Synthetic peptide
RASQDITNYLN

SEQ ID NO: 194 - protein, 7 aa, Artificial Sequence
  Description of Artificial Sequence Synthetic peptide
YTSRLHS

SEQ ID NO: 195 - protein, 9 aa, Artificial Sequence
  Description of Artificial Sequence Synthetic peptide
QQDSKHPRT

SEQ ID NO: 196 - protein, 118 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCATIDTANAFGIWGQGTMVTVSS

SEQ ID NO: 197 - protein, 10 aa, Artificial Sequence
  Description of Artificial Sequence Synthetic peptide
GYTFTSYGIS

SEQ ID NO: 198 - protein, 10 aa, Artificial Sequence
  Description of Artificial Sequence Synthetic peptide
WISAYNGNTN

SEQ ID NO: 199 - protein, 9 aa, Artificial Sequence
  Description of Artificial Sequence Synthetic peptide
IDTANAFGI

SEQ ID NO: 200 - protein, 110 aa, Artificial Sequence
  Description of Artificial Sequence Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSGVVFGGGTKLTVL

SEQ ID NO: 201 - protein, 13 aa, Artificial Sequence
  Description of Artificial Sequence Synthetic peptide
SGSSSNIGNNYVS

SEQ ID NO: 202 - protein, 7 aa, Artificial Sequence
  Description of Artificial Sequence Synthetic peptide
DNNKRPS

SEQ ID NO: 203 - protein, 11 aa, Artificial Sequence
  Description of Artificial Sequence Synthetic peptide
GTWDSSLSGVV

SEQ ID NO: 204 - protein, 118 aa, Artificial Sequence
  Description of Artificial Sequence Synthetic polypeptide
EVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCAKGSGSGAFDIWGQGTMVTVSS

SEQ ID NO: 205 - protein, 10 aa, Artificial Sequence
  Description of Artificial Sequence Synthetic peptide
GYTFTGYYMH

SEQ ID NO: 206 - protein, 10 aa, Artificial Sequence
  Description of Artificial Sequence Synthetic peptide
RINPNSGGTN

SEQ ID NO: 207 - protein, 9 aa, Artificial Sequence
  Description of Artificial Sequence Synthetic peptide
GSGSGAFDI

SEQ ID NO: 208 - protein, 107 aa, Artificial Sequence
  Description of Artificial Sequence Synthetic polypeptide
AIQMTQSPSSLSASVGDRVTITCRASQSIGKYLNWYQQIPGKAPKLLIYTASTLQTGVPSRFSGSGSGTDFTLTIVSLQPEDFATYYCQQSFNTPRTFGQGTKVEIK

SEQ ID NO: 209 - protein, 11 aa, Artificial Sequence
  Description of Artificial Sequence Synthetic peptide
RASQSIGKYLN

SEQ ID NO: 210 - protein, 7 aa, Artificial Sequence
  Description of Artificial Sequence Synthetic peptide
TASTLQT

SEQ ID NO: 211 - protein, 9 aa, Artificial Sequence
  Description of Artificial Sequence Synthetic peptide
QQSFNTPRT

SEQ ID NO: 212 - protein, 118 aa, Artificial Sequence
  Description of Artificial Sequence Synthetic polypeptide
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKSSSDRTFDYWGQGTLVTVSS

SEQ ID NO: 213 - protein, 10 aa, Artificial Sequence
  Description of Artificial Sequence Synthetic peptide
GFTFSSYGMH

SEQ ID NO: 214 - protein, 10 aa, Artificial Sequence
  Description of Artificial Sequence Synthetic peptide
VISYDGSNKY

SEQ ID NO: 215 - protein, 9 aa, Artificial Sequence
  Description of Artificial Sequence Synthetic peptide
SSSDRTFDY

SEQ ID NO: 216 - protein, 112 aa, Artificial Sequence
  Description of Artificial Sequence Synthetic polypeptide
DIVMTQSPLSLPVTLGQPASISCRSSQSLVHSDGNTYLHWFQQRPGQSPRRLIYKVSNRDSGVPDRFSGSGSGTDFTLKISRVEAEDVGVYYCMQGTHWPPLFGQGTKVEIK

SEQ ID NO: 217 - protein, 16 aa, Artificial Sequence
  Description of Artificial Sequence Synthetic peptide
RSSQSLVHSDGNTYLH

SEQ ID NO: 218 - protein, 7 aa, Artificial Sequence
  Description of Artificial Sequence Synthetic peptide
KVSNRDS

SEQ ID NO: 219 - protein, 9 aa, Artificial Sequence
  Description of Artificial Sequence Synthetic peptide
MQGTHWPPL

SEQ ID NO: 220 - protein, 117 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCADTNSAKFDPWGQGTMVTVSS

SEQ ID NO: 221 - protein, 10 aa, Artificial Sequence
  Description of Artificial Sequence Synthetic peptide
GFTFSSYAMS

SEQ ID NO: 222 - protein, 10 aa, Artificial Sequence
  Description of Artificial Sequence Synthetic peptide
AISGSGGSTY

SEQ ID NO: 223 - protein, 8 aa, Artificial Sequence
  Description of Artificial Sequence Synthetic peptide
TNSAKFDP

SEQ ID NO: 224 - protein, 111 aa, Artificial Sequence
  Description of Artificial Sequence Synthetic polypeptide
QSALTQPASVSGSPGQSITISCTGTSNDVGAYNYVSWYQQHPGKAPKLILSEVNKRPSGVSNRFSGSKSGNTASLSISGLQAEDEADYYCTSYTMGSTFMLFGGGTKLTVL

SEQ ID NO: 225 - protein, 14 aa, Artificial Sequence
  Description of Artificial Sequence Synthetic peptide
TGTSNDVGAYNYVS

SEQ ID NO: 226 - protein, 11 aa, Artificial Sequence
  Description of Artificial Sequence Synthetic peptide
TSYTMGSTFML

SEQ ID NO: 227 - protein, 117 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCATIAPGRFDPWGQGTMVTVSS

SEQ ID NO: 228 - protein, 8 aa, Artificial Sequence
  Description of Artificial Sequence Synthetic peptide
IAPGRFDP

SEQ ID NO: 229 - protein, 111 aa, Artificial Sequence
  Description of Artificial Sequence Synthetic polypeptide
QSALTQPASVSGPPGQSITISCTGTSNDVGAYNYVSWYQQHPGKAPKLILSEVNKRPSGVSNRFSGSKSGNTASLSISGLQAEDEADYYCTSYTMGSTFMLFGGGTKLTVL

(19) United States
(12) Patent Application Publication
(10) Pub. No.: US 2021/0238290 A1
(43) Pub. Date: Aug. 5, 2021

(54) PROTEINS BINDING NKG2D, CD16 AND P-CADHERIN

(71) Applicant: Dragonfly Therapeutics, Inc., Waltham, MA (US)

(72) Inventors: Gregory P. Chang, Medford, MA (US); Ann F. Cheung, Lincoln, MA (US); Jinyan Du, Waltham, MA (US); Asya Grinberg, Lexington, MA (US); William Haney, Wayland, MA (US); Bradley M. Lunde, Lebanon, NH (US); Bianka Prinz, Lebanon, NH (US)

(21) Appl. No.: 17/053,558

(22) PCT Filed: May 6, 2019
(86) PCT No.: PCT/US2019/030946

§ 371 Date: Nov. 6, 2020

Related U.S. Application Data
(60) Provisional application No. 62/667,844, filed on May 7, 2018.

(51) Int. Cl. C07K 16/28; A61P 35/00

(52) U.S. Cl. CPC C07K 16/2851; C07K 16/283; A61P 35/00; C07K 2317/31; A61K 2039/505; C07K 2317/55; C07K 2317/622; C07K 2317/73; C07K 2317/92; C07K 2317/33

(57) ABSTRACT

Multi-specific binding proteins that bind NKG2D receptor, CD16, and P-cadherin, as well as pharmaceutical compositions and therapeutic methods useful for the treatment of cancer.

### CROSS-REFERENCE TO RELATED APPLICATIONS

This application claims the benefit of and priority to U.S. Provisional Patent Application No. 62/667,844, filed May 7, 2018.

### SEQUENCE LISTING

The instant application contains a Sequence Listing which has been submitted electronically in ASCII format and is hereby incorporated by reference in its entirety. Said ASCII copy, created on Apr. 30, 2019, is named DFY-054WO_SL.txt and is 152,838 bytes in size.

### FIELD OF THE INVENTION

The invention relates to multi-specific binding proteins that bind to P-cadherin (CDH3), NKG2D, and CD16.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. Other types of cancers also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, e.g., WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-γ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, natural cytotoxicity receptors (NCRs), DNAX accessory molecule 1 (DNAM1)). NK cells are also activated by the constant region of some immunoglobulins through CD16 receptors on their surface. The overall sensitivity of NK cells to activation depends on the sum of stimulatory and inhibitory signals.

P-cadherin (CDH3) is a calcium-dependent cell-cell adhesion glycoprotein composed of five extracellular cadherin repeats, a transmembrane region and a highly conserved cytoplasmic tail. It regulates several cellular homeostatic processes that participate in embryonic development and maintenance of adult tissue architecture, being important for cell differentiation, cell shape, cell polarity, growth and migration. P-cadherin is present in several adult tissues, usually co-expressed with E-cadherin, such as the basal layer of the epidermis, breast and prostate; as well as the mesothelium, ovary, cervix, hair follicle, and corneal endothelium. Up-regulation of P-cadherin expression has been reported in esophageal, pancreatic, bladder, prostate, gastric, lung, endometrial, ovarian, cervical, head and neck, and breast cancer, as well as in colorectal carcinomas, cervical adenocarcinomas, melanoma, and basocellular and squamous carcinomas of the skin.

The present invention provides certain advantages to improve treatments for the above-mentioned cancers.

### SUMMARY

The invention provides multi-specific binding proteins that bind to P-cadherin (CDH3) on cancer cells, and to the NKG2D receptor and CD16 receptor on natural killer cells. In certain embodiments, the proteins can agonize NK cells in humans, and in other species such as rodents and cynomolgus monkeys. Various aspects and embodiments of the invention are described in further detail below.

Accordingly, certain embodiments of the invention provide a protein that incorporates a first antigen-binding site that binds NKG2D; a second antigen-binding site that binds P-cadherin; and an antibody fragment crystallizable (Fc) domain, a portion thereof sufficient to bind CD16, or a third antigen-binding site that binds CD16.

The antigen-binding sites may each incorporate an antibody heavy chain variable domain and an antibody light chain variable domain (e.g., arranged as in an antibody, or fused together to from an scFv), or one or more of the antigen-binding sites may be a single domain antibody, such as a V_{H}H antibody like a camelid antibody or a V_{NAR} antibody like those found in cartilaginous fish.

In certain embodiments, the present invention provides multi-specific binding proteins that bind to the NKG2D receptor and CD16 receptor on natural killer cells, and P-cadherin on a cancer cell, wherein the NKG2D-binding site includes a heavy chain variable domain at least 90% identical to an amino acid sequence selected from: SEQ ID NO:1, SEQ ID NO:41, SEQ ID NO:49, SEQ ID NO:57, SEQ ID NO:59, SEQ ID NO:61, SEQ ID NO:69, SEQ ID NO:77, SEQ ID NO:85, SEQ ID NO:158, SEQ ID NO:162, SEQ ID NO:166, SEQ ID NO:170, SEQ ID NO:174, SEQ ID NO:178, and SEQ ID NO:93.

The first antigen-binding site, which binds to NKG2D, in certain embodiments, can incorporate a heavy chain variable domain related to SEQ ID NO:1, such as by having an amino acid sequence at least 90% (e.g., 90%, 91%, 92%, 93%, 94%, 95%, 96%, 97%, 98%, 99%, or 100%) identical to SEQ ID NO:1, and/or incorporating amino acid sequences identical to the CDR1 (SEQ ID NO:105 or SEQ ID NO:142), CDR2 (SEQ ID NO:106), and CDR3 (SEQ ID NO:107 or SEQ ID NO:143) sequences of SEQ ID NO:1. The heavy chain variable domain related to SEQ ID NO:1 can be coupled with a variety of light chain variable domains to form a NKG2D binding site. For example, the first antigen-binding site that incorporates a heavy chain variable domain related to SEQ ID NO:1 can further incorporate a light chain variable domain selected from any one of the sequences related to SEQ ID NOs:2, 4, 6, 8, 10, 12, 14, 16, 18, 20, 22, 24, 26, 28, 30, 32, 34, 36, and 40. For example, the first antigen-binding site incorporates a heavy chain variable domain with amino acid sequences at least 90% (e.g., 90%, 91%, 92%, 93%, 94%, 95%, 96%, 97%, 98%, 99%, or 100%) identical to SEQ ID NO:1 and a light chain variable domain with amino acid sequences at least 90% (e.g., 90%, 91%, 92%, 93%, 94%, 95%, 96%, 97%, 98%, 99%, or 100%) identical to any one of the sequences selected from SEQ ID NOs:2, 4, 6, 8, 10, 12, 14, 16, 18, 20, 22, 24, 26, 28, 30, 32, 34, 36, and 40.

Alternatively, in certain embodiments the first antigen-binding site can incorporate a heavy chain variable domain related to SEQ ID NO:41 and a light chain variable domain related to SEQ ID NO:42. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:41, and/or incorporate amino acid sequences identical to the CDR1 (SEQ ID NO:43 or SEQ ID NO:144), CDR2 (SEQ ID NO:44), and CDR3 (SEQ ID NO:45 or SEQ ID NO:145) sequences of SEQ ID NO:41. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:42, and/or incorporate amino acid sequences identical to the CDR1 (SEQ ID NO:46), CDR2 (SEQ ID NO:47), and CDR3 (SEQ ID NO:48) sequences of SEQ ID NO:42.

In certain embodiments, the first antigen-binding site can incorporate a heavy chain variable domain related to SEQ ID NO:49 and a light chain variable domain related to SEQ ID NO:50. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:49, and/or incorporate amino acid sequences identical to the CDR1 (SEQ ID NO:51 or SEQ ID NO:146), CDR2 (SEQ ID NO:52), and CDR3 (SEQ ID NO:53 or SEQ ID NO:147) sequences of SEQ ID NO:49. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:50, and/or incorporate amino acid sequences identical to the CDR1 (SEQ ID NO:54), CDR2 (SEQ ID NO:55), and CDR3 (SEQ ID NO:56) sequences of SEQ ID NO:50.

Alternatively, the first antigen-binding site can incorporate a heavy chain variable domain related to SEQ ID NO:57 and a light chain variable domain related to SEQ ID NO:58, such as by having amino acid sequences at least 90% (e.g., 90%, 91%, 92%, 93%, 94%, 95%, 96%, 97%, 98%, 99%, or 100%) identical to SEQ ID NO:57 and at least 90% (e.g., 90%, 91%, 92%, 93%, 94%, 95%, 96%, 97%, 98%, 99%, or 100%) identical to SEQ ID NO:58, respectively.

In another embodiment, the first antigen-binding site can incorporate a heavy chain variable domain related to SEQ ID NO:59 and a light chain variable domain related to SEQ ID NO:60. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:59, and/or incorporate amino acid sequences identical to the CDR1 (SEQ ID NO:108), CDR2 (SEQ ID NO:109), and CDR3 (SEQ ID NO:110) sequences of SEQ ID NO:59. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:60, and/or incorporate amino acid sequences identical to the CDR1 (SEQ ID NO:111), CDR2 (SEQ ID NO:112), and CDR3 (SEQ ID NO:113) sequences of SEQ ID NO:60.

In some embodiments, the first antigen-binding site can incorporate a heavy chain variable domain related to SEQ ID NO:101 and a light chain variable domain related to SEQ ID NO:102, such as by having amino acid sequences at least 90% (e.g., 90%, 91%, 92%, 93%, 94%, 95%, 96%, 97%, 98%, 99%, or 100%) identical to SEQ ID NO:101 and at least 90% (e.g., 90%, 91%, 92%, 93%, 94%, 95%, 96%, 97%, 98%, 99%, or 100%) identical to SEQ ID NO:102 respectively. In some embodiments, the first antigen-binding site can incorporate a heavy chain variable domain related to SEQ ID NO:103 and a light chain variable domain related to SEQ ID NO:104, such as by having amino acid sequences at least 90% (e.g., 90%, 91%, 92%, 93%, 94%, 95%, 96%, 97%, 98%, 99%, or 100%) identical to SEQ ID NO:103 and at least 90% (e.g., 90%, 91%, 92%, 93%, 94%, 95%, 96%, 97%, 98%, 99%, or 100%) identical to SEQ ID NO:104 respectively.

The first antigen-binding site, which binds to NKG2D, in some embodiments, can incorporate a heavy chain variable domain related to SEQ ID NO:61 and a light chain variable domain related to SEQ ID NO:62. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:61, and/or incorporate amino acid sequences identical to the CDR1 (SEQ ID NO:63 or SEQ ID NO:148), CDR2 (SEQ ID NO:64), and CDR3 (SEQ ID NO:65 or SEQ ID NO:149) sequences of SEQ ID NO:61. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:62, and/or incorporate amino acid sequences identical to the CDR1 (SEQ ID NO:66), CDR2 (SEQ ID NO:67), and CDR3 (SEQ ID NO:68) sequences of SEQ ID NO:62. In some embodiments, the first antigen-binding site can incorporate a heavy chain variable domain related to SEQ ID NO:69 and a light chain variable domain related to SEQ ID NO:70. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:69, and/or incorporate amino acid sequences identical to the CDR1 (SEQ ID NO:71 or SEQ ID NO:150), CDR2 (SEQ ID NO:72), and CDR3 (SEQ ID NO:73 or SEQ ID NO:151) sequences of SEQ ID NO:69. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:74), CDR2 (SEQ ID NO:75), and CDR3 (SEQ ID NO:76) sequences of SEQ ID NO:70.

In some embodiments, the first antigen-binding site can incorporate a heavy chain variable domain related to SEQ ID NO:77 and a light chain variable domain related to SEQ ID NO:78. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:77, and/or incorporate amino acid sequences identical to the CDR1 (SEQ ID NO:79 or SEQ ID NO:152), CDR2 (SEQ ID NO:80), and CDR3 (SEQ ID NO:81 or SEQ ID NO:153) sequences of SEQ ID NO:77. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:78, and/or incorporate amino acid sequences identical to the CDR1 (SEQ ID NO:82), CDR2 (SEQ ID NO:83), and CDR3 (SEQ ID NO:84) sequences of SEQ ID NO:78.

In some embodiments, the first antigen-binding site can incorporate a heavy chain variable domain related to SEQ ID NO:85 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:85, and/or incorporate amino acid sequences identical to the CDR1 (SEQ ID NO:87 or SEQ ID NO:154), CDR2 (SEQ ID NO:88), and CDR3 (SEQ ID NO:89 or SEQ ID NO:155) sequences of SEQ ID NO:85. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:158 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:158, and/or incorporate amino acid sequences identical to the CDR1 (SEQ ID NO:87 or SEQ ID NO:159), CDR2 (SEQ ID NO:88), and CDR3 (SEQ ID NO:160 or SEQ ID NO:161) sequences of SEQ ID NO:158. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:162 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:162, and/or incorporate amino acid sequences identical to the CDR1 (SEQ ID NO:87 or SEQ ID NO:163), CDR2 (SEQ ID NO:88), and CDR3 (SEQ ID NO:164 or SEQ ID NO:165) sequences of SEQ ID NO:162. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:166 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:166, and/or incorporate amino acid sequences identical to the CDR1 (SEQ ID NO:87 or SEQ ID NO:167), CDR2 (SEQ ID NO:88), and CDR3 (SEQ ID NO:168 or SEQ ID NO:169) sequences of SEQ ID NO:166. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:170 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:170, and/or incorporate amino acid sequences identical to the CDR1 (SEQ ID NO:87 or SEQ ID NO:171), CDR2 (SEQ ID NO:88), and CDR3 (SEQ ID NO:172 or SEQ ID NO:173) sequences of SEQ ID NO:170. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:174 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:174, and/or incorporate amino acid sequences identical to the CDR1 (SEQ ID NO:87 or SEQ ID NO:175), CDR2 (SEQ ID NO:88), and CDR3 (SEQ ID NO:176 or SEQ ID NO:177) sequences of SEQ ID NO:174. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:178 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:178, and/or incorporate amino acid sequences identical to the CDR1 (SEQ ID NO:87 or SEQ ID NO:179), CDR2 (SEQ ID NO:88), and CDR3 (SEQ ID NO:180 or SEQ ID NO:181) sequences of SEQ ID NO:178. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:93 and a light chain variable domain related to SEQ ID NO:94. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:93, and/or incorporate amino acid sequences identical to the CDR1 (SEQ ID NO:95 or SEQ ID NO:156), CDR2 (SEQ ID NO:96), and CDR3 (SEQ ID NO:97 or SEQ ID NO:157) sequences of SEQ ID NO:93. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:94, and/or incorporate amino acid sequences identical to the CDR1 (SEQ ID NO:98), CDR2 (SEQ ID NO:99), and CDR3 (SEQ ID NO:100) sequences of SEQ ID NO:94.

In certain embodiments, the second antigen-binding site can bind to P-cadherin and can optionally incorporate a heavy chain variable domain related to SEQ ID NO:114 and a light chain variable domain related to SEQ ID NO:118. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:114, and/or incorporate amino acid sequences identical to the CDR1 (SEQ ID NO:115), CDR2 (SEQ ID NO:116), and CDR3 (SEQ ID NO:117) sequences of SEQ ID NO:114. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:118, and/or incorporate amino acid sequences identical to the CDR1 (SEQ ID NO:119), CDR2 (SEQ ID NO:120), and CDR3 (SEQ ID NO:121) sequences of SEQ ID NO:118.

Alternatively, the second antigen-binding site binding to P-cadherin can optionally incorporate a heavy chain variable domain related to SEQ ID NO:122 and a light chain variable domain related to SEQ ID NO:126. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:122, and/or incorporate amino acid sequences identical to the CDR1 (SEQ ID NO:123), CDR2 (SEQ ID NO:124), and CDR3 (SEQ ID NO:125) sequences of SEQ ID NO:122. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:126, and/or incorporate amino acid sequences identical to the CDR1 (SEQ ID NO:127), CDR2 (SEQ ID NO:128), and CDR3 (SEQ ID NO:129) sequences of SEQ ID NO:126.

Alternatively, the second antigen-binding site binding to P-cadherin can optionally incorporate a heavy chain variable domain related to SEQ ID NO:130 and a light chain variable domain related to SEQ ID NO:134. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:130, and/or incorporate amino acid sequences identical to the CDR1 (SEQ ID NO:131 or SEQ ID NO:182), CDR2 (SEQ ID NO:132 or SEQ ID NO:183), and CDR3 (SEQ ID NO:133 or SEQ ID NO:184) sequences of SEQ ID NO:130. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:134, and/or incorporate amino acid sequences identical to the CDR1 (SEQ ID NO:135 or SEQ ID NO:185), CDR2 (SEQ ID NO:136 or SEQ ID NO:186), and CDR3 (SEQ ID NO:137 or SEQ ID NO:187) sequences of SEQ ID NO:134.

Alternatively, the second antigen-binding site binding to P-cadherin can optionally incorporate a heavy chain variable domain related to SEQ ID NO:188 and a light chain variable domain related to SEQ ID NO:192. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:188, and/or incorporate amino acid sequences identical to the CDR1 (SEQ ID NO:189), CDR2 (SEQ ID NO:190), and CDR3 (SEQ ID NO:191) sequences of SEQ ID NO:188. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:192, and/or incorporate amino acid sequences identical to the CDR1 (SEQ ID NO:193), CDR2 (SEQ ID NO:194), and CDR3 (SEQ ID NO:195) sequences of SEQ ID NO:192.

Alternatively, the second antigen-binding site binding to P-cadherin can optionally incorporate a heavy chain variable domain related to SEQ ID NO:196 and a light chain variable domain related to SEQ ID NO:200. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:196, and/or incorporate amino acid sequences identical to the CDR1 (SEQ ID NO:197), CDR2 (SEQ ID NO:198), and CDR3 (SEQ ID NO:199) sequences of SEQ ID NO:196. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:200, and/or incorporate amino acid sequences identical to the CDR1 (SEQ ID NO:201), CDR2 (SEQ ID NO:202), and CDR3 (SEQ ID NO:203) sequences of SEQ ID NO:200.

Alternatively, the second antigen-binding site binding to P-cadherin can optionally incorporate a heavy chain variable domain related to SEQ ID NO:204 and a light chain variable domain related to SEQ ID NO:208. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:204, and/or incorporate amino acid sequences identical to the CDR1 (SEQ ID NO:205), CDR2 (SEQ ID NO:206), and CDR3 (SEQ ID NO:207) sequences of SEQ ID NO:204. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:208, and/or incorporate amino acid sequences identical to the CDR1 (SEQ ID NO:209), CDR2 (SEQ ID NO:210), and CDR3 (SEQ ID NO:211) sequences of SEQ ID NO:208.

Alternatively, the second antigen-binding site binding to P-cadherin can optionally incorporate a heavy chain variable domain related to SEQ ID NO:212 and a light chain variable domain related to SEQ ID NO:216. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:212, and/or incorporate amino acid sequences identical to the CDR1 (SEQ ID NO:213), CDR2 (SEQ ID NO:214), and CDR3 (SEQ ID NO:215) sequences of SEQ ID NO:212. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:216, and/or incorporate amino acid sequences identical to the CDR1 (SEQ ID NO:217), CDR2 (SEQ ID NO:218), and CDR3 (SEQ ID NO:219) sequences of SEQ ID NO:216.

Alternatively, the second antigen-binding site binding to P-cadherin can optionally incorporate a heavy chain variable domain related to SEQ ID NO:220 and a light chain variable domain related to SEQ ID NO:224. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:220, and/or incorporate amino acid sequences identical to the CDR1 (SEQ ID NO:221), CDR2 (SEQ ID NO:222), and CDR3 (SEQ ID NO:223) sequences of SEQ ID NO:220. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:224, and/or incorporate amino acid sequences identical to the CDR1 (SEQ ID NO:225), CDR2 (SEQ ID NO:120), and CDR3 (SEQ ID NO:226) sequences of SEQ ID NO:224.

Alternatively, the second antigen-binding site binding to P-cadherin can optionally incorporate a heavy chain variable domain related to SEQ ID NO:227 and a light chain variable domain related to SEQ ID NO:229. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:227, and/or incorporate amino acid sequences identical to the CDR1 (SEQ ID NO:221), CDR2 (SEQ ID NO:222), and CDR3 (SEQ ID NO:228) sequences of SEQ ID NO:227. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:229, and/or incorporate amino acid sequences identical to the CDR1 (SEQ ID NO:225), CDR2 (SEQ ID NO:120), and CDR3 (SEQ ID NO:226) sequences of SEQ ID NO:229.

In certain embodiments, the second antigen-binding site can bind to P-cadherin and comprises CDR1, CDR2, and CDR3 sequences of a heavy chain variable domain and a light chain variable domain sequences selected from SEQ ID NO: 114 and 118; 122 and 126; 130 and 134; 188 and 192; 196 and 200; 204 and 208; 212 and 216; 220 and 224; and 227 and 229, respectively.

In some embodiments, the second antigen binding site incorporates a light chain variable domain having an amino acid sequence identical to the amino acid sequence of the light chain variable domain present in the first antigen binding site.

In some embodiments, the protein incorporates a portion of an antibody Fc domain sufficient to bind CD16, wherein the antibody Fc domain comprises hinge and CH2 domains, and/or amino acid sequences at least 90% identical to amino acid sequence 234-332 of a human IgG antibody.

In certain embodiments, the protein further incorporates a fourth antigen-binding site that binds to a tumor-associated antigen, which includes any antigen that is associated with cancer. For example, the fourth antigen-binding site may bind to human epidermal growth factor receptor 2 (HER2), CD20, CD33, B-cell maturation antigen (BCMA), prostate-specific membrane antigen (PSMA), delta-like canonical notch ligand 3 (DLL3), ganglioside GD2 (GD2), CD123, anoctamin-1 (Ano1), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophopblast glycoprotein (5T4), glycoprotein non-metatstatic melanoma protein B (GPNMB), folate receptor-alpha (FR-alpha), pregnancy-associated plasma protein A (PAPP-A), CD37, epithelial cell adhesion molecule (EpCAM), CD2, CD19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB1), insulin-like growth factor 1-receptor (IGF1R), human epidermal growth factor receptor 3 (HER3)/ERBB3, human epidermal growth factor receptor 4 (HER4)/ERBB4, mucin 1 (MUC1), tyrosine protein kinase MET (cMET), signaling lymphocytic activation molecule F7 (SLAMF7), prostate stem cell antigen (PSCA), MHC class I polypeptide-related sequence A (MICA), MHC class I polypeptide-related sequence B (MICB), TNF-related apoptosis inducing ligand receptor 1 (TRAILR1), TNF-related apoptosis inducing ligand receptor 2 (TRAILR2), melanoma associated antigen 3 (MAGE-A3), B-lymphocyte antigen B7.1 (B7.1), B-lymphocyte antigen B7.2 (B74.2), cytotoxic T-lymphocyte associated protein 4 (CTLA4), programmed cell death protein 1 (PD1), programmed cell dealth 1 ligand 1 (PD-L1), or CD25 antigen expressed on cancer cells.

Formulations containing any one of the proteins described herein; cells containing one or more nucleic acids expressing the proteins, and methods of enhancing tumor cell death using the proteins are also provided.

Another aspect of the invention provides a method of treating cancer in a patient. The method comprises administering to a patient in need thereof a therapeutically effective amount of the multi-specific binding proteins described herein. Cancers to be treated using P-cadherin-targeting multi-specific binding proteins include, for example, esophageal cancer, pancreatic cancer, bladder cancer, prostate cancer, gastric cancer, lung cancer, endometrial cancer, ovarian cancer, cervical cancer, head and neck cancer, breast cancer, colorectal carcinomas, cervical adenocarcinomas, melanomas, and basocellular and squamous carcinomas of the skin.

### BRIEF DESCRIPTION OF THE DRAWINGS

FIG. 1 is a representation of a heterodimeric, multi-specific binding protein. Each arm can represent either the NKG2D-binding domain or a binding domain to P-cadherin. The multi-specific binding protein further comprises an Fc domain or a portion thereof that binds to CD16. In some embodiments, the NKG2D-binding and P-cadherin binding domains can share a common light chain.

FIG. 2 is a representation of a heterodimeric, multi-specific binding protein. Either the NKG2D binding domain, or the P-cadherin binding domain can take an scFv format (right arm).

FIG. 3 is a line graph showing the binding affinity of NKG2D-binding domains (listed as clones) to human recombinant NKG2D in an ELISA assay.

FIG. 4 is a line graph showing the binding affinity of NKG2D-binding domains (listed as clones) to cynomolgus recombinant NKG2D in an ELISA assay.

FIG. 5 are line graphs demonstrating the binding affinity of NKG2D-binding domains (listed as clones) to mouse recombinant NKG2D in an ELISA assay.

FIG. 6 is a bar graph showing the binding of NKG2D-binding domains (listed as clones) to EL4 cells expressing human NKG2D, measured by flow cytometry as mean fluorescence intensity (MFI) fold-over-background (FOB).

FIG. 7 is a bar graph showing the binding of NKG2D-binding domains (listed as clones) to EL4 cells expressing mouse NKG2D, measured by flow cytometry as mean fluorescence intensity (MFI) fold-over-background (FOB).

FIG. 8 is a line graph showing the specific binding affinity of NKG2D-binding domains (listed as clones) for recombinant human NKG2D-Fc in a competitive binding assay with NKG2D's natural ligand, ULBP-6.

FIG. 9 is a line graph showing the binding affinity of NKG2D-binding domains (listed as clones) for recombinant human NKG2D-Fc in a competitive binding assay with NKG2D's natural ligand, MICA.

FIG. 10 is a line graph showing the binding affinity of NKG2D-binding domains (listed as clones) for recombinant mouse NKG2D-Fc in a competitive binding assay with NKG2D's natural ligand, Rae-1 delta.

FIG. 11 is a bar graph showing activation of cells expressing human NKG2D-CD3 zeta fusion proteins by NKG2D-binding domains (listed as clones) as measured by flow cytometry and quantified as the percentage of TNF-α positive cells.

FIG. 12 is a bar graph showing activation of cells expressing mouse NKG2D-CD3 zeta fusion proteins by NKG2D-binding domains (listed as clones) as measured by flow cytometry and quantified as the percentage of TNF-α positive cells.

FIG. 13 is a bar graph showing activation of human NK cells by NKG2D-binding domains (listed as clones) as measured by flow cytometry and quantified as the percentage of IFNγ⁺/CD107a⁺ cells.

FIG. 14 is a bar graph showing activation of human NK cells by NKG2D-binding domains (listed as clones) as measured by flow cytometry and quantified as the percentage of IFNγ⁺/CD107a⁺ cells.

FIG. 15 is a bar graph showing activation of mouse NK cells by NKG2D-binding domains (listed as clones) as measured by flow cytometry and quantified as the percentage of IFNγ⁺/CD107a⁺ cells.

FIG. 16 is a bar graph showing activation of mouse NK cells by NKG2D-binding domains (listed as clones) as measured by flow cytometry and quantified as the percentage of IFNγ⁺/CD107a⁺ cells.

FIG. 17 is a bar graph showing the cytotoxic effect of NKG2D-binding domains (listed as clones) on THP-1 tumor cells as measured using a Perkin Elmer DELFIA® Cytotoxicity kit assay.

FIG. 18 is a bar graph showing the melting temperature of NKG2D-binding domains (listed as clones) measured by differential scanning fluorimetry.

FIGS. 19A-19C are bar graphs showing synergistic activation of NK cells by CD16 and NKG2D binding as measured by flow cytometry and quantified as the percentage of positive cells for NK activation markers. FIG. 19A shows the percentage of CD107a⁺ cells 4 hours post-treatment with plate-bound anti-CD16 monoclonal antibody alone, anti-NKG2D antibody alone, or anti-CD16 antibody in combination with anti-NKG2D antibody. FIG. 19B shows the percentage of IFNγ⁺ cells 4 hours post-treatment with plate-bound anti-CD16 monoclonal antibody alone, anti-NKG2D antibody alone, or anti-CD16 antibody in combination with anti-NKG2D antibody. FIG. 19C shows the percentage of CD107a⁺/IFNγ⁺ cells 4 hours post-treatment with plate-bound anti-CD16 monoclonal antibody alone, anti-NKG2D antibody alone, or anti-CD16 antibody in combination with anti-NKG2D antibody. Graphs indicate the mean (n=2) ±SD. Data are representative of five independent experiments using five different healthy donors.

FIG. 20 is a representative illustration of a multi-specific binding protein in a Triomab form.

FIG. 21 is a representative illustration of a multi-specific binding protein in a KiH Common Light Chain (LC) form.

FIG. 22 is a representative illustration of a multi-specific binding protein in a dual-variable domain immunoglobulin (DVD-Ig™) form.

FIG. 23 is a representative illustration of a multi-specific binding protein in an Orthogonal Fab interface (Ortho-Fab) form.

FIG. 24 is a representative illustration of a multi-specific binding protein in a 2-in-1 Ig form.

FIG. 25 is a representative illustration of a multi-specific binding protein in an electrostatic-steering (ES) form.

FIG. 26 is a representative illustration of a multi-specific binding protein in a controlled Fab-Arm Exchange (cFAE) form.

FIG. 27 is a representative illustration of a multi-specific binding protein in a strand-exchange engineered domain (SEED) body form.

FIG. 28 is a representative illustration of a multi-specific binding protein in a LuZ-Y form.

FIG. 29 is a representative illustration of a multi-specific binding protein in a Cov-X-Body form.

FIGS. 30A and 30B are representative illustrations of a multi-specific binding protein in a d-Body form. FIG. 30A is an exemplary representative illustration of one form of a d-Body; FIG. 30B is an exemplary representative illustration of another d-Body.

FIG. 31 is a representative illustration of a multi-specific binding protein in a one-arm single chain (OAsc)-Fab form.

FIG. 32 is a representative illustration of a multi-specific binding protein in a DuetMab form.

FIG. 33 is a representative illustration of a multi-specific binding protein in a CrossmAb form.

FIG. 34 is a representative illustration of a multi-specific binding protein in a Fit-Ig form.

FIGS. 35A-35C are line graphs showing the binding affinity of anti-P-cadherin monoclonal antibodies (D153 wt mAb) and anti-P-cadherin multi-specific binding proteins (D153 DB) for P-cadherin-expressing human non-small cell lung cancer (H1975; FIG. 35A), human colorectal cancer (HCT116; FIG. 35B), and human tongue squamous cell carcinoma (CAL27; FIG. 35C) cells.

FIGS. 36A-36C are line graphs showing the binding affinity of anti-P-cadherin monoclonal antibodies (TSP7 wt mAb) and anti-P-cadherin multi-specific binding proteins (TSP7 DB) for P-cadherin-expressing human non-small cell lung cancer (H1975; FIG. 36A), human colorectal cancer (HCT116; FIG. 36B), and human tongue squamous cell carcinoma (CAL27; FIG. 36C) cells.

FIGS. 37A-37D are line graphs showing cytotoxic activity of KHYG1-CD16V human NK cells stimulated with anti-P-cadherin multi-specific binding proteins against human cancer cells. FIG. 37A is a line graph showing D153 DB multi-specific binding protein stimulation of KHYG1-CD16V-mediated cytotoxicity against H1975 cancer cells.

FIG. 37B is a line graph showing TSP7 DB multi-specific binding protein stimulation of KHYG1-CD16V-mediated cytotoxicity against H1975 cancer cells. FIG. 37C is a line graph showing D153 DB multi-specific binding protein stimulation of KHYG1-CD16V-mediated cytotoxicity against CAL27 cancer cells. FIG. 37D is a line graph showing TSP7 DB multi-specific binding protein stimulation of KHYG1-CD16V-mediated cytotoxicity against CAL27 cancer cells.

FIGS. 38A-38C are line graphs showing cytotoxic activity against P-cadherin-expressing H1975 cancer cells of primary human NK cells isolated from three separate donors (Donors 47897, 59165, and 48892; FIGS. 38A, 38B and 38C, respectively) and stimulated with multi-specific binding proteins (D153 DB) or monoclonal antibodies (D153 mAb).

FIGS. 39A and 39B are line graphs showing cytotoxic activity against P-cadherin-expressing DU145 cells of primary human NK cells isolated from two separate donors (Donors 47897 and 45054; FIGS. 39A and 39B, respectively) and stimulated with multi-specific binding proteins (D153 DB) or monoclonal antibodies (D153 mAb).

FIGS. 40A and 40B are line graphs showing cytotoxic activity against P-cadherin-expressing H1650 cancer cells of primary human NK cells isolated from two separate donors (Donors 47897 and 45054; FIGS. 40A and 40B, respectively) and stimulated with multi-specific binding proteins (D153 DB) or monoclonal antibodies (D153 mAb).

FIGS. 41A and 41B are line graphs showing cytotoxic activity against P-cadherin-expressing CAL27 (FIG. 41A) and HCC1954 (FIG. 41B) cancer cells of primary human NK cells isolated from two separate donors (Donors 59165 and 47897; FIGS. 41A and 41B, respectively) and stimulated with multi-specific binding proteins (D153 DB) or monoclonal antibodies (D153 mAb).

FIGS. 42A-42C are line graphs showing cytotoxic activity against P-cadherin-expressing H1975 (FIG. 42A), H1650 (FIG. 42B), and HCC1954 (FIG. 42C) cancer cells of donor-isolated primary human NK cells stimulated with multi-specific binding proteins (TSP7 DB) or monoclonal antibodies (TSP7 mAb).

### DETAILED DESCRIPTION

The invention provides multi-specific binding proteins that bind P-cadherin (CDH3) on cancer cells, and the NKG2D receptor and CD16 receptor on natural killer cells. In certain embodiments, the multi-specific binding proteins further include an additional antigen-binding site that binds a tumor-associated antigen.

The invention also provides pharmaceutical compositions comprising such multi-specific binding proteins, and therapeutic methods using such multi-specific binding proteins and pharmaceutical compositions, for purposes such as treating cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor associated antigen” as used herein means any antigen including but not limited to a protein, glycoprotein, ganglioside, carbohydrate, or lipid that is associated with cancer. Such antigen can be expressed on malignant cells or in the tumor microenvironment such as on tumor-associated blood vessels, extracellular matrix, mesenchymal stroma, or immune infiltrates.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Remington's Pharmaceutical Sciences, 15th Ed., Mack Publishing Co., Easton, Pa. (1975).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The invention provides multi-specific binding proteins that bind to the NKG2D receptor and CD16 receptor on natural killer cells, and P-cadherin on cancer cells. The multi-specific binding proteins are useful in the pharmaceutical compositions and therapeutic methods described herein. Binding of the multi-specific binding proteins to the NKG2D receptor and CD16 receptor on a natural killer cell enhances the activity of the natural killer cell toward destruction of tumor cells expressing P-cadherin antigen. Binding of the multi-specific binding proteins to P-cadherin-expressing cells brings the cancer cells into proximity with the natural killer cells, which facilitates direct and indirect destruction of the cancer cells by the natural killer cells. Further description of some exemplary multi-specific binding proteins is provided below.

The first component of the multi-specific binding proteins binds to NKG2D receptor-expressing cells, which can include but are not limited to NK cells, γδ T cells and CD8⁺ αβ T cells. Upon NKG2D binding, the multi-specific binding proteins may block natural ligands, such as ULBP6 and MICA, from binding to NKG2D and activating NKG2D receptors.

In certain embodiments, the second component of the multi-specific binding proteins binds to P-cadherin-expressing cells. P-cadherin-expressing cells may be found for example in, but not limited to, esophageal cancer, pancreatic cancer, bladder cancer, prostate cancer, gastric cancer, lung cancer, endometrial cancer, ovarian cancer, cervical cancer, head and neck cancer, breast cancer, colorectal carcinomas, cervical adenocarcinomas, melanomas, and basocellular and squamous cell carcinomas of the skin

The third component for the multi-specific binding proteins binds to cells expressing CD16, an Fc receptor on the surface of leukocytes including natural killer cells, macrophages, neutrophils, eosinophils, mast cells, and follicular dendritic cells.

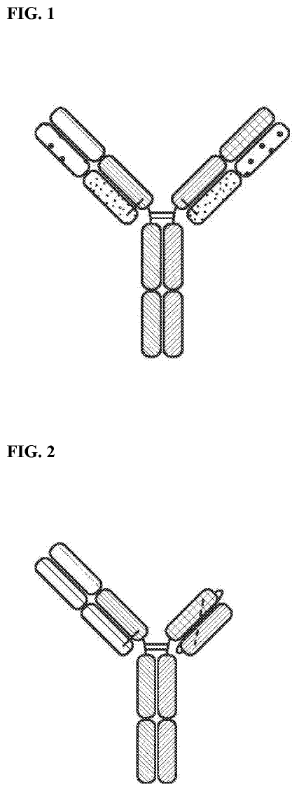

The multi-specific binding proteins described herein can take various formats. For example, one format is a heterodimeric, multi-specific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and a second light chain constant domain. In certain embodiments, the second immunoglobulin light chain, together with the second immunoglobulin heavy chain, forms an antigen-binding site that binds P-cadherin. The first Fc domain and second Fc domain together are able to bind to CD16 (FIG. 1). In some embodiments, the first immunoglobulin light chain is identical to the second immunoglobulin light chain.

Another exemplary format involves a heterodimeric, multi-specific antibody including a first immunoglobulin heavy chain, a second immunoglobulin heavy chain and an immunoglobulin light chain (FIG. 2). The first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind P-cadherin. The second immunoglobulin heavy chain includes a second Fc (hinge-CH2-CH3) domain, a second heavy chain variable domain and optionally a CH1 heavy chain domain. The immunoglobulin light chain includes a light chain variable domain and a light chain constant domain. The second immunoglobulin heavy chain pairs with the immunoglobulin light chain and binds to NKG2D or binds P-cadherin, The first Fc domain and the second Fc domain together are able to bind to CD16 (FIG. 2).

One or more additional binding motifs may be fused to the C-terminus of the constant region CH3 domain, optionally via a linker sequence. In certain embodiments, the antigen-binding site could be a single-chain or disulfide-stabilized variable region (scFv) or could form a tetravalent or trivalent molecule.

In some embodiments, the multi-specific binding protein is in a Triomab form, which is a trifunctional, bispecific antibody that maintains an IgG-like shape (e.g., the multi-specific binding protein represented in FIG. 20). This chimeric bispecific antibody comprises of two half antibodies, each with one light and one heavy chain, that originate from two parental antibodies. The Triomab form may be a heterodimer, comprising of ½ of a rat antibody and ½ of a mouse antibody.

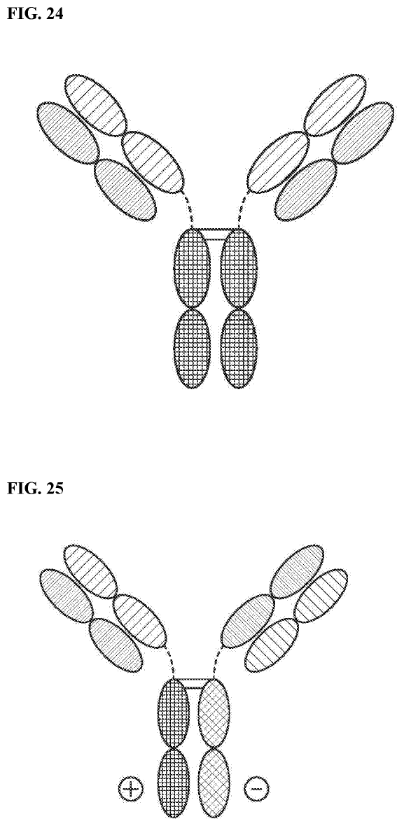

In some embodiments, the multi-specific binding protein is a KiH Common Light Chain (LC) form, which incorporates the knobs-into-holes (KiH) technology (e.g., the multi-specific binding protein represented in FIG. 21). The KiH Common LC form is a heterodimer comprising a Fab which binds to a first target, a Fab which binds to a second target, and an Fc domain stabilized by heterodimerization mutations. The two Fabs each comprise a heavy chain and light chain, wherein the heavy chain of each Fab differs from the other, and the light chain that pairs with each respective heavy chain is common to both Fabs.

The KiH technology involves engineering CH3 domains to create either a “knob” or a “hole” in each heavy chain to promote heterodimerization. Introduction of a “knob” in one CH3 domain (CH3A) comprises substitution of a small residue with a bulky one (e.g., T366W_{CH3A} in EU numbering). To accommodate the “knob,” a complementary “hole” surface is introduced on the other CH3 domain (CH3B) by replacing the closest neighboring residues to the knob with smaller ones (e.g., T366S/L368A/Y407V_{CH3B}). The “hole” mutation was optimized by structure-guided phage library screening (Atwell S., et al. (1997) J. Mol. Biol.; 270(1):26-35). X-ray crystal structures of KiH Fc variants (Elliott J. M., et al. (2014) J. Mol. Biol.; 426(9):1947-57; Mimoto F. et al. (2014) Mol. Immunol; 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in a dual-variable domain immunoglobulin (DVD-Ig™) form, which is a tetravalent IgG-like structure comprising the target-binding domains of two monoclonal antibodies and flexible naturally occurring linkers (e.g., FIG. 22). The DVD-Ig™ form is homodimeric comprising a variable domain targeting antigen 2 fused to the N-terminus of a Fab variable domain targeting antigen 1. The representative multi-specific binding protein shown in FIG. 22 comprises an unmodified Fc.

In some embodiments, the multi-specific binding protein is in an Orthogonal Fab interface (Ortho-Fab) form (e.g., the multi-specific binding protein represented in FIG. 23). In the Ortho-Fab IgG approach (Lewis S. M., et al. (2014) Nat. Biotechnol.; 32(2):191-8), structure-based regional design introduces complementary mutations at the LC and HCVH-CH1 interface in only one Fab, without any changes being made to the other Fab.

In some embodiments, the multi-specific binding protein is in a 2-in-1 Ig form (e.g., the multi-specific binding protein represented in FIG. 24).

In some embodiments, the multi-specific binding protein is an electrostatic-steering (ES) form, which is a heterodimer comprising two different Fabs binding to targets 1 and target 2, and an Fc domain (e.g., the multi-specific binding protein represented in FIG. 25). Heterodimerization is ensured by electrostatic steering mutations in the Fc domain.

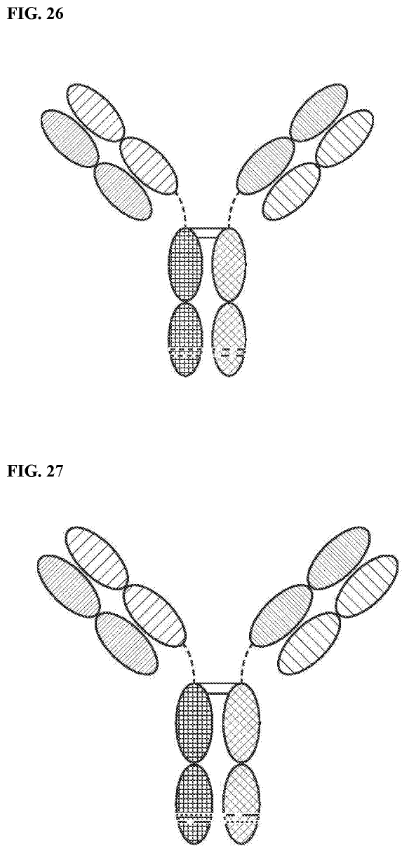

In some embodiments, the multi-specific binding protein is in a controlled Fab-Arm Exchange (cFAE) form (e.g., the multi-specific binding protein represented in FIG. 26). The cFAE form is a bispecific heterodimer comprising two different Fabs binding to targets 1 and 2, wherein a LC-HC pair (half-molecule) has been swapped with a LC-HC pair from another molecule. Heterodimerization is ensured by mutations in the Fc.

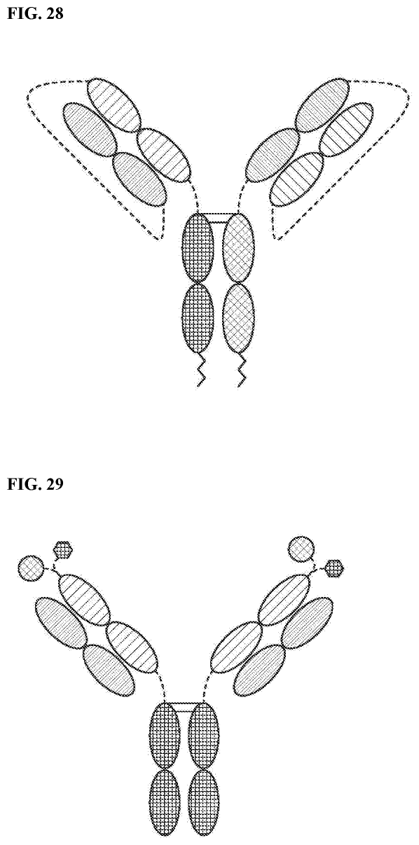

In some embodiments, the multi-specific binding protein is in a strand-exchange engineered domain (SEED) body form (e.g., the multi-specific binding protein represented in FIG. 27). The SEED platform was designed to generate asymmetric and bispecific antibody-like molecules in order to expand the therapeutic applications of natural antibodies. This protein engineering platform is based on exchanging structurally related sequences of immunoglobulin classes within the conserved CH3 domains (e.g., alternating segments of IgA and IgG CH3 domain sequences). The SEED design allows efficient generation of heterodimers, while disfavoring homodimerization of SEED CH3 domains. (Muda M. et al. (2011) Protein Eng. Des. Sel.; 24(5):447-54).

In some embodiments, the multi-specific binding protein is in a LuZ-Y form (e.g., the multi-specific binding protein represented in FIG. 28). The LuZ-Y form is a heterodimer comprising two different scFabs binding to targets 1 and 2, fused to an Fc domain. Heterodimerization is ensured through the introduction of leucine zipper motifs fused to the C-terminus of the Fc domain (Wranik, B J. et al. (2012) J. Biol. Chem.; 287:43331-9).

In some embodiments, the multi-specific binding protein is in a Cov-X-Body form (e.g., the multi-specific binding protein represented in FIG. 29). Bispecific CovX-Bodies comprise a scaffold antibody having a pharmacophore peptide heterodimer covalently linked to each Fab arm, wherein one molecule of the peptide heterodimer binds to a first target and the other molecule of the peptide heterodimer binds to a second target, and wherein the two molecules are joined by an azetidinone linker. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V. R. et al. (2010) PNAS; 107(52); 22611-22616).

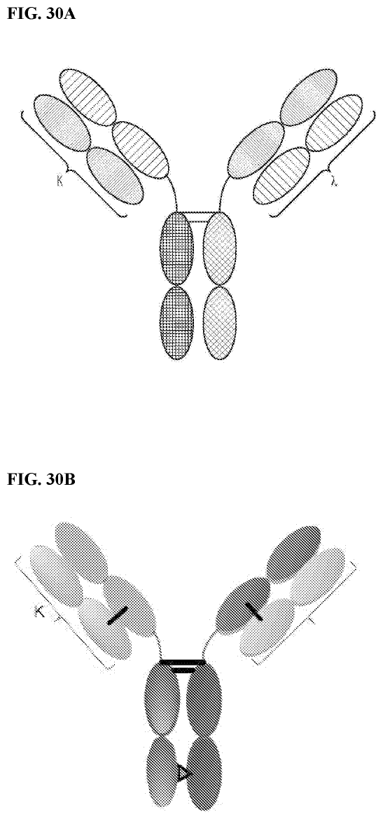

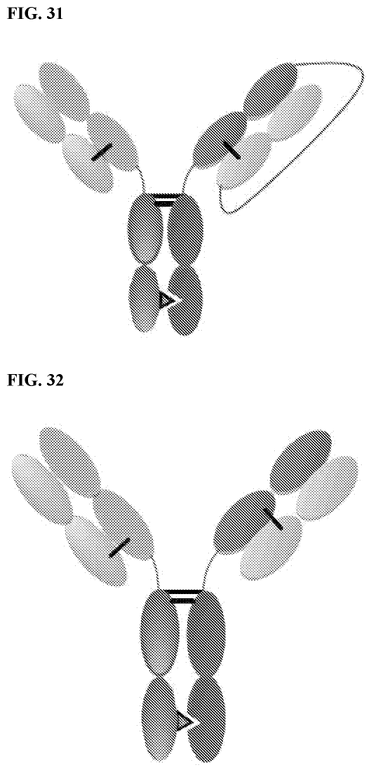

In some embodiments, the multi-specific binding protein is in a d-Body form, which is a heterodimer comprising two different Fabs fused to Fc domains stabilized by heterodimerization mutations (e.g., the multi-specific binding protein represented in FIG. 30). A first Fab binding target 1 comprises a kappa LC, and a second Fab binding target 2 comprises a lambda LC. FIG. 30A is an exemplary representation of one form of a K-Body; FIG. 30B is an exemplary representation of another d-Body.

In some embodiments, the multi-specific binding protein is in a one-arm single chain (OAsc)-Fab form (e.g., the multi-specific binding protein represented in FIG. 31). The OAsc-Fab form is a heterodimer that includes a Fab binding to target 1 and an scFab binding to target 2 fused to an Fc domain. Heterodimerization is ensured by mutations in the Fc domain.

In some embodiments, the multi-specific binding protein is in a DuetMab form (e.g., the multi-specific binding protein represented in FIG. 32). The DuetMab form is a heterodimer comprising two different Fabs binding to targets 1 and 2, and an Fc domain stabilized by heterodimerization mutations. The two different Fabs comprise different S—S bridges that ensure correct LC and HC pairing.

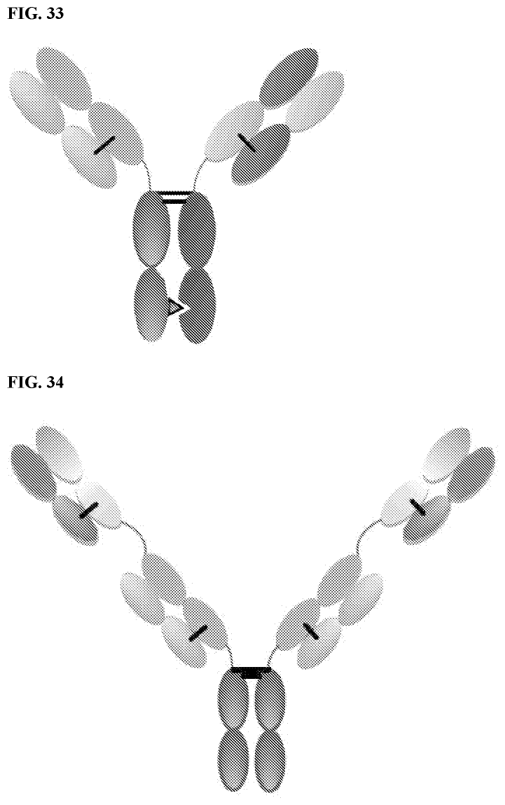

In some embodiments, the multi-specific binding protein is in a CrossmAb form (e.g., the multi-specific binding protein represented in FIG. 33). The CrossmAb form is a heterodimer comprising two different Fabs binding to targets 1 and 2, and an Fc domain stabilized by heterodimerization mutations. CL and CH1 domains and VH and VL domains are switched, e.g., CH1 is fused in-line with VL, while CL is fused in-line with VH.

In some embodiments, the multi-specific binding protein is in a Fit-Ig form (e.g., the multi-specific binding protein represented in FIG. 34). The Fit-Ig form is a homodimer comprising a Fab binding to target 2 fused to the N-terminus of the HC of a Fab that binds to target 1. The representative multi-specific binding protein of FIG. 34 comprises an unmodified Fc domain.

Table 1 lists peptide sequences of heavy chain variable domains and light chain variable domains that, in combination, can bind to NKG2D. Unless indicated otherwise, the CDR sequences provided in Table 1 are determined under Kabat. The NKG2D binding domains can vary in their binding affinity to NKG2D, nevertheless, they all activate human NKG2D and NK cells.

TABLE 1

| | Heavy chain variable region | Light chain variable region |
|---|---|---|
| Clones | amino acid sequence | amino acid sequence |
| ADI- | QVQLQQWGAGLLKPSETLSL | DIQMTQSPSTLSASVGDRVTITCRA |
| 27705 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYNSYPITFG |
| | SSVTAADTAVYYCARARGP | GGTKVEIK (SEQ ID NO: 2) |
| | WSFDPWGQGTLVTVSS | |
| | (SEQ ID NO: 1) | |
| | CDR1: GSFSGYYWS (non- | |
| | Kabat) (SEQ ID NO: 105) or | |
| | GYYWS (SEQ ID NO: 142) | |
| | CDR2: EIDHSGSTNYNPSLKS | |
| | (SEQ ID NO: 106) | |
| | CDR3: ARARGPWSFDP (non- | |
| | Kabat) (SEQ ID NO: 107) or | |
| | ARGPWSFDP (SEQ ID NO: 143) | |
| ADI- | QVQLQQWGAGLLKPSETLSL | EIVLTQSPGTLSLSPGERATLSCRA |
| 27724 | TCAVYGGSFSGYYWSWIRQP | SQSVSSSYLAWYQQKPGQAPRLLI |
| | PGKGLEWIGEIDHSGSTNYNP | YGASSRATGIPDRFSGSGSGTDFTL |
| | SLKSRVTISVDTSKNQFSLKL | TISRLEPEDFAVYYCQQYGSSPITF |
| | SSVTAADTAVYYCARARGP | GGGTKVEIK (SEQ ID NO: 4) |
| | WSFDPWGQGTLVTVSS | |
| | (SEQ ID NO: 3) | |
| ADI- | QVQLQQWGAGLLKPSETLSL | DIQMTQSPSTLSASVGDRVTITCRA |
| 27740 | TCAVYGGSFSGYYWSWIRQP | SQSIGSWLAWYQQKPGKAPKLLIY |
| (A40) | PGKGLEWIGEIDHSGSTNYNP | KASSLESGVPSRFSGSGSGTEFTLTI |
| | SLKSRVTISVDTSKNQFSLKL | SSLQPDDFATYYCQQYHSFYTFGG |
| | SSVTAADTAVYYCARARGP | GTKVEIK (SEQ ID NO: 6) |
| | WSFDPWGQGTLVTVSS | |
| | (SEQ ID NO: 5) | |
| ADI- | QVQLQQWGAGLLKPSETLSL | DIQMTQSPSTLSASVGDRVTITCRA |
| 27741 | TCAVYGGSFSGYYWSWIRQP | SQSIGSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQSNSYYTFGG |
| | SSVTAADTAVYYCARARGP | GTKVEIK (SEQ ID NO: 8) |
| | WSFDPWGQGTLVTVSS | |
| | (SEQ ID NO: 7) | |
| ADI- | QVQLQQWGAGLLKPSETLSL | DIQMTQSPSTLSASVGDRVTITCRA |
| 27743 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYNSYPTFGG |
| | SSVTAADTAVYYCARARGP | GTKVEIK (SEQ ID NO: 10) |
| | WSFDPWGQGTLVTVSS | |
| | (SEQ ID NO: 9) | |
| ADI- | QVQLQQWGAGLLKPSETLSL | ELQMTQSPSSLSASVGDRVTITCRT |
| 28153 | TCAVYGGSFSGYYWSWIRQP | SQSISSYLNWYQQKPGQPPKLLIY |
| | PGKGLEWIGEIDHSGSTNYNP | WASTRESGVPDRFSGSGSGTDFTL |
| | SLKSRVTISVDTSKNQFSLKL | TISSLQPEDSATYYCQQSYDIPYTF |
| | SSVTAADTAVYYCARARGP | GQGTKLEIK (SEQ ID NO: 12) |
| | WGFDPWGQGTLVTVSS | |
| | (SEQ ID NO: 11) | |
| ADI- | QVQLQQWGAGLLKPSETLSL | DIQMTQSPSTLSASVGDRVTITCRA |
| 28226 | TCAVYGGSFSGYYWSWIRQP | SQSISSWLAWYQQKPGKAPKLLIY |
| (C26) | PGKGLEWIGEIDHSGSTNYNP | KASSLESGVPSRFSGSGSGTEFTLTI |
| | SLKSRVTISVDTSKNQFSLKL | SSLQPDDFATYYCQQYGSFPITFGG |
| | SSVTAADTAVYYCARARGP | GTKVEIK (SEQ ID NO: 14) |
| | WSFDPWGQGTLVTVSS | |
| | (SEQ ID NO: 13) | |
| ADI- | QVQLQQWGAGLLKPSETLSL | DIQMTQSPSTLSASVGDRVTITCRA |
| 28154 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTDFTLT |
| | SLKSRVTISVDTSKNQFSLKL | ISSLQPDDFATYYCQQSKEVPWTF |
| | SSVTAADTAVYYCARARGP | GQGTKVEIK (SEQ ID NO: 16) |
| | WSFDPWGQGTLVTVSS | |
| | (SEQ ID NO: 15) | |
| ADI- | QVQLQQWGAGLLKPSETLSL | DIQMTQSPSTLSASVGDRVTITCRA |
| 29399 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYNSFPTFGG |
| | SSVTAADTAVYYCARARGP | GTKVEIK (SEQ ID NO: 18) |
| | WSFDPWGQGTLVTVSS | |
| | (SEQ ID NO: 17) | |
| ADI- | QVQLQQWGAGLLKPSETLSL | DIQMTQSPSTLSASVGDRVTITCRA |
| 29401 | TCAVYGGSFSGYYWSWIRQP | SQSIGSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYDIYPTFGG |
| | SSVTAADTAVYYCARARGP | GTKVEIK (SEQ ID NO: 20) |
| | WSFDPWGQGTLVTVSS | |
| | (SEQ ID NO: 19) | |
| ADI- | QVQLQQWGAGLLKPSETLSL | DIQMTQSPSTLSASVGDRVTITCRA |
| 29403 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYDSYPTFGG |
| | SSVTAADTAVYYCARARGP | GTKVEIK (SEQ ID NO: 22) |
| | WSFDPWGQGTLVTVSS | |
| | (SEQ ID NO: 21) | |
| ADI- | QVQLQQWGAGLLKPSETLSL | DIQMTQSPSTLSASVGDRVTITCRA |
| 29405 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYGSFPTFGG |
| | SSVTAADTAVYYCARARGP | GTKVEIK (SEQ ID NO: 24) |
| | WSFDPWGQGTLVTVSS | |
| | (SEQ ID NO: 23) | |
| ADI- | QVQLQQWGAGLLKPSETLSL | DIQMTQSPSTLSASVGDRVTITCRA |
| 29407 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYQSFPTFGG |
| | SSVTAADTAVYYCARARGP | GTKVEIK (SEQ ID NO: 26) |
| | WSFDPWGQGTLVTVSS | |
| | (SEQ ID NO: 25) | |
| ADI- | QVQLQQWGAGLLKPSETLSL | DIQMTQSPSTLSASVGDRVTITCRA |
| 29419 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYSSFSTFGG |
| | SSVTAADTAVYYCARARGP | GTKVEIK (SEQ ID NO: 28) |
| | WSFDPWGQGTLVTVSS | |
| | (SEQ ID NO: 27) | |
| ADI- | QVQLQQWGAGLLKPSETLSL | DIQMTQSPSTLSASVGDRVTITCRA |
| 29421 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYESYSTFGG |
| | SSVTAADTAVYYCARARGP | GTKVEIK (SEQ ID NO: 30) |
| | WSFDPWGQGTLVTVSS | |
| | (SEQ ID NO: 29) | |
| ADI- | QVQLQQWGAGLLKPSETLSL | DIQMTQSPSTLSASVGDRVTITCRA |
| 29424 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYDSFITFGG |
| | SSVTAADTAVYYCARARGP | GTKVEIK (SEQ ID NO: 32) |
| | WSFDPWGQGTLVTVSS | |
| | (SEQ ID NO: 31) | |
| ADI- | QVQLQQWGAGLLKPSETLSL | DIQMTQSPSTLSASVGDRVTITCRA |
| 29425 | TCAVYGGSFSGYYWSWIRQP | SQSISSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYQSYPTFGG |
| | SSVTAADTAVYYCARARGP | GTKVEIK (SEQ ID NO: 34) |
| | WSFDPWGQGTLVTVSS | |
| | (SEQ ID NO: 33) | |
| ADI- | QVQLQQWGAGLLKPSETLSL | DIQMTQSPSTLSASVGDRVTITCRA |
| 29426 | TCAVYGGSFSGYYWSWIRQP | SQSIGSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYHSFPTFGG |
| | SSVTAADTAVYYCARARGP | GTKVEIK (SEQ ID NO: 36) |
| | WSFDPWGQGTLVTVSS | |
| | (SEQ ID NO: 35) | |
| ADI- | QVQLQQWGAGLLKPSETLSL | DIQMTQSPSTLSASVGDRVTITCRA |
| 29429 | TCAVYGG SFS GYYWSWIRQP | SQSIGSWLAWYQQKPGKAPKLLIY |
| | PGKGLEWIGEIDHSGSTNYNP | KASSLESGVPSRFSGSGSGTEFTLTI |
| | SLKSRVTISVDTSKNQFSLKL | SSLQPDDFATYYCQQYELYSYTFG |
| | SSVTAADTAVYYCARARGP | GGTKVEIK (SEQ ID NO: 38) |
| | WSFDPWGQGTLVTVSS | |
| | (SEQ ID NO: 37) | |
| ADI- | QVQLQQWGAGLLKPSETLSL | DIQMTQSPSTLSASVGDRVTITCRA |
| 29447 | TCAVYGGSFSGYYWSWIRQP | SQSISSWLAWYQQKPGKAPKLLIY |
| (F47) | PGKGLEWIGEIDHSGSTNYNP | KASSLESGVPSRFSGSGSGTEFTLTI |
| | SLKSRVTISVDTSKNQFSLKL | SSLQPDDFATYYCQQYDTFITFGG |
| | SSVTAADTAVYYCARARGP | GTKVEIK (SEQ ID NO: 40) |
| | WSFDPWGQGTLVTVSS | |
| | (SEQ ID NO: 39) | |
| ADI- | QVQLVQSGAEVKKPGSSVKV | DIVMTQSPDSLAVSLGERATINCKS |
| 27727 | SCKASGGTFSSYAISWVRQAP | SQSVLYSSNNKNYLAWYQQKPGQ |
| | GQGLEWMGGIIPIFGTANYAQ | PPKLLIYWASTRESGVPDRFSGSGS |
| | KFQGRVTITADESTSTAYMEL | GTDFTLTISSLQAEDVAVYYCQQY |
| | SSLRSEDTAVYYCARGDSSIR | YSTPITFGGGTKVEIK |
| | HAYYYYGMDVWGQGTTVT | (SEQ ID NO: 42) |
| | VSS (SEQ ID NO: 41) | CDR1: KSSQSVLYSSNNKNYLA |
| | CDR1: GTFSSYAIS (non-Kabat) | (SEQ ID NO: 46) |
| | (SEQ ID NO: 43) or SYAIS | CDR2: WASTRES (SEQ ID NO: 47) |
| | (SEQ ID NO:144) | CDR3: QQYYSTPIT (SEQ ID NO: |
| | CDR2: GIIPIFGTANYAQKFQG | 48) |
| | (SEQ ID NO: 44) | |
| | CDR3: | |
| | ARGDSSIRHAYYYYGMDV | |
| | (non-Kabat) (SEQ ID NO: 45) | |
| | or GDSSIRHAYYYYGMDV | |
| | (SEQ ID NO: 145) | |
| ADI- | QLQLQESGPGLVKPSETLSLT | EIVLTQSPATLSLSPGERATLSCRA |
| 29443 | CTVSGGSISSSSYYWGWIRQP | SQSVSRYLAWYQQKPGQAPRLLIY |
| (F43) | PGKGLEWIGSIYYSGSTYYNP | DASNRATGIPARFSGSGSGTDFTLT |
| | SLKSRVTISVDTSKNQFSLKL | ISSLEPEDFAVYYCQQFDTWPPTFG |
| | SSVTAADTAVYYCARGSDRF | GGTKVEIK (SEQ ID NO: 50) |
| | HPYFDYWGQGTLVTVSS | CDR1: RASQSVSRYLA |
| | (SEQ ID NO:49) | (SEQ ID NO: 54) |
| | CDR1: GSISSSSYYWG (non- | CDR2: DASNRAT (SEQ ID NO: 55) |
| | Kabat) (SEQ ID NO: 51) or | CDR3: QQFDTWPPT |
| | SSSYYWG (SEQ ID NO: 146) | (SEQ ID NO: 56) |
| | CDR2: SIYYSGSTYYNPSLKS | |
| | (SEQ ID NO: 52) | |
| | CDR3: ARGSDRFHPYFDY | |
| | (non-Kabat) (SEQ ID NO: 53) | |
| | or GSDRFHPYFDY | |
| | (SEQ ID NO: 147) | |
| ADI- | QVQLQQWGAGLLKPSETLSL | DIQMTQSPSTLSASVGDRVTITCRA |
| 29404 | TCAVYGGSFSGYYWSWIRQP | SQSISSWLAWYQQKPGKAPKLLIY |
| (F04) | PGKGLEWIGEIDHSGSTNYNP | KASSLESGVPSRFSGSGSGTEFTLTI |
| | SLKSRVTISVDTSKNQFSLKL | SSLQPDDFATYYCEQYDSYPTFGG |
| | SSVTAADTAVYYCARARGP | GTKVEIK (SEQ ID NO: 58) |
| | WSFDPWGQGTLVTVSS | |
| | (SEQ ID NO: 57) | |
| ADI- | QVQLVQSGAEVKKPGSSVKV | DIVMTQSPDSLAVSLGERATINCES |
| 28200 | SCKASGGTFSSYAISWVRQAP | SQSLLNSGNQKNYLTWYQQKPGQ |
| | GQGLEWMGGIIPIFGTANYAQ | PPKPLIYWASTRESGVPDRFSGSGS |
| | KFQGRVTITADESTSTAYMEL | GTDFTLTISSLQAEDVAVYYCQND |
| | SSLRSEDTAVYYCARRGRKA | YSYPYTFGQGTKLEIK |
| | SGSFYYYYGMDVWGQGTTV | (SEQ ID NO: 60) |
| | TVSS (SEQ ID NO: 59) | CDR1: ESSQSLLNSGNQKNYLT |
| | CDR1: GTFSSYAIS | (SEQ ID NO: 111) |
| | (SEQ ID NO: 108) | CDR2: WASTRES (SEQ ID NO: 112) |
| | CDR2: GIIPIFGTANYAQKFQG | CDR3: QNDYSYPYT |
| | (SEQ ID NO: 109) | (SEQ ID NO: 113) |
| | CDR3: | |
| | ARRGRKASGSFYYYYGMDV | |
| | (SEQ ID NO: 110) | |
| ADI- | QVQLVQSGAEVKKPGASVK | EIVMTQSPATLSVSPGERATLSCRA |
| 29379 | VSCKASGYTFTSYYMHWVR | SQSVSSNLAWYQQKPGQAPRLLIY |
| (E79) | QAPGQGLEWMGIINPSGGSTS | GASTRATGIPARFSGSGSGTEFTLTI |
| | YAQKFQGRVTMTRDTSTSTV | SSLQSEDFAVYYCQQYDDWPFTF |
| | YMELSSLRSEDTAVYYCARG | GGGTKVEIK (SEQ ID NO: 62) |
| | APNYGDTTHDYYYMDVWG | CDR1: RASQSVSSNLA |
| | KGTTVTVSS (SEQ ID NO: 61) | (SEQ ID NO: 66) |
| | CDR1: YTFTSYYMH (non- | CDR2: GASTRAT (SEQ ID NO: 67) |
| | Kabat) (SEQ ID NO: 63) or | CDR3: QQYDDWPFT |
| | SYYMH (SEQ ID NO: 148) | (SEQ ID NO: 68) |
| | CDR2: IINPSGGSTSYAQKFQG | |
| | (SEQ ID NO: 64) | |
| | CDR3: | |
| | ARGAPNYGDTTHDYYYMDV | |
| | (non-Kabat) (SEQ ID NO: 65) | |
| | or GAPNYGDTTHDYYYMDV | |
| | (SEQ ID NO: 149) | |
| ADI- | QVQLVQSGAEVKKPGASVK | EIVLTQSPGTLSLSPGERATLSCRA |
| 29463 | VSCKASGYTFTGYYMHWVR | SQSVSSNLAWYQQKPGQAPRLLIY |
| (F63) | QAPGQGLEWMGWINPNSGG | GASTRATGIPARFSGSGSGTEFTLTI |
| | TNYAQKFQGRVTMTRDTSIS | SSLQSEDFAVYYCQQDDYWPPTF |
| | TAYMELSRLRSDDTAVYYCA | GGGTKVEIK (SEQ ID NO: 70) |
| | RDTGEYYDTDDHGMDVWGQ | CDR1: RASQSVSSNLA |
| | GTTVTVSS (SEQ ID NO: 69) | (SEQ ID NO: 74) |
| | CDR1: YTFTGYYMH (non- | CDR2: GASTRAT (SEQ ID NO: 75) |
| | Kabat) (SEQ ID NO:71) or | CDR3: QQDDYWPPT |
| | GYYMH (SEQ ID NO: 150) | (SEQ ID NO: 76) |
| | CDR2: | |
| | WINPNSGGTNYAQKFQG | |
| | (SEQ ID NO: 72) | |
| | CDR3: | |
| | ARDTGEYYDTDDHGMDV | |
| | (non-Kabat) (SEQ ID NO: 73) | |
| | or DTGEYYDTDDHGMDV | |
| | (SEQ ID NO: 151) | |
| ADI- | EVQLLESGGGLVQPGGSLRLS | DIQMTQSPSSVSASVGDRVTITCRA |
| 27744 | CAASGFTFSSYAMSWVRQAP | SQGIDSWLAWYQQKPGKAPKLLI |
| (A44) | GKGLEWVSAISGSGGSTYYA | YAASSLQSGVPSRFSGSGSGTDFTL |
| | DSVKGRFTISRDNSKNTLYLQ | TISSLQPEDFATYYCQQGVSYPRTF |
| | MNSLRAEDTAVYYCAKDGG | GGGTKVEIK (SEQ ID NO: 78) |
| | YYDSGAGDYWGQGTLVTVS | CDR1: RASQGIDSWLA |
| | S (SEQ ID NO:77) | (SEQ ID NO: 82) |
| | CDR1: FTFSSYAMS (non- | CDR2: AASSLQS (SEQ ID NO: 83) |
| | Kabat) (SEQ ID NO:79) or | CDR3: QQGVSYPRT |
| | SYAMS (SEQ ID NO: 152) | (SEQ ID NO: 84) |
| | CDR2: | |
| | AISGSGGSTYYADSVKG | |
| | (SEQ ID NO: 80) | |
| | CDR3: AKDGGYYDSGAGDY | |
| | (non-Kabat) (SEQ ID NO: 81) | |
| | or DGGYYDSGAGDY | |
| | (SEQ ID NO: 153) | |
| ADI- | EVQLVESGGGLVKPGGSLRL | DIQMTQSPSSVSASVGDRVTITCRA |
| 27749 | SCAASGFTFSSYSMNWVRQA | SQGISSWLAWYQQKPGKAPKLLIY |
| (A49) | PGKGLEWVSSISSSSSYIYYA | AASSLQSGVPSRFSGSGSGTDFTLT |
| | DSVKGRFTISRDNAKNSLYLQ | ISSLQPEDFATYYCQQGVSFPRTFG |
| | MNSLRAEDTAVYYCARGAP | GGTKVEIK (SEQ ID NO: 86) |
| | MGAAAGWFDPWGQGTLVTV | CDR1: RASQGISSWLA |
| | SS (SEQ ID NO: 85) | (SEQ ID NO: 90) |
| | CDR1: FTFSSYSMN (non- | CDR2: AASSLQS (SEQ ID NO: 91) |
| | Kabat) (SEQ ID NO: 87) or | CDR3: QQGVSFPRT |
| | SYSMN (SEQ ID NO: 154) | (SEQ ID NO: 92) |
| | CDR2: SISSSSSYIYYADSVKG | |
| | (SEQ ID NO: 88) | |
| | CDR3: | |
| | ARGAPMGAAAGWFDP (non- | |
| | Kabat) (SEQ ID NO: 89) or | |
| | GAPMGAAAGWFDP | |
| | (SEQ ID NO: 155) | |
| ADI- | QVQLVQSGAEVKKPGASVK | EIVLTQSPATLSLSPGERATLSCRA |
| 29378 | VSCKASGYTFTSYYMHWVR | SQSVSSYLAWYQQKPGQAPRLLIY |
| (E78) | QAPGQGLEWMGIINPSGGSTS | DASNRATGIPARFSGSGSGTDFTLT |
| | YAQKFQGRVTMTRDTSTSTV | ISSLEPEDFAVYYCQQSDNWPFTF |
| | YMELSSLRSEDTAVYYCARE | GGGTKVEIK (SEQ ID NO: 94) |
| | GAGFAYGMDYYYMDVWGK | CDR1: RASQSVSSYLA |
| | GTTVTVSS (SEQ ID NO: 93) | (SEQ ID NO: 98) |
| | CDR1: YTFTSYYMH (non- | CDR2: DASNRAT (SEQ ID NO: 99) |
| | Kabat) (SEQ ID NO: 95) or | CDR3: QQSDNWPFT |
| | SYYMH (SEQ ID NO: 156) | (SEQ ID NO: 100) |
| | CDR2: IINPSGGSTSYAQKFQG | |
| | (SEQ ID NO: 96) | |
| | CDR3: | |
| | AREGAGFAYGMDYYYMDV | |
| | (non-Kabat) (SEQ ID NO: 97) | |
| | or EGAGFAYGMDYYYMDV | |
| | (SEQ ID NO: 157) | |
| A49MI | EVQLVESGGGLVKPGGSLRL | DIQMTQSPSSVSASVGDRVTITCRA |
| | SCAASGFTFSSYSMNWVRQA | SQGISSWLAWYQQKPGKAPKLLIY |
| | PGKGLEWVSSISSSSSYIYYA | AASSLQSGVPSRFSGSGSGTDFTLT |
| | DSVKGRFTISRDNAKNSLYLQ | ISSLQPEDFATYYCQQGVSFPRTFG |
| | MNSLRAEDTAVYYCARGAPI | GGTKVEIK (SEQ ID NO: 86) |
| | GAAAGWFDPWGQGTLVTVS | CDR1: RASQGISSWLA |
| | S (SEQ ID NO: 158) | (SEQ ID NO: 90) |
| | CDR1: FTFSSYSMN (non- | CDR2: AASSLQS (SEQ ID NO:91) |
| | Kabat) (SEQ ID NO: 87) or | CDR3: QQGVSFPRT |
| | SYSMN (SEQ ID NO: 159) | (SEQ ID NO: 92) |
| | CDR2: SISSSSSYIYYADSVKG | |
| | (SEQ ID NO: 88) | |
| | CDR3: ARGAPIGAAAGWFDP | |
| | (non-Kabat) (SEQ ID NO: 160) | |
| | or GAPIGAAAGWFDP | |
| | (SEQ ID NO: 161) | |
| A49MQ | EVQLVESGGGLVKPGGSLRL | DIQMTQSPSSVSASVGDRVTITCRA |
| | SCAASGFTFSSYSMNWVRQA | SQGISSWLAWYQQKPGKAPKLLIY |
| | PGKGLEWVSSISSSSSYIYYA | AASSLQSGVPSRFSGSGSGTDFTLT |
| | DSVKGRFTISRDNAKNSLYLQ | ISSLQPEDFATYYCQQGVSFPRTFG |
| | MNSLRAEDTAVYYCARGAP | GGTKVEIK (SEQ ID NO: 86) |
| | QGAAAGWFDPWGQGTLVTV | CDR1: RASQGISSWLA |
| | SS (SEQ ID NO: 162) | (SEQ ID NO: 90) |
| | CDR1: FTFSSYSMN (non- | CDR2: AASSLQS (SEQ ID NO: 91) |
| | Kabat) (SEQ ID NO: 87) or | CDR3: QQGVSFPRT |
| | SYSMN (SEQ ID NO: 163) | (SEQ ID NO: 92) |
| | CDR2: SISSSSSYIYYADSVKG | |
| | (SEQ ID NO: 88) | |
| | CDR3: ARGAPQGAAAGWFDP | |
| | (non-Kabat) (SEQ ID NO: 164) | |
| | or GAPQGAAAGWFDP | |
| | (SEQ ID NO: 165) | |
| A49ML | EVQLVESGGGLVKPGGSLRL | DIQMTQSPSSVSASVGDRVTITCRA |
| | SCAASGFTFSSYSMNWVRQA | SQGISSWLAWYQQKPGKAPKLLIY |
| | PGKGLEWVSSISSSSSYIYYA | AASSLQSGVPSRFSGSGSGTDFTLT |
| | DSVKGRFTISRDNAKNSLYLQ | ISSLQPEDFATYYCQQGVSFPRTFG |
| | MNSLRAEDTAVYYCARGAP | GGTKVEIK (SEQ ID NO: 86) |
| | LGAAAGWFDPWGQGTLVTV | CDR1: RASQGISSWLA |
| | SS (SEQ ID NO: 166) | (SEQ ID NO: 90) |
| | CDR1: FTFSSYSMN (non- | CDR2: AASSLQS (SEQ ID NO: 91) |
| | Kabat) (SEQ ID NO: 87) or | CDR3: QQGVSFPRT |
| | SYSMN (SEQ ID NO: 167) | (SEQ ID NO: 92) |
| | CDR2: SISSSSSYIYYADSVKG | |
| | (SEQ ID NO: 88) | |
| | CDR3: ARGAPLGAAAGWFDP | |
| | (non-Kabat) (SEQ ID NO: 168) | |
| | or GAPLGAAAGWFDP | |
| | (SEQ ID NO: 169) | |
| A49MF | EVQLVESGGGLVKPGGSLRL | DIQMTQSPSSVSASVGDRVTITCRA |
| | SCAASGFTFSSYSMNWVRQA | SQGISSWLAWYQQKPGKAPKLLIY |
| | PGKGLEWVSSISSSSSYIYYA | AASSLQSGVPSRFSGSGSGTDFTLT |
| | DSVKGRFTISRDNAKNSLYLQ | ISSLQPEDFATYYCQQGVSFPRTFG |
| | MNSLRAEDTAVYYCARGAPF | GGTKVEIK (SEQ ID NO: 86) |
| | GAAAGWFDPWGQGTLVTVS | CDR1: RASQGISSWLA |
| | S (SEQ ID NO: 170) | (SEQ ID NO:90) |
| | CDR1: FTFSSYSMN (non- | CDR2: AASSLQS (SEQ ID NO: 91) |
| | Kabat) (SEQ ID NO: 87) or | CDR3: QQGVSFPRT |
| | SYSMN (SEQ ID NO: 171) | (SEQ ID NO: 92) |
| | CDR2: SISSSSSYIYYADSVKG | |
| | (SEQ ID NO: 88) | |
| | CDR3: ARGAPFGAAAGWFDP | |
| | (non-Kabat) (SEQ ID NO: 172) | |
| | or GAPFGAAAGWFDP | |
| | (SEQ ID NO: 173) | |
| A49MV | EVQLVESGGGLVKPGGSLRL | DIQMTQSPSSVSASVGDRVTITCRA |
| | SCAASGFTFSSYSMNWVRQA | SQGISSWLAWYQQKPGKAPKLLIY |
| | PGKGLEWVSSISSSSSYIYYA | AASSLQSGVPSRFSGSGSGTDFTLT |
| | DSVKGRFTISRDNAKNSLYLQ | ISSLQPEDFATYYCQQGVSFPRTFG |
| | MNSLRAEDTAVYYCARGAP | GGTKVEIK (SEQ ID NO: 86) |
| | VGAAAGWFDPWGQGTLVTV | CDR1: RASQGISSWLA |
| | SS (SEQ ID NO:174) | (SEQ ID NO: 90) |
| | CDR1: FTFSSYSMN (non- | CDR2: AASSLQS (SEQ ID NO: 91) |
| | Kabat) (SEQ ID NO: 87) or | CDR3: QQGVSFPRT |
| | SYSMN (SEQ ID NO: 175) | (SEQ ID NO: 92) |
| | CDR2: SISSSSSYIYYADSVKG | |
| | (SEQ ID NO: 88) | |
| | CDR3: ARGAPVGAAAGWPDP | |
| | (non-Kabat) (SEQ ID NO: 176) | |
| | or GAPVGAAAGWFDP | |
| | (SEQ ID NO: 177) | |
| A49- | EVQLVESGGGLVKPGGSLRL | DIQMTQSPSSVSASVGDRVTITCRA |
| consensus | SCAASGFTFSSYSMNWVRQA | SQGISSWLAWYQQKPGKAPKLLIY |
| | PGKGLEWVSSISSSSSYIYYA | AASSLQSGVPSRFSGSGSGTDFTLT |
| | DSVKGRFTISRDNAKNSLYLQ | ISSLQPEDFATYYCQQGVSFPRTFG |
| | MNSLRAEDTAVYYCARGAP | GGTKVEIK (SEQ ID NO: 86) |
| | XGAAAGWFDPWGQGTLVTV | CDR1: RASQGISSWLA |
| | SS, wherein X is M, L, I, V, | (SEQ ID NO: 90) |
| | Q, or F (SEQ ID NO: 178) | CDR2: AASSLQS (SEQ ID NO: 91) |
| | CDR1: FTFSSYSMN (non- | CDR3: QQGVSFPRT |
| | Kabat) (SEQ ID NO: 87) or | (SEQ ID NO: 92) |
| | SYSMN (SEQ ID NO: 179) | |
| | CDR2: SISSSSSYIYYADSVKG | |
| | (SEQ ID NO: 88) | |
| | CDR3: ARGAPXGAAAGWPDP | |
| | (non-Kabat) (SEQ ID NO: 180) | |
| | or GAPXGAAAGWFDP, wherein | |
| | X is M, L, I, V, Q, or F | |
| | (SEQ ID NO: 181) | |

Alternatively, a heavy chain variable domain represented by SEQ ID NO:101 can be paired with a light chain variable domain represented by SEQ ID NO:102 to form an antigen-binding site that can bind to NKG2D, as illustrated in U.S. Pat. No. 9,273,136.

SEQ ID NO: 101
QVQLVESGGGLVKPGGSLRLSCAASGFTFSSYGMHWVRQAPGKGLEWVAF
IRYDGSNKYYADSVKGRFTISRDNSKNTLYLQMNSLRAEDTAVYYCAKDR
GLGDGTYFDYWGQGTTVTVSS
SEQ ID NO: 102
QSALTQPASVSGSPGQSITISCSGSSSNIGNNAVNWYQQLPGKAPKLLIY
YDDLLPSGVSDRFSGSKSGTSAFLAISGLQSEDEADYYCAAWDDSLNGPV
FGGGTKLTVL

Alternatively, a heavy chain variable domain represented by SEQ ID NO:103 can be paired with a light chain variable domain represented by SEQ ID NO:104 to form an antigen-binding site that can bind to NKG2D, as illustrated in U.S. Pat. No. 7,879,985.

SEQ ID NO: 103
QVHLQESGPGLVKPSETLSLTCTVSDDSISSYYWSWIRQPPGKGLEWIGH
ISYSGSANYNPSLKSRVTISVDTSKNQFSLKLSSVTAADTAVYYCANWDD
AFNIWGQGTMVTVSS
SEQ ID NO: 104
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK

In one aspect, the present disclosure provides multi-specific binding proteins that bind to the NKG2D receptor and CD16 receptor on natural killer cells, and the antigen P-cadherin (CDH3). Table 2 lists some exemplary sequences of heavy chain variable domains and light chain variable domains that, in combination, can bind to P-cadherin (CDH3). CDR sequences of the heavy and light chain variable domain amino acid sequences listed in Table 2 below and described in the corresponding patents and publications are incorporated by reference herein. Unless indicated otherwise, the CDR sequences provided in Table 2 are determined under Kabat.

TABLE 2

| | Heavy | |
|---|---|---|
| | chain variable domain | Light chain variable domain |
| Source | amino acid sequence | amino acid sequence |
| PF-03732010 | EVQLLESGGGLVQPGGSLR | QSALTQPASVSGSPGQSITISCTG |
| US | LSCAASGFTFSSYAMSWVR | TSNDVGAYNYVSWYQQHPGKA |
| 20110182884 | QAPGKGLEWVSAISGSGGS | PKLMISEVNKRPSGVSNRFSGSK |
| (U.S. Pat. No. | TYYADSVKGRFTISRDNSK | SGNTASLTISGLQAEDEADYYCS |
| 8,974,781) | NTLYLQMNSLRAEDTAVY | SFTSGLPWVVFGGGTKLTVL |
| | YCAKWGDGTLNPWGQGT | (SEQ ID NO: 118) |
| | MVTVSS (SEQ ID NO: 114) | CDR1: SNDVGAYNYVS |
| | CDR1: GFTFSSY | (SEQ ID NO: 119) or |
| | (SEQ ID NO: 115) | TGTSNDVGAYNYVS |
| | CDR2: SGSGGS | (SEQ ID NO: 225) |
| | (SEQ ID NO: 116) | CDR2: EVNKRPS |
| | CDR3: WGDGTLNP | (SEQ ID NO: 120) |
| | (SEQ ID NO: 117) | CDR3: SSFTSGLPWVV |
| | | (SEQ ID NO: 121) |
| U.S. Pat. | EVQLVESGGGLVQPGGSLR | DIQMTQSPSSLSASVGDRVTITC |
| No. | LSCAASGFSLTSYGVHWVR | RASQNIYSNLAWYQQKPGKAP |
| 9,644,028 | QAPGKGLEWVGVIWSGGST | KLLVYAAKNLQSGVPSRFSGSG |
| | DYADSVKGRFTISRDNSKN | SGTDFTLTISSLQPEDFATYYCQ |
| | TLYLQMNSLRAEDTAVYYC | HFYDTPWTFGQGTKVEIK |
| | ARNSNNGFAYWGQGTLVT | (SEQ ID NO: 126) |
| | VSS (SEQ ID NO: 122) | CDR1: NIYSNLA |
| | CDR1: SLTSYGVH | (SEQ ID NO: 127) |
| | (SEQ ID NO: 123) | CDR2: LLVYAAKN |
| | CDR2: GVIWSGGSTD | (SEQ ID NO: 128) |
| | (SEQ ID NO: 124) | CDR3: QHFYDTPWT |
| | CDR3: ARNSNNGFAY | (SEQ ID NO: 129) |
| | (SEQ ID NO: 125) | |
| NOV | QVQLQQSGPGLVKPSQTLS | DIQMTQSPSSLSASVGDRVTITC |
| 169N31Q | LTCAISGDSVSSQSAAWNW | RASQTISNTLAWYQQKPGKAPK |
| US Patent | IRQSPSRGLEWLGRIYYRSK | LLIYAASNLQSGVPSRFSGSGSG |
| Publication | WYNDYALSVKSRITINPDTS | TDFTLTISSLQPEDFATYYCQQY |
| NO. | KNQFSLQLNSVTPEDTAVY | LSWFTFGQGTKVEIK |
| 20160137730 | YCARGEGYGREGFAIWGQ | (SEQ ID NO: 134) |
| A1 | GTLVTVSS (SEQ ID NO: 130) | CDR1: RASQTISNTLA |
| (U.S. Pat. | CDR1: SQSAAWN | (SEQ ID NO: 135) or SQTISNT |
| No.: | (SEQ ID NO: 131) or | (non-Kabat) (SEQ ID NO: 185) |
| 10,005,836) | GDSVSSQSA (non-Kabat) | CDR2: AASNLQS |
| | (SEQ ID NO: 182) | (SEQ ID NO: 136) or AAS (non- |
| | CDR2: | Kabat) (SEQ ID NO: 186) |
| | RIYYRSKWYNDYALSVKS | CDR3: QQYLSWFT |
| | (SEQ ID NO: 132) or | (SEQ ID NO: 137) or YLSWF |
| | YYRSKWY (non-Kabat) | (non-Kabat) (SEQ ID NO: 187) |
| | (SEQ ID NO: 183) | |
| | CDR3: GEGYGREGFAI | |
| | (SEQ ID NO: 133) or | |
| | GEGYGREGFAI (non-Kabat) | |
| | (SEQ ID NO: 184) | |
| TSP7 | QVQLQESGPELVKPGASVK | DIDIQMTQTTSSLSASLGDRVTIS |
| | MSCKASGYSFTAYNMHWV | CRASQDITNYLNWYQQKPDGT |
| | KQSHGKSLEWIGFIDPYSGII | VKLLIYYTSRLHSGVPSRFSGSG |
| | TYNQTFKGKATLTVDKSSS | SGTDYSLTISNLEQEDIATYFCQ |
| | TAYMQLNSLTSEDSAVYYC | QDSKHPRTFGGGTKLEIK |
| | ARRGYYDGGFDYWGQGTT | (SEQ ID NO: 192) |
| | LTVSSS (SEQ ID NO: 188) | CDR1: RASQDITNYLN |
| | CDR1: GYSFTAYNMH | (SEQ ID NO: 193) |
| | (SEQ ID NO: 189) | CDR2: YTSRLHS |
| | CDR2: IDPYSGIIT | (SEQ ID NO: 194) |
| | (SEQ ID NO: 190) | CDR3: QQDSKHPRT |
| | CDR3: RGYYDGGFDY | (SEQ ID NO: 195) |
| | (SEQ ID NO: 191) | |
| PF-06671008 | EVQLVQSGAEVKKPGASVK | QSVLTQPPSVSAAPGQKVTISCS |
| or | VSCKASGYTFTSYGISWVR | GSSSNIGNNYVSWYQQLPGTAP |
| DART153 | QAPGQGLEWMGWISAYNG | KLLIYDNNKRPSGIPDRFSGSKS |
| (D153) | NTNYAQKLQGRVTMTTDT | GTSATLGITGLQTGDEADYYCG |
| WO | STSTAYMELRSLRSDDTAV | TWDSSLSGVVFGGGTKLTVL |
| 2016001810 | YYCATIDTANAFGIWGQGT | (SEQ ID NO: 200) |
| | MVTVSS (SEQ ID NO: 196) | CDR1: SGSSSNIGNNYVS |
| | CDR1: GYTFTSYGIS (non- | (SEQ ID NO: 201) |
| | Kabat) (SEQ ID NO: 197) | CDR2: DNNKRPS |
| | CDR2: WISAYNGNTN (non- | (SEQ ID NO: 202) |
| | Kabat) (SEQ ID NO: 198) | CDR3: GTWDSSLSGVV |
| | CDR3: IDTANAFGI | (SEQ ID NO: 203) |
| | (SEQ ID NO: 199) | |
| DART20 | EVQLVQSGAEVKKPGASVK | AIQMTQSPSSLSASVGDRVTITC |
| WO | VSCKASGYTFTGYYMHWV | RASQSIGKYLNWYQQIPGKAPK |
| 2016001810 | RQAPGQGLEWMGRINPNSG | LLIYTASTLQTGVPSRFSGSGSG |
| | GTNYAQKFQGRVTMTRDT | TDFTLTIVSLQPEDFATYYCQQS |
| | SISTAYMELSRLRSDDTAVY | FNTPRTFGQGTKVEIK |
| | YCAKGSGSGAFDIWGQGT | (SEQ ID NO: 208) |
| | MVTVSS (SEQ ID NO: 204) | CDR1: RASQSIGKYLN |
| | CDR1: GYTFTGYYMH | (SEQ ID NO: 209) |
| | (SEQ ID NO: 205) | CDR2: TASTLQT |
| | CDR2: RINPNSGGTN | (SEQ ID NO: 210) |
| | (SEQ ID NO: 206) | CDR3: QQSFNTPRT |
| | CDR3: GSGSGAFDI | (SEQ ID NO: 211) |
| | (SEQ ID NO: 207) | |
| DART30 | EVQLVESGGGVVQPGRSLR | DIVMTQSPLSLPVTLGQPASISC |
| WO | LSCAASGFTFSSYGMHWVR | RSSQSLVHSDGNTYLHWFQQRP |
| 2016001810 | QAPGKGLEWVAVISYDGSN | GQSPRRLIYKVSNRDSGVPDRFS |
| | KYYADSVKGRFTISRDNSK | GSGSGTDFTLKISRVEAEDVGV |
| | NTLYLQMNSLRAEDTAVY | YYCMQGTHWPPLFGQGTKVEI |
| | YCAKSSSDRTFDYWGQGTL | K (SEQ ID NO: 216) |
| | VTVSS (SEQ ID NO: 212) | CDR1: RSSQSLVHSDGNTYLH |
| | CDR1: GFTFSSYGMH | (SEQ ID NO: 217) |
| | (SEQ ID NO: 213) | CDR2: KVSNRDS |
| | CDR2: VISYDGSNKY | (SEQ ID NO: 218) |
| | (SEQ ID NO: 214) | CDR3: MQGTHWPPL |
| | CDR3: SSSDRTFDY | (SEQ ID NO: 219) |
| | (SEQ ID NO: 215) | |
| 196-H02 | EVQLLESGGGLVQPGGSLR | QSALTQPASVSGSPGQSITISCTG |
| | LSCAASGFTFSSYAMSWVR | TSNDVGAYNYVSWYQQHPGKA |
| | QAPGKGLEWVSAISGSGGS | PKLILSEVNKRPSGVSNRFSGSK |
| | TYYADSVKGRFTISRDNSK | SGNTASLSISGLQAEDEADYYCT |
| | NTLYLQMNSLRAEDTAVY | SYTMGSTFMLFGGGTKLTVL |
| | YCADTNSAKFDPWGQGTM | (SEQ ID NO: 224) |
| | VTVSS (SEQ ID NO: 220) | CDR1: TGTSNDVGAYNYVS |
| | CDR1: GFTFSSYAMS | (SEQ ID NO: 225) |
| | (SEQ ID NO :221) | CDR2: EVNKRPS |
| | CDR2: AISGSGGSTY | (SEQ ID NO: 120) |
| | (SEQ ID NO: 222) | CDR3: TSYTMGSTFML |
| | CDR3: TNSAKFDP | (SEQ ID NO: 226) |
| | (SEQ ID NO: 223) | |
| 198-A09 | EVQLLESGGGLVQPGGSLR | QSALTQPASVSGPPGQSITISCTG |
| | LSCAASGFTFSSYAMSWVR | TSNDVGAYNYVSWYQQHPGKA |
| | QAPGKGLEWVSAISGSGGS | PKLILSEVNKRPSGVSNRFSGSK |
| | TYYADSVKGRFTISRDNSK | SGNTASLSISGLQAEDEADYYCT |
| | NTLYLQMNSLRAEDTAVY | SYTMGSTFMLFGGGTKLTVL |
| | YCATIAPGRFDPWGQGTMV | (SEQ ID NO: 229) |
| | TVSS (SEQ ID NO: 227) | CDR1: TGTSNDVGAYNYVS |
| | CDR1: GFTFSSYAMS | (SEQ ID NO: 225) |
| | (SEQ ID NO: 221) | CDR2: EVNKRPS |
| | CDR2: AISGSGGSTY | (SEQ ID NO: 120) |
| | (SEQ ID NO: 222) | CDR3: TSYTMGSTFML |
| | CDR3: IAPGRFDP | (SEQ ID NO: 226) |
| | (SEQ ID NO: 228) | |

Alternatively, novel antigen-binding sites that can bind to P-cadherin can be identified by screening for binding to the amino acid sequence defined by SEQ ID NO:138 (CDH3 isoform 1 precursor), SEQ ID NO:139 (CDH3 isoform 2 precursor), or SEQ ID NO:140 (CDH3 isoform 3 precursor).

SEQ ID NO: 138
MGLPRGPLASLLLLQVCWLQCAASEPCRAVIREAEVTLEAGGAEQEPGQA
LGKVFMGCPGQEPALFSTDNDDFTVRNGETVQERRSLKERNPLKIFPSKR
ILRRHKRDWVVAPISVPENGKGPFPQRLNQLKSNKDRDTKIFYSITGPGA
DSPPEGVFAVEKETGWLLLNKPLDREEIAKYELFGHAVSENGASVEDPMN
ISIIVTDQNDHKPKFTQDTFRGSVLEGVLPGTSVMQVTATDEDDAIYTYN
GVVAYSIHSQEPKDPHDLMFTIHRSTGTISVISSGLDREKVPEYTLTIQA
TDMDGDGSTTTAVAVVEILDANDNAPMFDPQKYEAHVPENAVGHEVQRLT
VTDLDAPNSPAWRATYLIMGGDDGDHFTITTHPESNQGILTTRKGLDFEA
KNQHTLYVEVTNEAPFVLKLPTSTATIVVHVEDVNEAPVFVPPSKVVEVQ
EGIPTGEPVCVYTAEDPDKENQKISYRILRDPAGWLAMDPDSGQVTAVGT
LDREDEQFVRNNIYEVMVLAMDNGSPPTTGTGTLLLTLIDVNDHGPVPEP
RQITICNQSPVRQVLNITDKDLSPHTSPFQAQLTDDSDIYWTAEVNEEGD
TVVLSLKKFLKQDTYDVHLSLSDHGNKEQLTVIRATVCDCHGHVETCPGP
WKGGFILPVLGAVLALLFLLLVLLLLVRKKRKIKEPLLLPEDDTRDNVFY
YGEEGGGEEDQDYDITQLHRGLEARPEVVLRNDVAPTIIPTPMYRPRPAN
PDEIGNFIIENLKAANTDPTAPPYDTLLVFDYEGSGSDAASLSSLTSSAS
DQDQDYDYLNEWGSRFKKLADMYGGGEDD
SEQ ID NO: 139
MGLPRGPLASLLLLQVCWLQCAASEPCRAVIREAEVTLEAGGAEQEPGQA
LGKVFMGCPGQEPALFSTDNDDFTVRNGETVQERRSLKERNPLKIFPSKR
ILRRHKRDWVVAPISVPENGKGPFPQRLNQLKSNKDRDTKIFYSITGPGA
DSPPEGVFAVEKETGWLLLNKPLDREEIAKYELFGHAVSENGASVEDPMN
ISIIVTDQNDHKPKFTQDTFRGSVLEGVLPGTSVMQVTATDEDDAIYTYN
GVVAYSIHSQEPKDPHDLMFTIHRSTGTISVISSGLDREKVPEYTLTIQA
TDMDGDGSTTTAVAVVEILDANDNAPMFDPQKYEAHVPENAVGHEVQRLT
VTDLDAPNSPAWRATYLIMGGDDGDHFTITTHPESNQGILTTRKGLDFEA
KNQHTLYVEVTNEAPFVLKLPTSTATIVVHVEDVNEAPVFVPPSKVVEVQ
EGIPTGEPVCVYTAEDPDKENQKISYRILRDPAGWLAMDPDSGQVTAVGT
LDREDEQFVRNNIYEVMVLAMDNGSPPTTGTGTLLLTLIDVNDHGPVPEP
RQITICNQSPVRQVLNITDKDLSPHTSPFQAQLTDDSDIYWTAEVNEEGD
TVVLSLKKFLKQDTYDVHLSLSDHGNKEQLTVIRATVCDCHGHVETCPGP
WKGGFILPVLGAVLALLFLLLVLLLLVRKKRKIKEPLLLPEDDTRDNVFY
YGEEGGGEEDQDYDITQLHRGLEARPEVVLRNDVAPTIIPTPMYRPRPAN
PDEIGNFIIEGRGERGSQRGNGGLQLARGRTRRS
SEQ ID NO: 140
MGCPGQEPALFSTDNDDFTVRNGETVQERRSLKERNPLKIFPSKRILRRH
KRDWVVAPISVPENGKGPFPQRLNQLKSNKDRDTKIFYSITGPGADSPPE
GVFAVEKETGWLLLNKPLDREEIAKYELFGHAVSENGASVEDPMNISIIV
TDQNDHKPKFTQDTFRGSVLEGVLPGTSVMQVTATDEDDAIYTYNGVVAY
SIHSQEPKDPHDLMFTIHRSTGTISVISSGLDREKVPEYTLTIQATDMDG
DGSTTTAVAVVEILDANDNAPMFDPQKYEAHVPENAVGHEVQRLTVTDLD
APNSPAWRATYLIMGGDDGDHFTITTHPESNQGILTTRKGLDFEAKNQHT
LYVEVTNEAPFVLKLPTSTATIVVHVEDVNEAPVFVPPSKVVEVQEGIPT
GEPVCVYTAEDPDKENQKISYRILRDPAGWLAMDPDSGQVTAVGTLDRED
EQFVRNNIYEVMVLAMDNGSPPTTGTGTLLLTLIDVNDHGPVPEPRQITI
CNQSPVRQVLNITDKDLSPHTSPFQAQLTDDSDIYWTAEVNEEGDTVVLS
LKKFLKQDTYDVHLSLSDHGNKEQLTVIRATVCDCHGHVETCPGPWKGGF
ILPVLGAVLALLFLLLVLLLLVRKKRKIKEPLLLPEDDTRDNVFYYGEEG
GGEEDQDYDITQLHRGLEARPEVVLRNDVAPTIIPTPMYRPRPANPDEIG
NFIIENLKAANTDPTAPPYDTLLVFDYEGSGSDAASLSSLTSSASDQDQD
YDYLNEWGSRFKKLADMYGGGEDD

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, e.g., Sondermann P., et al. (2000) Nature; 406(6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cx of a human IgG1 constant region may be at amino acid E123, F116, S176, V163, S174, and/or T164.

Alternatively, amino acid substitutions could be selected from the following sets of substitutions shown in Table 3.

TABLE 3

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 4.

TABLE 4

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 5.

TABLE 5

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 6.

TABLE 6

| First Polypeptide | Second Polypeptide |
|---|---|
| L351Y, D399R, D399K, S400K, | T366V, T366I, T366L, T366M, |
| S400R, Y407A, Y407I, Y407V | N390D, N390E, K392L, K392M, |
| | K392V, K392F K392D, K392E, |
| | K409F, K409W, T411D and T411E |

Alternatively, at least one amino acid substitution could be selected from the following set of substitutions in Table 7, where the position(s) indicated in the First Polypeptide column is replaced by any known negatively-charged amino acid, and the position(s) indicated in the Second Polypeptide Column is replaced by any known positively-charged amino acid.

TABLE 7

| First Polypeptide | Second Polypeptide |
|---|---|
| K392, K370, K409, or K439 | D399, E356, or E357 |

Alternatively, at least one amino acid substitution could be selected from the following set of substitutions in Table 8, where the position(s) indicated in the First Polypeptide column is replaced by any known positively-charged amino acid, and the position(s) indicated in the Second Polypeptide Column is replaced by any known negatively-charged amino acid.

TABLE 8

| First Polypeptide | Second Polypeptide |
|---|---|
| D399, E356, or E357 | K409, K439, K370, or K392 |

Alternatively, amino acid substitutions could be selected from the following set in Table 9.

TABLE 9

| First Polypeptide | Second Polypeptide |
|---|---|
| T350V, L351Y, F405A, and | T350V, T366L, K392L, and |
| Y407V | T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, S400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, S400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

The multi-specific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, flow cytometry, microscopy, or Clonepix.

Clones can be cultured under conditions suitable for bio-reactor scale-up and maintained expression of the multi-specific protein. The multispecific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Proteins

The multi-specific binding proteins described herein include an NKG2D-binding site, a CD16-binding site, and a binding site for P-cadherin. In some embodiments, the multi-specific binding proteins bind to cells expressing NKG2D and/or CD16, such as NK cells, and tumor cells expressing P-cadherin simultaneously. Binding of the multi-specific proteins to NK cells can enhance the activity of the NK cells toward destruction of the cancer cells.

In certain embodiments, the multi-specific binding proteins described herein bind to P-cadherin with a similar affinity to that of a corresponding monoclonal antibody having the same P-cadherin binding site. In certain embodiments, the multi-specific binding proteins described herein are more effective at killing tumor cells expressing P-cadherin than a corresponding monoclonal antibody having the same P-cadherin binding site.

In certain embodiments, the multi-specific binding proteins described herein, which include an NKG2D-binding site and a binding site for P-cadherin, activate primary human NK cells when co-cultured with tumor cells expressing P-cadherin. NK cell activation is marked by the increase in CD107a expression, degranulation and IFN-γ cytokine production. Furthermore, compared to a corresponding monoclonal antibody having the same P-cadherin binding site, the multi-specific binding proteins described herein may show superior activation of human NK cells in the presence of cells expressing P-cadherin.

In certain embodiments, the multi-specific binding proteins described herein, which include an NKG2D-binding site and a binding site for P-cadherin, enhance the activation of resting and IL-2-activated human NK cells in the presence of tumor cells expressing P-cadherin.

In certain embodiments, compared to a corresponding monoclonal antibody having the same P-cadherin binding site, the multi-specific binding proteins described herein can have greater cytotoxic activity against tumor cells having medium or low expression of P-cadherin.

### III. Therapeutic Applications

The invention provides methods for treating cancer using a multi-specific binding protein described herein and/or a pharmaceutical composition described herein. The methods may be used to treat a variety of cancers expressing P-cadherin. Exemplary cancers to be treated may be gastric cancer, colorectal cancer, pancreatic cancer, breast cancer, endometrial cancer, lung cancer, prostate cancer, bladder cancer, cervical cancer, head and neck cancer, ovarian cancer, esophageal cancer, renal cancer, liver cancer, testicular cancer, oral cavity cancer, multiple myeloma, leukemia, acute myeloid leukemia, melanoma, basocellular and squamous cell carcinomas of the skin, glioma, Ewing sarcoma, Kaposi's sarcoma, and mesothelioma.

In some other embodiments, exemplary cancers to be treated may be acral lentiginous melanoma, actinic keratoses, acute lymphoblastic leukemia, acute lymphocytic leukemia, acute myeloid leukemia, adenocarcinoma, adenoid cystic carcinoma, adenosarcoma, adenosquamous carcinoma, anal canal cancer, anaplastic large cell lymphoma, angioimmunoblastic T-cell lymphoma, angiosarcoma, anorectal cancer, astrocytic tumor, bartholin gland carcinoma, basocellular carcinomas (e.g., skin), B-cell lymphoma, biliary tract cancer, bladder cancer, bone cancer, bone marrow cancer, brain cancer, breast cancer, bronchial cancer, bronchial gland carcinoma, Burkitt lymphoma, carcinoid, cervical cancer, cholangiocarcinoma, chondrosarcoma, choroid plexus papilloma/carcinoma, chronic lymphocytic leukemia, chronic myeloid leukemia, chronic neutrophilic leukemia, clear cell carcinoma, colon cancer, colorectal cancer, connective tissue cancer, cutaneous T-cell lymphoma, cystadenoma, diffuse large B-cell lymphoma, digestive system cancer, duodenum cancer, endocrine system cancer, endodermal sinus tumor, endometrial cancer/hyperplasia, endometrial stromal sarcoma, endometrioid adenocarcinoma, endothelial cell cancer, enteropathy type T-cell lymphoma, ependymal cancer, epithelial cell cancer, esophageal cancer, Ewing sarcoma, extranodal marginal zone B-cell lymphoma, extranodal natural killer/T-cell lymphoma, eye and orbit cancer, female genital cancer, focal nodular hyperplasia, follicular lymphoma, gall bladder cancer, gastric antrum cancer, gastric cancer, gastric fundus cancer, gastrinoma, glioblastoma, glioma, glucagonoma, hairy cell leukemia, head and neck cancer, heart cancer, hemangioblastoma, hemangioendothelioma, hemangiomas, hematological tumors, hepatic adenoma, hepatic adenomatosis, hepatocellular carcinoma, hepatobilliary cancer, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, kidney cancer, large cell carcinoma, large intestine cancer, leiomyosarcoma, lentigo maligna melanomas, leukemia, liver cancer, lung cancer, lymphoma, lymphoplasmacytic lymphoma, male genital cancer, malignant melanoma, malignant mesothelial tumors, mantle cell lymphoma, marginal zone B-cell lymphoma, medulloblastoma, medulloepithelioma, melanoma, meningeal cancer, mesothelial cancer, mesothelioma, metastatic carcinoma, mouth cancer, mucoepidermoid carcinoma, multiple myeloma, muscle cancer, myelodysplastic neoplasms, myeloproliferative neoplasms, nasal tract cancer, nervous system cancer, neuroblastoma, neuroepithelial adenocarcinoma, nodal marginal zone B-cell lymphoma, nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, parotid cancer, pelvic cancer, penile cancer, peripheral T-cell lymphoma, pharynx cancer, pituitary tumors, plasmacytoma, precursor T-lymphoblastic lymphoma, primary central nervous system lymphoma, primary mediastinal B-cell lymphoma, prostate cancer, pseudosarcoma, pulmonary blastoma, rectal cancer, renal cancer, renal cell carcinoma, respiratory system cancer, retinoblastoma, rhabdomyosarcoma, sarcoma, serous carcinoma, sinus cancer, skin cancer, small cell carcinoma, small intestine cancer, small lymphocytic lymphoma, smooth muscle cancer, soft tissue cancer, somatostatin-secreting tumor, spine cancer, splenic marginal zone B-cell lymphoma, squamous cell carcinoma (e.g., skin), striated muscle cancer, subcutaneous panniculitis-like T-cell lymphoma, submesothelial cancer, superficial spreading melanoma, T cell leukemia, T cell lymphoma, testicular cancer, thyroid cancer, tongue cancer, undifferentiated carcinoma, ureter cancer, urethra cancer, urinary bladder cancer, uterine cancer, uterine corpus cancer, uveal melanoma, vaginal cancer, verrucous carcinoma, VIPoma, vulva cancer, well-differentiated carcinoma, or Wilms tumor.

### IV. Combination Therapy

Another aspect of the invention provides for combination therapy. A multi-specific binding protein described herein can be used in combination with additional therapeutic agents to treat a cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK inhibitor, an ATR inhibitor, an A2A antagonist, a base excision repair inhibitor, a Bcr-Abl tyrosine kinase inhibitor, a Bruton's tyrosine kinase inhibitor, a CDCl₇ inhibitor, a CHK1 inhibitor, a Cyclin-Dependent kinase inhibitor, a DNA-PK inhibitor, an inhibitor of both DNA-PK and mTOR, a DNMT1 inhibitor, a DNMT1 inhibitor plus 2-chloro-deoxyadenosine, an HDAC inhibitor, a Hedgehog signaling pathway inhibitor, an IDO inhibitor, a JAK inhibitor, an mTOR Inhibitor, a MEK inhibitor, a MELK inhibitor, a MTH1 inhibitor, a PARP inhibitor, a phosphoinositide 3-kinase inhibitor, an inhibitor of both PARP1 and DHODH, a proteasome inhibitor, a topoisomerase-II Inhibitor, a tyrosine kinase inhibitor, a VEGFR inhibitor, and a WEE inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, 17th Ed. Mack Publishing Company, Easton, Pa. (1985). For a brief review of methods for drug delivery, see, e.g., Langer T. (1990) Science; 249(4976):1527-1533.

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtain a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

This present disclosure could exist in a liquid aqueous pharmaceutical formulation including a therapeutically effective amount of the multi-specific protein in a buffered solution.

The compositions disclosed herein may be sterilized by conventional sterilization techniques, or may be filter-sterilized. The resulting aqueous solutions may be packaged for use as-is, or lyophilized, wherein the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the multi-specific protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (e.g., sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/mL of disodium phosphate dihydrate, 0.7 to 1.1 mg/mL of sodium dihydrogen phosphate dihydrate, and 6.0 to 6.4 mg/mL of sodium chloride. In certain embodiments, the pH of the formulation is adjusted with sodium hydroxide.

A polyol, which acts as a tonicifier and may stabilize an antibody, may also be included in the formulations described herein. The polyol is added to a formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to 15 mg/mL. In certain embodiments, the concentration of mannitol may be about 10-14 mg/mL. In certain embodiments, the concentration of mannitol may be about 12 mg/mL. In certain embodiments, the polyol sorbitol may be included in the formulation.

A detergent or surfactant may also be added to the formulations of the present invention. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see, e.g., Fiedler H. P., Lexikon der Hifsstoffe für Pharmazie, Kosmetik und andrenzende Gebiete, 4^{th} Ed., Editio Cantor, Aulendorf, Germany (1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the multi-specific protein product of the present disclosure is formulated as a liquid formulation. The liquid formulation may be present at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the multi-specific protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments, the liquid formulation may be prepared in an aqueous carrier. In certain embodiments, a stabilizer may be added in an amount no greater than that which may result in a viscosity undesirable or unsuitable for intravenous administration. In certain embodiments, the sugar may be a disaccharide, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variation of peptides and proteins that may occur during fermentation, harvest/cell clarification, purification, drug substance/drug product storage, and sample analysis. Under physiological conditions, deamidation is the loss of ammonia (NH₃) from an asparagine residue of a protein, resulting in a 17 dalton decrease in mass and formation of a succinimide intermediate. Subsequent hydrolysis of succinimide results in an 18 dalton mass increase and formation of aspartic acid or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain may also affect deamidation rates, e.g., Gly and Ser residues following an Asn residue results in a higher susceptibility to deamidation.

In certain embodiments, the liquid formulation of the present disclosure may be preserved under conditions of pH and humidity to prevent deamidation of the protein product.

The aqueous carrier of interest herein is one which is pharmaceutically acceptable (i.e., safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% sodium chloride solution before administration. In certain embodiments, the diluted drug product for injection is isotonic and suitable for administration by intravenous infusion.

In certain embodiments, salt or buffer components may be added in amounts of about 10 mM to about 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, or citrate buffers, in which case, sodium, potassium or ammonium ions can serve as counterions.

A preservative may be optionally added to the formulations herein to reduce bacterial action. The addition of a preservative may, for example, facilitate the production of a multi-use (i.e., multiple-dose) formulation.

The aqueous carrier of interest herein is one which is pharmaceutically acceptable (i.e., safe and non-toxic for administration to a human) and is useful for the preparation of a liquid formulation. Illustrative carriers include SWFI, BWFI, a pH buffered solution (e.g., phosphate-buffered saline), sterile saline solution, Ringer's solution or dextrose solution.

This present disclosure could exist in a lyophilized formulation including the proteins and a lyoprotectant. The lyoprotectant may be a sugar, e.g., a disaccharide. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be from 1:2 to 1:5.

In certain embodiments, the pH of the lyophilized formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments of the lyophilized formulation, salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added to the lyophilized formulation. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the lyophilized formulations herein to reduce bacterial action. The addition of a preservative may, for example, facilitate the production of a multi-use (i.e., multiple-dose) formulation.

In certain embodiments, the lyophilized drug product may be reconstituted with an aqueous diluent. The aqueous diluent of interest herein is one which is pharmaceutically acceptable (e.g., safe and non-toxic for administration to a human) and is useful for the preparation of a reconstituted liquid formulation, after lyophilization. Illustrative diluents include SWFI, BWFI, a pH buffered solution (e.g., phosphate-buffered saline), sterile saline solution, Ringer's solution or dextrose solution.

In certain embodiments, the lyophilized drug product of the current disclosure is reconstituted with either SWFI, USP or 0.9% sodium chloride for injection, USP. During reconstitution, the lyophilized powder dissolves into a solution.

In certain embodiments, the lyophilized protein product of the instant disclosure is constituted to about 4.5 mL in SWFI and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (see, e.g. Schmitz et al. (2001) Clinica Chimica Acta; 308: 43-53, 2001; Steimer et al. (2001) Clinica Chimica Acta 308: 33-41).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, or about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention can be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, or once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1—NKG2D Binding Domains Bind to NKG2D

### NKG2D Binding Domains Bind to Purified Recombinant NKG2D

The nucleic acid sequences of human, mouse or cynomolgus NKG2D ectodomains were fused with nucleic acid sequences encoding human IgG1 Fc domains and introduced into mammalian cells to be expressed. After purification, NKG2D-Fc fusion proteins were adsorbed to wells of microplates. After blocking the wells with bovine serum albumin to prevent non-specific binding, NKG2D-binding domains were titrated and added to the wells pre-adsorbed with NKG2D-Fc fusion proteins. Primary antibody binding was detected using a secondary antibody which was conjugated to horseradish peroxidase and specifically recognizes a human kappa light chain to avoid Fc cross-reactivity. 3,3′,5,5′-Tetramethylbenzidine (TMB), a substrate for horseradish peroxidase, was added to the wells to visualize the binding signal, whose absorbance was measured at 450 nM and corrected at 540 nM. An NKG2D-binding domain clone, an isotype control or a positive control (comprising heavy chain and light chain variable domains selected from SEQ ID NOs:101-104, or anti-mouse NKG2D clones MI-6 and CX-5, eBioscience, San Diego, Calif.) was added to each well.

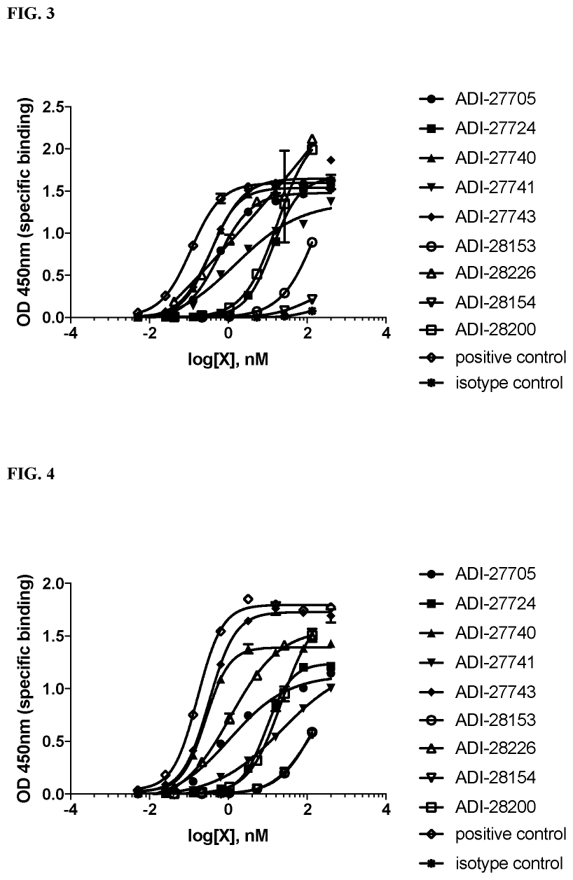

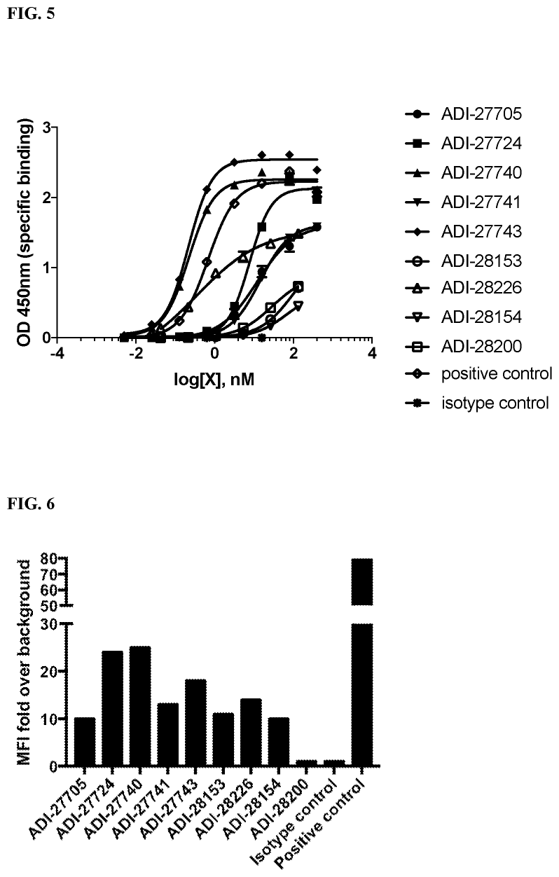

The isotype control showed minimal binding to recombinant NKG2D-Fc proteins, while the positive control bound strongest to the recombinant antigens. NKG2D-binding domains produced by all clones demonstrated binding across human, mouse, and cynomolgus recombinant NKG2D-Fc proteins, although with varying affinities from clone to clone. Generally, each anti-NKG2D clone bound to human (FIG. 3) and cynomolgus (FIG. 4) recombinant NKG2D-Fc with similar affinity, but with lower affinity to mouse (FIG. 5) recombinant NKG2D-Fc.

### NKG2D-Binding Domains Bind to Cells Expressing NKG2D

EL4 mouse lymphoma cell lines were engineered to express human or mouse NKG2D-CD3 zeta signaling domain chimeric antigen receptors. An NKG2D-binding clone, an isotype control or a positive control was used at a 100 nM concentration to stain extracellular NKG2D expressed on the EL4 cells. The antibody binding was detected using fluorophore-conjugated anti-human IgG secondary antibodies. Cells were analyzed by flow cytometry, and fold-over-background (FOB) was calculated using the mean fluorescence intensity (MFI) of NKG2D expressing cells compared to parental EL4 cells.

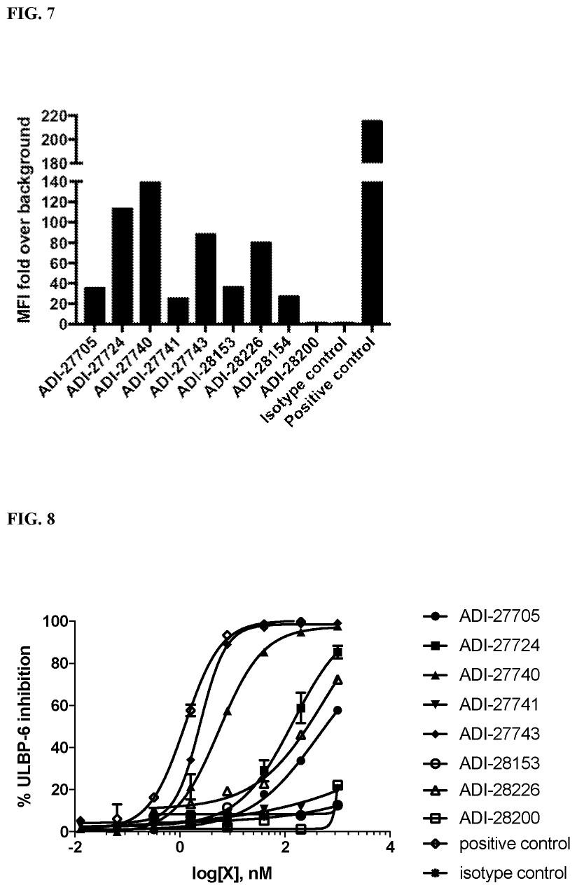

NKG2D-binding domains produced by all clones bound to EL4 cells expressing human and mouse NKG2D. Positive control antibodies (comprising heavy chain and light chain variable domains selected from SEQ ID NOs:101-104, or anti-mouse NKG2D clones MI-6 and CX-5 eBioscience, San Diego, Calif.) gave the best FOB binding signal. The NKG2D-binding affinity for each clone was similar between cells expressing human NKG2D (FIG. 6) and mouse (FIG. 7) NKG2D.

### Example 2—NKG2D-Binding Domains Block Natural Ligand Binding to NKG2D

Competition with ULBP-6

Recombinant human NKG2D-Fc proteins were adsorbed to wells of a microplate, and the wells were blocked with bovine serum albumin reduce non-specific binding. A saturating concentration of ULBP-6-His-biotin was added to the wells, followed by addition of the NKG2D-binding domain clones. After a 2-hour incubation, wells were washed and ULBP-6-His-biotin that remained bound to the NKG2D-Fc coated wells was detected by streptavidin-conjugated to horseradish peroxidase and TMB substrate. Absorbance was measured at 450 nM and corrected at 540 nM. After subtracting background, specific binding of NKG2D-binding domains to the NKG2D-Fc proteins was calculated from the percentage of ULBP-6-His-biotin that was blocked from binding to the NKG2D-Fc proteins in wells. The positive control antibody (comprising heavy chain and light chain variable domains selected from SEQ ID NOs:101-104) and various NKG2D-binding domains blocked ULBP-6 binding to NKG2D, while isotype control showed little competition with ULBP-6 (FIG. 8).

ULBP-6 sequence is represented by SEQ ID NO:141

(SEQ ID NO: 141)
MAAAAIPALLLCLPLLFLLFGWSRARRDDPHSLCYDITVIPKFRPGPRWC
AVQGQVDEKTFLHYDCGNKTVTPVSPLGKKLNVTMAWKAQNPVLREVVDI
LTEQLLDIQLENYTPKEPLTLQARMSCEQKAEGHSSGSWQFSIDGQTFLL
FDSEKRMWTTVHPGARKMKEKWENDKDVAMSFHYISMGDCIGWLEDFLMG
MDSTLEPSAGAPLAMSSGTTQLRATATTLILCCLLIILPCFILPGI

Competition with MICA

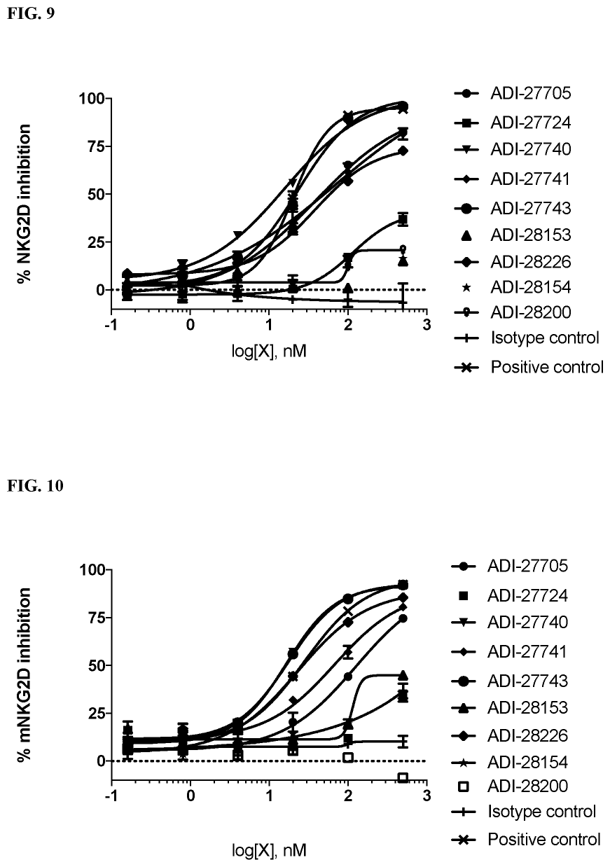

Recombinant human MICA-Fc proteins were adsorbed to wells of a microplate, and the wells were blocked with bovine serum albumin to reduce non-specific binding. NKG2D-Fc-biotin was added to wells followed by NKG2D-binding domains. After incubation and washing, NKG2D-Fc-biotin that remained bound to MICA-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the MICA-Fc coated wells. The positive control antibody (comprising heavy chain and light chain variable domains selected from SEQ ID NOs:101-104) and various NKG2D-binding domains blocked MICA binding to NKG2D, while isotype control showed little competition with MICA (FIG. 9).

Competition with Rae-1 Delta

Recombinant mouse Rae-1 delta-Fc (R&D Systems; Minneapolis, Minn.) was adsorbed to wells of a microplate, and the wells were blocked with bovine serum albumin to reduce non-specific binding. Mouse NKG2D-Fc-biotin was added to the wells followed by NKG2D-binding domains. After incubation and washing, NKG2D-Fc-biotin that remained bound to Rae-1delta-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the Rae-1delta-Fc coated wells. The positive control (comprising heavy chain and light chain variable domains selected from SEQ ID NOs:101-104, or anti-mouse NKG2D clones MI-6 and CX-5, eBioscience, San Diego, Calif.) and various NKG2D-binding domain clones blocked Rae-1delta binding to mouse NKG2D, while the isotype control antibody showed little competition with Rae-1delta (FIG. 10).

### Example 3—NKG2D-Binding Domain Clones Activate NKG2D

Nucleic acid sequences of human and mouse NKG2D were fused to nucleic acid sequences encoding a CD3 zeta signaling domain to obtain chimeric antigen receptor (CAR) constructs. The NKG2D-CAR constructs were then cloned into a retrovirus vector using Gibson assembly and transfected into expi293 cells for retrovirus production. EL4 cells were infected with viruses containing NKG2D-CAR together with 8 μg/mL polybrene. 24 hours after infection, the expression levels of NKG2D-CAR in the EL4 cells were analyzed by flow cytometry, and clones which express high levels of the NKG2D-CAR on the cell surface were selected.

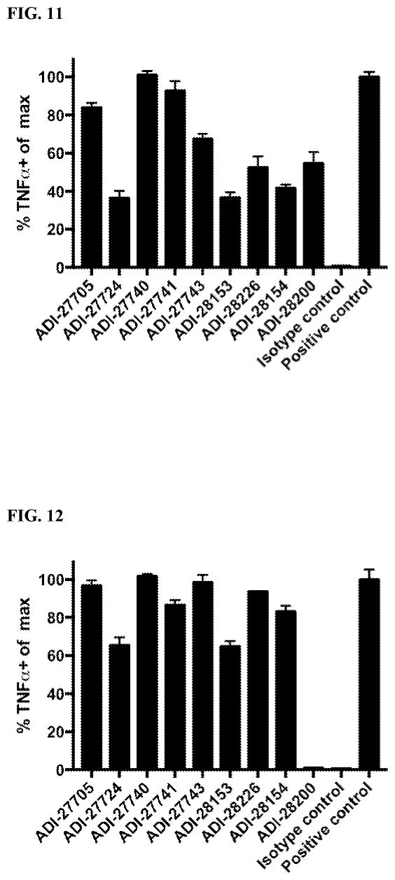

To determine whether NKG2D-binding domains activate NKG2D, they were adsorbed to wells of a microplate, and NKG2D-CAR EL4 cells were cultured on the antibody fragment-coated wells for 4 hours in the presence of brefeldin-A and monensin. Intracellular TNF-α production, an indicator for NKG2D activation, was assayed by flow cytometry. The percentage of TNF-α positive cells was normalized to the cells treated with the positive control. All NKG2D-binding domains activated both human NKG2D (FIG. 11) and mouse NKG2D (FIG. 12).

### Example 4—NKG2D-Binding Domains Activate NK Cells

### Primary Human NK Cells

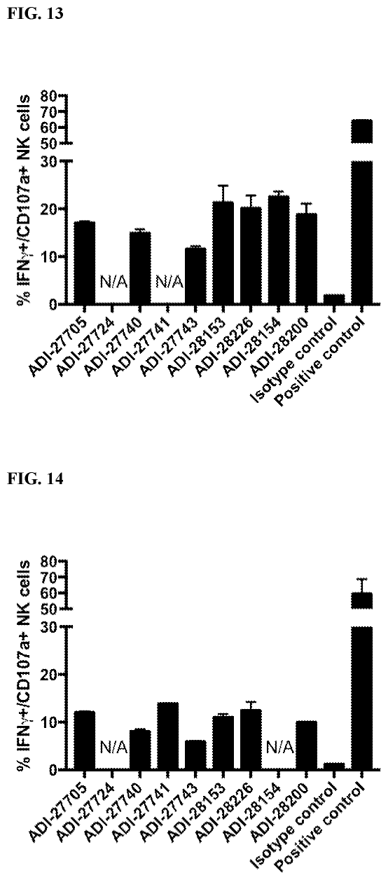

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which the NKG2D-binding domains were adsorbed, and cultured in the media containing fluorophore-conjugated anti-CD107a antibody, brefeldin-A, and monensin. Following culture, NK cells were assayed by flow cytometry using fluorophore-conjugated antibodies against CD3, CD56 and IFNγ. CD107a and IFN-γ staining were analyzed in CD3-CD56⁺ cells to assess NK cell activation. The increase in CD107a/IFNγ double-positive cells is indicative of better NK cell activation through engagement of two activating receptors rather than one receptor. NKG2D-binding domains and the positive control (e.g., heavy chain variable domain represent by SEQ ID NO:101 or SEQ ID NO: 103, and light chain variable domain represented by SEQ ID NO: 102 or SEQ ID NO: 104) showed a higher percentage of NK cells becoming CD107a⁺ and IFNγ⁺ than the isotype control (FIG. 13 and FIG. 14 represent data from two independent experiments, each using a different donor's PBMCs for NK cell preparation).

### Primary Mouse NK Cells

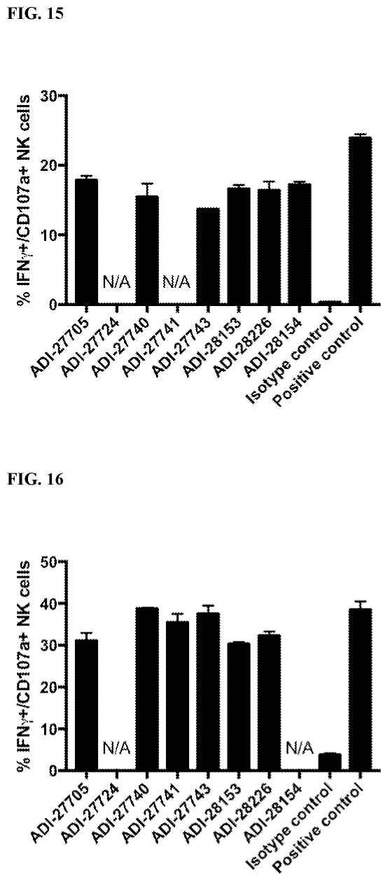

Spleens were obtained from C57Bl/6 mice and crushed through a 70 μm cell strainer to obtain a single cell suspension. Cells were pelleted and resuspended in ACK lysis buffer (Thermo Fisher Scientific #A1049201, Carlsbad, Calif.; 155 mM ammonium chloride, 10 mM potassium bicarbonate, 0.01 mM EDTA) to remove red blood cells. The remaining cells were cultured with 100 ng/mL hIL-2 for 72 hours before being harvested and prepared for NK cell isolation. NK cells (CD3-NK1.1⁺) were then isolated from spleen cells using a negative depletion technique with magnetic beads which typically yields NK cell populations having >90% purity. Purified NK cells were cultured in media containing 100 ng/mL mIL-15 for 48 hours before they were transferred to the wells of a microplate to which the NKG2D-binding domains were adsorbed, and cultured in media containing fluorophore-conjugated anti-CD107a antibody, brefeldin-A, and monensin. Following culture in NKG2D-binding domain-coated wells, NK cells were assayed by flow cytometry using fluorophore-conjugated antibodies against CD3, NK1.1 and IFN-γ. CD107a and IFN-γ staining were analyzed in CD3-NK1.1⁺ cells to assess NK cell activation. The increase in CD107a/IFN-γ double-positive cells is indicative of better NK cell activation through engagement of two activating receptors rather than one receptor. NKG2D-binding domains and the positive control (selected from anti-mouse NKG2D clones MI-6 and CX-5, eBioscience, San Diego, Calif.) showed a higher percentage of NK cells becoming CD107a⁺ and IFN-γ+ than the isotype control (FIG. 15 and FIG. 16 represent data from two independent experiments, each using a different mouse for NK cell preparation).

### Example 5—NKG2D-Binding Domains Enhance Cytotoxicity Against Target Tumor Cells

Human and mouse primary NK cell activation assays demonstrate increased cytotoxicity markers on NK cells after incubation with NKG2D-binding domains. To address whether this translates into increased tumor cell lysis, a cell-based assay was utilized where each NKG2D-binding domain was developed into a monospecific antibody. The Fc region was used as one targeting arm, while the Fab region (NKG2D-binding domain) acted as another targeting arm to activate NK cells. THP-1 cells, which are of human origin and express high levels of Fc receptors, were used as a tumor target and a Perkin Elmer DELFIA® Cytotoxicity Kit (Waltham, Mass.) was used. THP-1 cells were labeled with BATDA reagent, and resuspended at 10⁵ cells/mL in culture media. Labeled THP-1 cells were then combined with NKG2D antibodies and isolated mouse NK cells in wells of a microtiter plate at 37° C. for 3 hours. After incubation, 20 μl of the culture supernatant was removed, mixed with 200 μl of Europium solution and incubated with shaking for 15 minutes in the dark. Fluorescence was measured over time by a PHERAStar® plate reader equipped with a time-resolved fluorescence module (Excitation 337 nm, Emission 620 nm) and specific lysis was calculated according to the kit instructions.

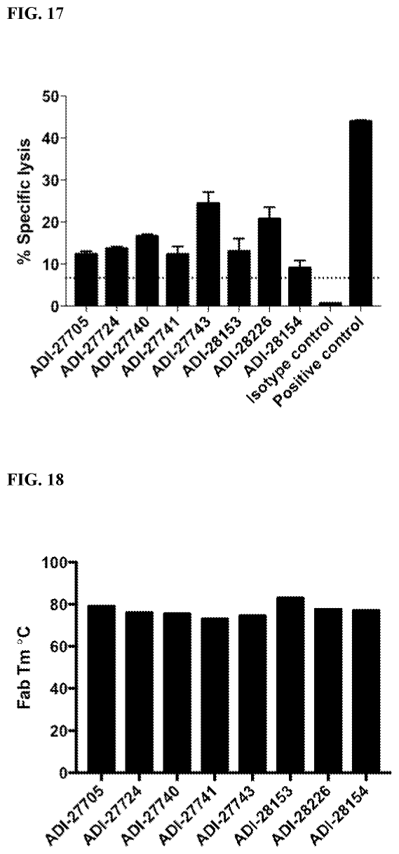

The positive control, ULBP-6—a natural ligand for NKG2D, showed increased specific lysis of THP-1 target cells by mouse NK cells. NKG2D antibodies also increased specific lysis of THP-1 target cells, while isotype control antibody showed reduced specific lysis. The dotted line indicates specific lysis of THP-1 cells by mouse NK cells without antibody added (FIG. 17).

### Example 6—NKG2D Antibodies have High Thermostability

Melting temperatures of NKG2D-binding domains were assayed using differential scanning fluorimetry. The extrapolated apparent melting temperatures of NKG2D-binding domains were high relative to typical IgG1 antibodies (FIG. 18).

### Example 7—Synergistic Activation of Human NK Cells by Cross-Linking NKG2D and CD16

### Primary Human NK Cell Activation Assay

Peripheral blood mononuclear cells (PBMCs) were isolated from peripheral human blood buffy coats using density gradient centrifugation. NK cells were purified from PBMCs using negative selection magnetic beads (StemCell Technologies, Vancouver, Canada; Cat #17955). NK cells were >90% CD3⁻CD56⁺ as determined by flow cytometry. Cells were then expanded 48 hours in media containing 100 ng/mL hIL-2 (PeproTech, Inc., Rocky Hill, N.J.; Cat #200-02) before use in activation assays. Antibodies were coated onto a 96-well flat-bottom plate at a concentration of 2 μg/ml (anti-CD16, BioLegend, San Diego, Calif.; Cat #302013) and 5 μg/mL (anti-NKG2D, R&D Systems, Minneapolis, Minn.; Cat #MAB139) in 100 μl sterile phosphate buffered saline (PBS) overnight at 4° C. followed by washing the wells thoroughly to remove excess antibody. For the assessment of degranulation, IL-2-activated NK cells were resuspended at 5×10⁵ cells/ml in culture media supplemented with 100 ng/mL hIL2 and 1 μg/mL APC-conjugated anti-CD107a mAb (BioLegend; Cat #328619). 1×10⁵ cells/well were then added onto antibody coated plates. The protein transport inhibitors Brefeldin A (BFA, BioLegend. San Diego, Calif.; Cat #420601) and Monensin (BioLegend, San Diego, Calif.; Cat #420701) were added at a final dilution of 1:1000 and 1:270 respectively. Plated cells were incubated for 4 hours at 37° C. in 5% CO₂. For intracellular staining of IFN-γ NK cells were labeled with anti-CD3 (BioLegend, San Diego, Calif.; Cat #300452) and anti-CD56 mAb (BioLegend, San Diego, Calif.; Cat #318328) and subsequently fixed and permeabilized and labeled with anti-IFN-γ mAb (BioLegend, San Diego Calif.; Cat #506507). NK cells were analyzed for expression of CD107a and IFN-γ by flow cytometry after gating on live CD56⁺CD3-cells.

To investigate the relative potency of receptor combination, crosslinking of NKG2D or CD16 and co-crosslinking of both receptors by plate-bound stimulation was performed.

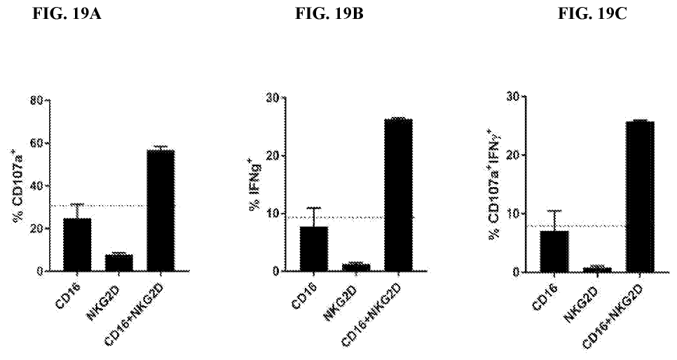

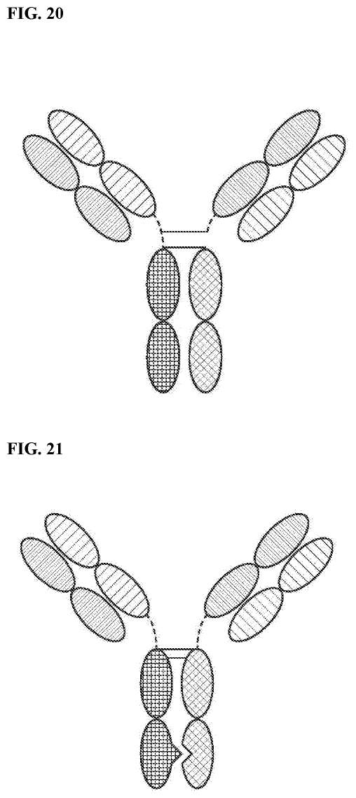

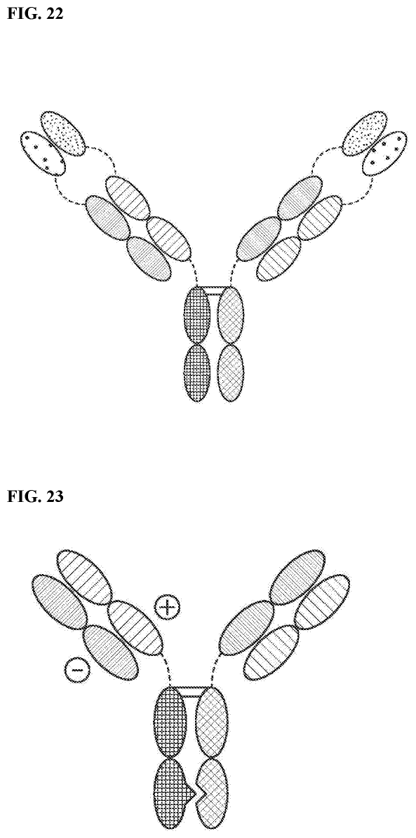

As shown in FIG. 19, expression of CD107a and intracellular IFNγ of IL-2-activated NK cells was analyzed after 4 hours of plate-bound stimulation with anti-CD16, anti-NKG2D, or a combination of both monoclonal antibodies. Combined stimulation of CD16 and NKG2D resulted in percentages of CD107a⁺ cells (FIG. 19A) and IFNγ⁺ cells (FIG. 19B) that were greater than the additive effect of individual stimulations of CD16 or NKG2D alone (as indicated by the dotted line). Similarly, combined stimulation of CD16 and NKG2D resulted in a greater percentage of CD107a⁺IFNγ⁺ double-positive cells as compared to the additive effect of individual of each receptor alone (FIG. 19C). Bar graphs show the mean (n=2) ±SD and are representative of five independent experiments using five different healthy donors.

### Example 8—Assessment of Multi-Specific Binding Protein and Monoclonal Antibody Binding to Cells Expressing P-Cadherin (CDH3)

Human P-cadherin (CDH3)-expressing cancer cell lines, HCT116, H1975 and CAL27, were used to assess tumor antigen binding of multi-specific binding proteins having a P-cadherin (CDH3) binding site comprising a heavy chain variable domain sequence identical to SEQ ID NO:196 and a light chain variable domain sequence identical to SEQ ID NO:200 (D153 DB), or a P-cadherin (CDH3) binding site comprising a heavy chain variable domain sequence identical to SEQ ID NO:188 and a light chain variable domain sequence identical to SEQ ID NO:192 (TSP7 DB). Multi-specific binding proteins or corresponding monoclonal antibodies (mAb) having the same P-cadherin (CDH3) binding site were diluted and incubated with the cells. Binding was detected using a fluorophore conjugated anti-human IgG secondary antibody. Cells were analyzed by flow cytometry and binding was expressed as mean fluorescence intensity (MFI) normalized to human recombinant IgG1 stained controls to obtain fold over background (FOB) values.

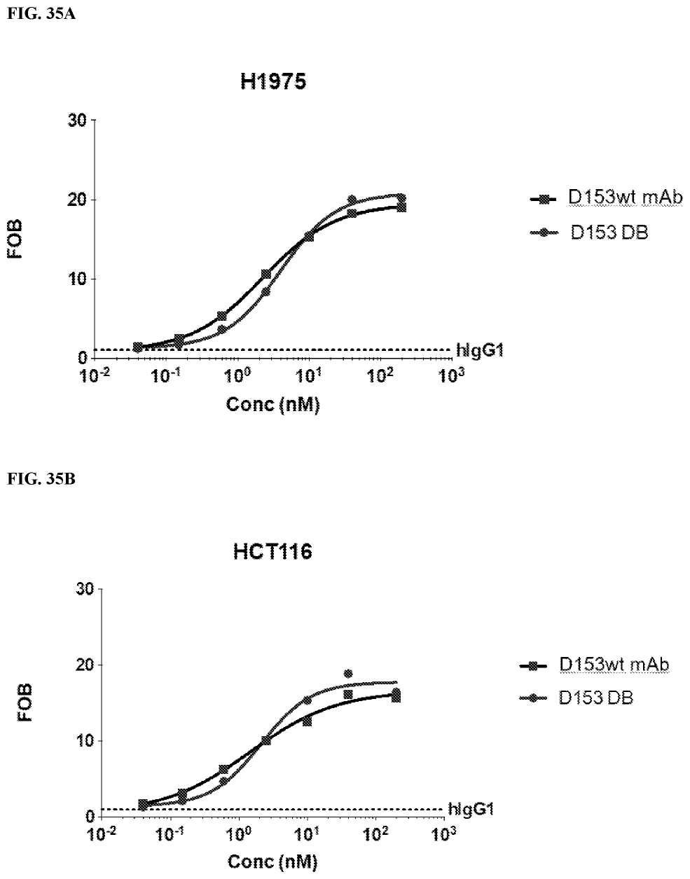

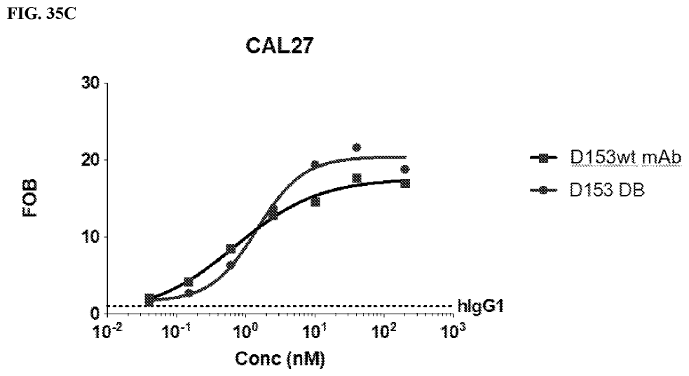

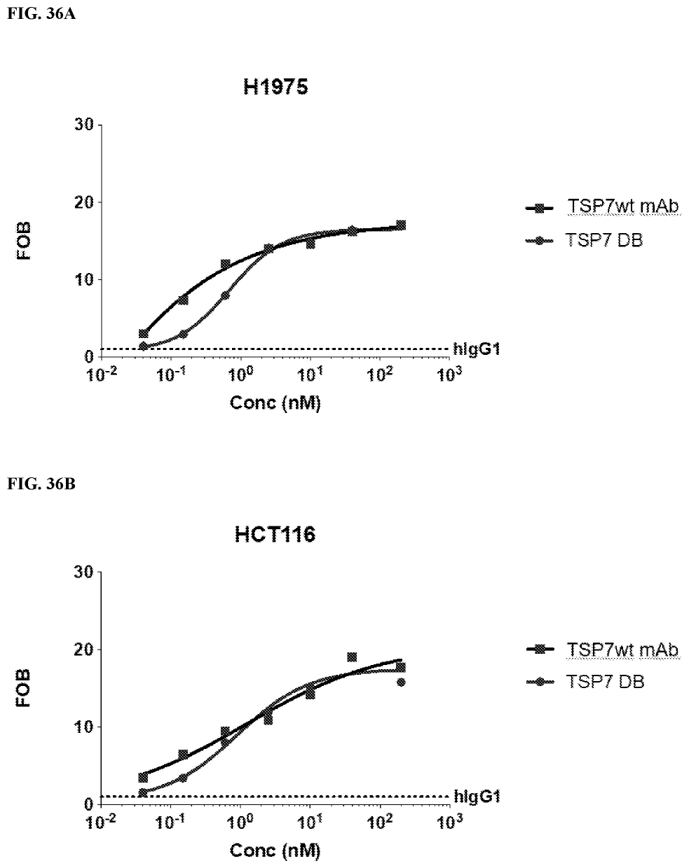

As shown in FIGS. 35A-C, anti-P-cadherin mAb (D153 wt mAb) including a heavy chain variable domain sequence identical to SEQ ID NO:196 and a light chain variable domain sequence identical to SEQ ID NO:200, and anti-P-cadherin multi-specific binding protein (D153 DB) having the same P-cadherin binding Fab, bind with a similar dose-response to P-cadherin expressing human non-small cell lung cancer cells (H1975; FIG. 35A), human colorectal cancer cells (HCT116; FIG. 35B), and human tongue squamous carcinoma cells (CAL27; FIG. 35C). Overall binding signal was higher with multi-specific binding proteins as compared to corresponding mAb.

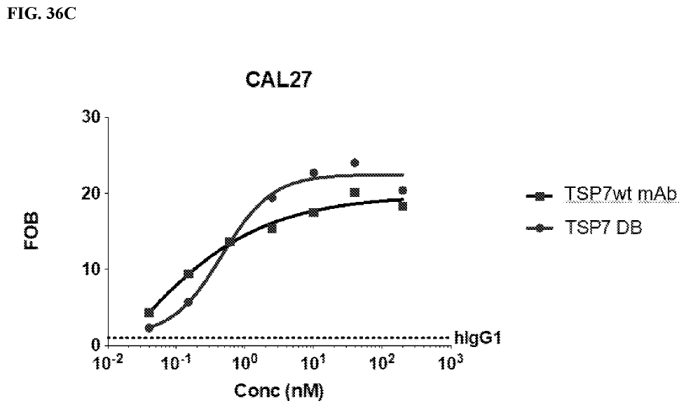

Similarly, as shown in FIGS. 36A-C, anti-P-cadherin mAb (TSP7 wt mAb) including a heavy chain variable domain sequence identical to SEQ ID NO:188 and a light chain variable domain sequence identical to SEQ ID NO:192, and anti-P-cadherin multi-specific binding protein (TSP7 DB) having the same P-cadherin binding Fab, bind with a similar dose response to P-cadherin-expressing H1975 (FIG. 36A), HCT116 (FIG. 36B), and CAL27 (FIG. 36C) cells.

### Example 9—Enhanced NK Cell-Mediated Lysis of P-Cadherin (CDH3)+Target Cells by Multi-Specific Binding Proteins

KHYG1-CD16V cells were produced by ectopically expressing the high affinity V158 allele of CD16 in KHYG1 cells. Cells were maintained in RPMI1650 media supplemented with 10% heat-inactivated fetal bovine serum, 1×GutaMAX™ (Life Technologies, Carlsbad, Calif.), 100 U/ml penicillin streptomycin, 50 μM P-mercaptoethanol and 100 ng/ml IL2. Cells were incubated overnight in IL2-free media before use in cytotoxicity assays.

PBMCs were isolated from human peripheral blood buffy coats using density gradient centrifugation. Isolated PBMCs were washed and prepared for NK cell isolation. NK cells were isolated using a negative selection with magnetic beads. NK cells were >90% CD3-CD56⁺ as determined by flow cytometry. Isolated NK cells were incubated overnight in cytokine-free media before use in cytotoxicity assays.

### DELFIA Cytotoxicity Assay:

P-cadherin-expressing human cancer cell lines were harvested from culture. Cells were washed with PBS, and resuspended in growth media at 10⁶ cells/mL for labeling with BATDA reagent (Perkin Elmer, Waltham, Mass., Cat #AD0116) in accordance with the manufacturer's instructions. After labeling, cells were washed 3× with PBS and resuspended at 5×10⁴ cells/mL in culture media and 100 μl of BATDA labeled cells were added to each well of a 96-well plate. Designated wells were reserved for spontaneous release from target cells, and all other wells were prepared for maximum lysis of target cells by addition of 1% Triton-X.

50 μl of diluted P-cadherein mAb or anti-P-cadherein duobody multi-specific binding protein was added to designated wells. KHYG1-CD16V or purified primary NK cells were harvested from culture, washed and resuspended at a concentration or 1×10⁵-2.0×10⁶ cells/mL in culture media. 50 μl of KHYG1-CD16V or primary NK cell suspension were added to designated wells of the 96-well plate to make a total of 200 μl culture volume and to achieve an effector to target cell ratio of 10:1. The plate was incubated at 37° C., 5% CO₂ for 2-4 hours before developing the assay.

Following co-culture, cells were pelleted by centrifugation at 200×G for 5 minutes. 20 μl of culture supernatant was transferred to a clean microplate and 200 μl of room temperature europium solution was added to each well. The microplate was protected from the light and incubated on a plate shaker at 250 rpm for 15 minutes. The microplate was read with a SpectraMax i3X instrument (Molecular Devices, San Jose, Calif.). % Specific lysis was calculated as follows:

% Specific lysis=[(Experimental release−Spontaneous release)/(Maximum release−Spontaneous release)]×100%

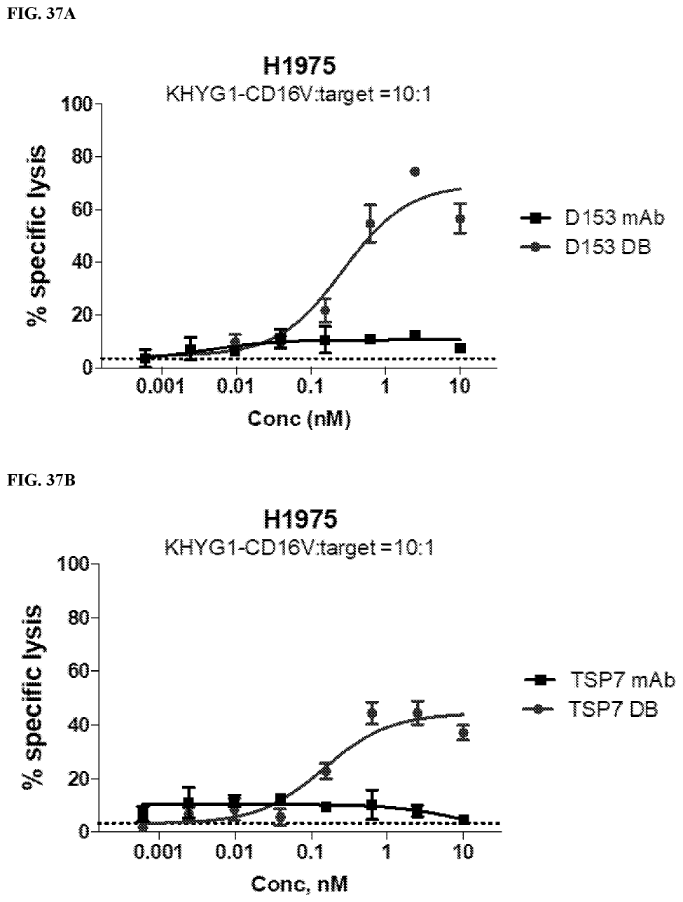

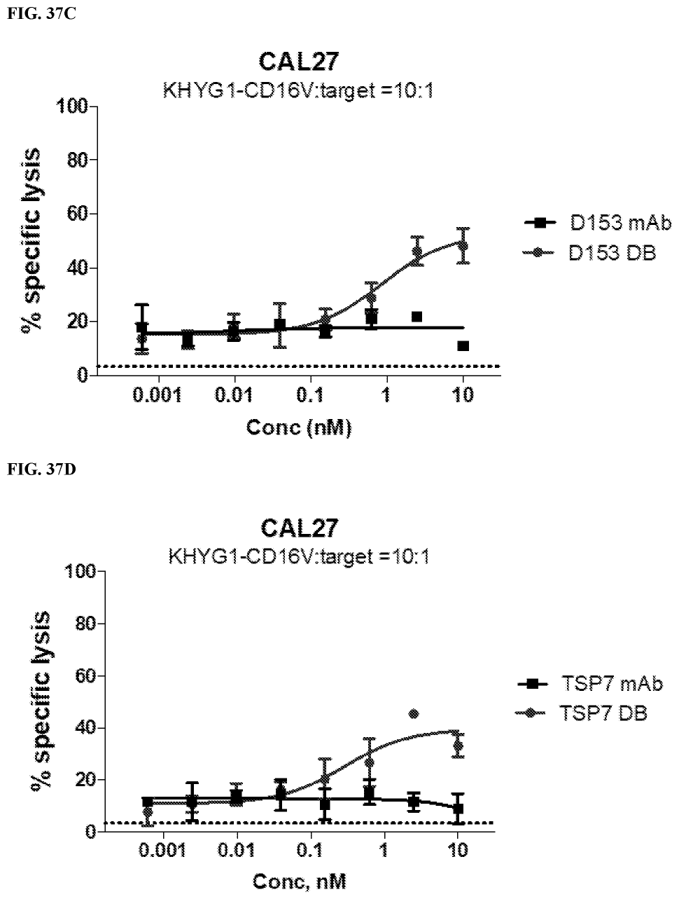

As shown in FIG. 37, anti-P-cadherin multi-specific binding proteins stimulated KHYG1-CD16V-mediated cytotoxicity against human cancer cells. FIG. 37A shows that multi-specific binding proteins having a P-cadherin (CDH3) binding site comprising a heavy chain variable domain sequence identical to SEQ ID NO:196 and a light chain variable domain sequence identical to SEQ ID NO:200 (D153 DB) stimulated KHYG1-CD16V-mediated cytotoxicity against H1975 cancer cells. Similarly, FIG. 37B shows that multi-specific binding proteins having a P-cadherin (CDH3) binding site comprising a heavy chain variable domain sequence identical to SEQ ID NO:188 and a light chain variable domain sequence identical to SEQ ID NO:192 (TSP7 DB) stimulated KHYG1-CD16V-mediated cytotoxicity against H1975 cancer cells. FIG. 37C shows that multi-specific binding proteins having a P-cadherin (CDH3) binding site comprising a heavy chain variable domain sequence identical to SEQ ID NO:196 and a light chain variable domain sequence identical to SEQ ID NO:200 (D153 DB) stimulated KHYG1-CD16V-mediated cytotoxicity against CAL27 cancer cells. Similarly, FIG. 37D shows that multi-specific binding proteins having a P-cadherin (CDH3) binding site comprising a heavy chain variable domain sequence identical to SEQ ID NO:188 and a light chain variable domain sequence identical to SEQ ID NO:192 (TSP7 DB) stimulated KHYG1-CD16V-mediated cytotoxicity against CAL27 cancer cells.

While anti-P-cadherin duobody multi-specific binding proteins stimulated target cell lysis, corresponding mAb having the same P-cadherin binding site, did not exhibit any cytotoxic activity (FIGS. 37A-D).

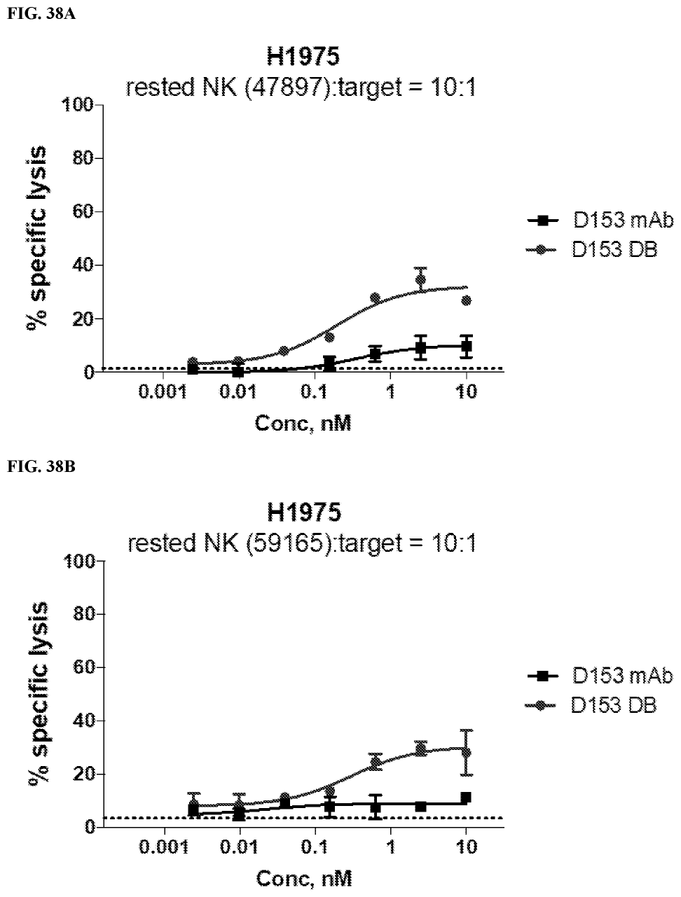

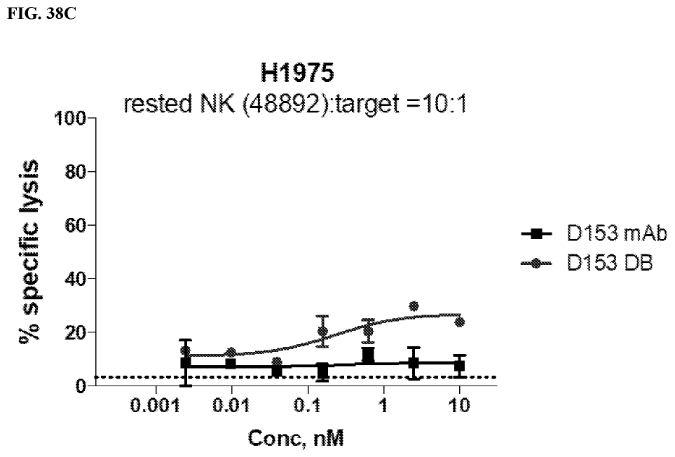

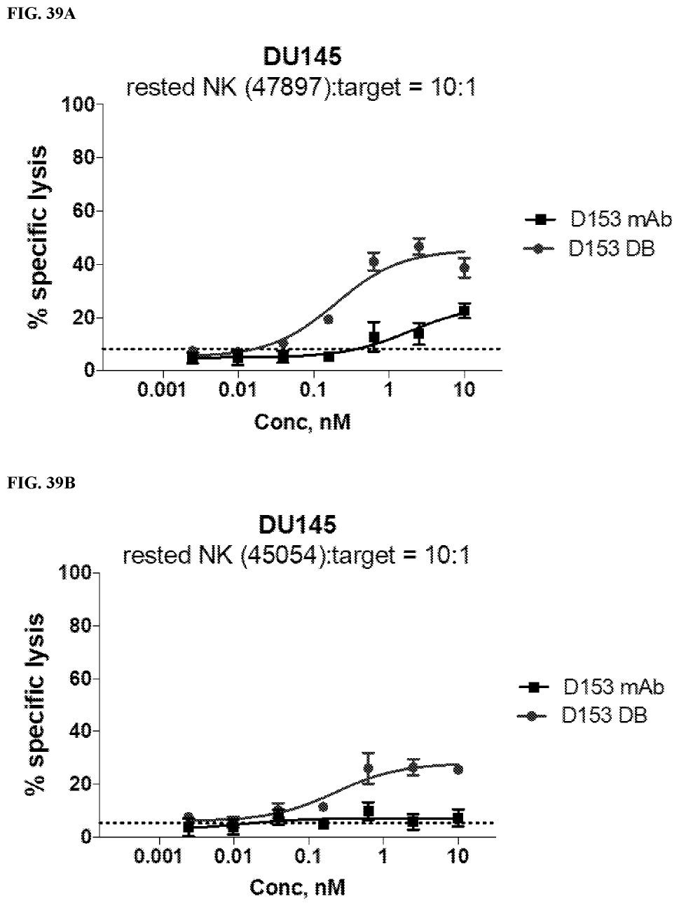

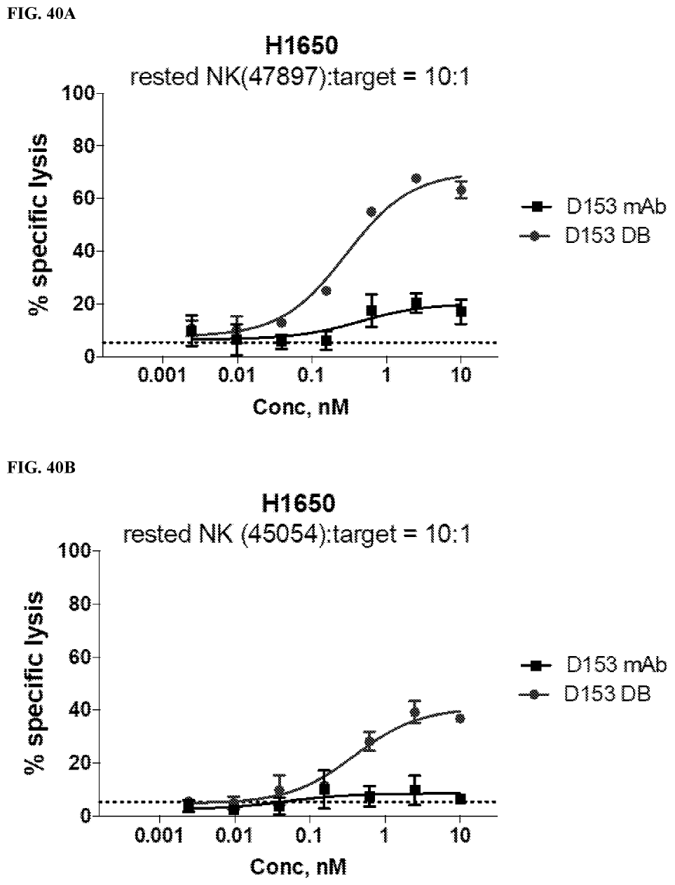

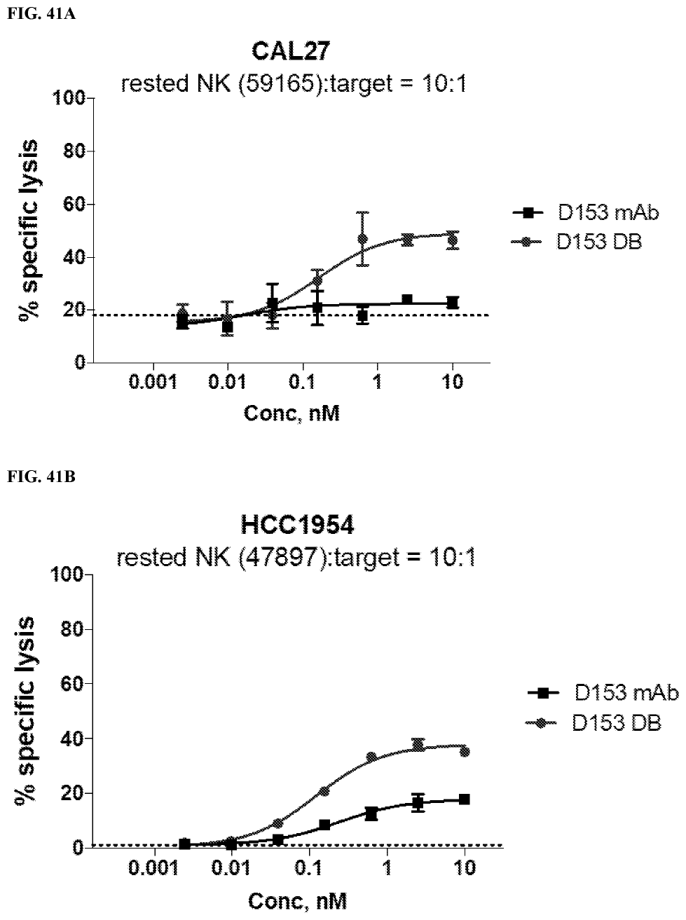

As shown in FIGS. 38, 39, 40, and 41, multi-specific binding proteins having a P-cadherin (CDH3) binding site comprising a heavy chain variable domain sequence identical to SEQ ID NO:196 and a light chain variable domain sequence identical to SEQ ID NO:200 (D153 DB) stimulated cytotoxic activity of primary human NK cells against P-cadherin-expressing human cancer cells. FIG. 38 shows that anti-P-cadherin multi-specific binding protein (D153 DB) stimulated cytotoxic activity of primary human NK cells isolated from Donor 47897 (FIG. 38A), Donor 59165 (FIG. 38B), and Donor 48892 (FIG. 38C) against P-cadherin-expressing H1975 human cancer cells. Similarly, FIG. 39 shows that anti-P-cadherin multi-specific binding protein (D153 DB) stimulated cytotoxic activity of primary human NK cells isolated from Donor 47897 (FIG. 39A) and Donor 45054 (FIG. 39B) against P-cadherin-expressing DU145 human cancer cells. FIG. 40 shows that anti-P-cadherin multi-specific binding protein (D153 DB) also stimulated cytotoxic activity of primary human NK cells isolated from Donor 47897 (FIG. 40A) and Donor 45054 (FIG. 40B) against P-cadherin-expressing H1650 human cancer cells. FIG. 41 shows that anti-P-cadherin multi-specific binding protein (D153 DB) also stimulated cytotoxic activity of primary human NK cells isolated from Donor 59165 (FIG. 41A) and Donor 47897 (FIG. 41B) against P-cadherin-expressing CAL27 and HCC1954 human cancer cells, respectively.

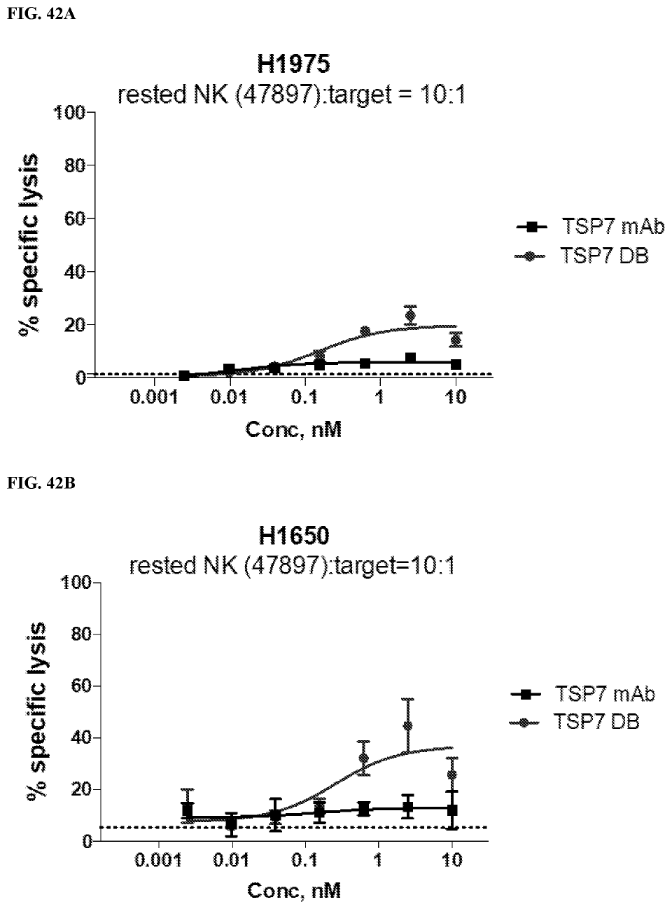

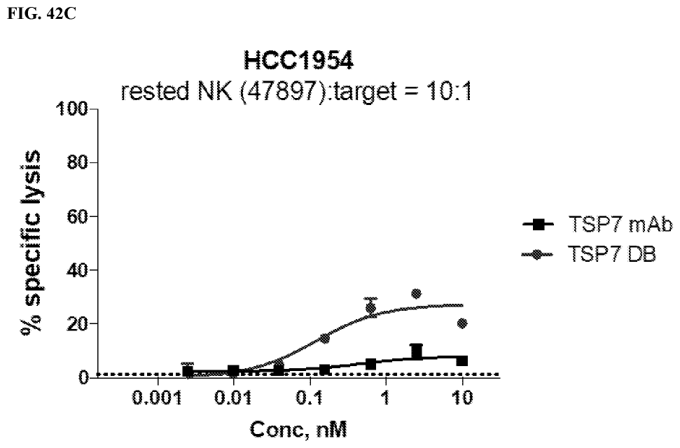

Similarly, as shown in FIGS. 42A-C, multi-specific binding protein having a P-cadherin (CDH3) binding site comprising a heavy chain variable domain sequence identical to SEQ ID NO:188 and a light chain variable domain sequence identical to SEQ ID NO:192 (TSP7 DB) stimulated cytotoxic activity of donor-purified primary human NK cells against P-cadherin expressing H1975 (FIG. 42A), H1650 (FIG. 42B), and HCC1954 (FIG. 42C) human cancer cells.

Both anti-P-cadherin multi-specific binding proteins, stimulated lysis of target cancer cells more effectively than corresponding mAbs having the same P-cadherin binding site.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A protein comprising:
(a) a first antigen-binding site that binds NKG2D;
(b) a second antigen-binding site that binds P-cadherin; and
(c) an antibody Fc domain or a portion thereof sufficient to bind CD16, or a third antigen-binding site that binds CD16.

2. The protein according to claim 1, wherein the first antigen-binding site binds to NKG2D in humans, non-human primates, and rodents.

3. The protein according to claim 1 or 2, wherein the first antigen-binding site comprises a heavy chain variable domain and a light chain variable domain.

4. The protein according to claim 3, wherein the heavy chain variable domain and the light chain variable domain are present on the same polypeptide.

5. The protein according to claim 3 or 4, wherein the second antigen-binding site comprises a heavy chain variable domain and a light chain variable domain.

6. The protein according to claim 5, wherein the heavy chain variable domain and the light chain variable domain of the second antigen-binding site are present on the same polypeptide.

7. The protein according to claim 5 or 6, wherein the light chain variable domain of the first antigen-binding site has an amino acid sequence identical to the amino acid sequence of the light chain variable domain of the second antigen-binding site.

8. A protein according to any one of claims 1 to 7, wherein the first antigen-binding site comprises a heavy chain variable domain at least 90% identical to an amino acid sequence selected from: SEQ ID NO:1, SEQ ID NO:41, SEQ ID NO:49, SEQ ID NO:57, SEQ ID NO:59, SEQ ID NO:61, SEQ ID NO:69, SEQ ID NO:77, SEQ ID NO:85, SEQ ID NO:158, SEQ ID NO:162, SEQ ID NO:166, SEQ ID NO:170, SEQ ID NO:174, SEQ ID NO:178, and SEQ ID NO:93.

9. The protein according to any one of claims 1 to 7, wherein the first antigen-binding site comprises a heavy chain variable domain at least 90% identical to SEQ ID NO:41 and a light chain variable domain at least 90% identical to SEQ ID NO:42.

10. The protein according to any one of claims 1 to 7, wherein the first antigen-binding site comprises a heavy chain variable domain at least 90% identical to SEQ ID NO:49 and a light chain variable domain at least 90% identical to SEQ ID NO:50.

11. The protein according to any one of claims 1 to 7, wherein the first antigen-binding site comprises a heavy chain variable domain at least 90% identical to SEQ ID NO:57 and a light chain variable domain at least 90% identical to SEQ ID NO:58.

12. The protein according to any one of claims 1 to 7, wherein the first antigen-binding site comprises a heavy chain variable domain at least 90% identical to SEQ ID NO:59 and a light chain variable domain at least 90% identical to SEQ ID NO:60.

13. The protein according to any one of claims 1 to 7, wherein the first antigen-binding site comprises a heavy chain variable domain at least 90% identical to SEQ ID NO:61 and a light chain variable domain at least 90% identical to SEQ ID NO:62.

14. The protein according to any one of claims 1 to 7, wherein the first antigen-binding site comprises a heavy chain variable domain at least 90% identical to SEQ ID NO:69 and a light chain variable domain at least 90% identical to SEQ ID NO:70.

15. The protein according to any one of claims 1 to 7, wherein the first antigen-binding site comprises a heavy chain variable domain at least 90% identical to SEQ ID NO:77 and a light chain variable domain at least 90% identical to SEQ ID NO:78.

16. The protein according to any one of claims 1 to 7, wherein the first antigen-binding site comprises a heavy chain variable domain at least 90% identical to SEQ ID NO:85, SEQ ID NO:158, SEQ ID NO:162, SEQ ID NO:166, SEQ ID NO:170, SEQ ID NO:174, or SEQ ID NO:178, and a light chain variable domain at least 90% identical to SEQ ID NO:86.

17. The protein according to any one of claims 1 to 7, wherein the first antigen-binding site comprises a heavy chain variable domain at least 90% identical to SEQ ID NO:93 and a light chain variable domain at least 90% identical to SEQ ID NO:94.

18. The protein according to any one of claims 1 to 7, wherein the first antigen-binding site comprises a heavy chain variable domain at least 90% identical to SEQ ID NO:101 and a light chain variable domain at least 90% identical to SEQ ID NO:102.

19. The protein according to any one of claims 1 to 7, wherein the first antigen-binding site comprises a heavy chain variable domain at least 90% identical to SEQ ID NO:103 and a light chain variable domain at least 90% identical to SEQ ID NO:104.

20. The protein according to claim 1 or 2, wherein the first antigen-binding site is a single-domain antibody.

21. The protein according to claim 20, wherein the single-domain antibody is a V_{H}H fragment or a V_{NAR} fragment.

22. The protein according to any one of claims 1 to 2 or 20 to 21, wherein the second antigen-binding site comprises a heavy chain variable domain and a light chain variable domain.

23. The protein according to claim 22, wherein the heavy chain variable domain and the light chain variable domain of the second antigen-binding site are present on the same polypeptide.

24. The protein according to any one of claims 1 to 23, wherein the heavy chain variable domain of the second antigen-binding site comprises an amino acid sequence at least 90% identical to SEQ ID NO:114 and the light chain variable domain of the second antigen-binding site comprises an amino acid sequence at least 90% identical to SEQ ID NO:118.

25. The protein according to any one of claims 1 to 23, wherein the heavy chain variable domain of the second antigen-binding site comprises an amino acid sequence at least 90% identical to SEQ ID NO:122 and the light chain variable domain of the second antigen-binding site comprises an amino acid sequence at least 90% identical to SEQ ID NO:126.

26. The protein according to any one of claims 1 to 23, wherein the heavy chain variable domain of the second antigen-binding site comprises an amino acid sequence at least 90% identical to SEQ ID NO:130 and the light chain variable domain of the second antigen-binding site comprises an amino acid sequence at least 90% identical to SEQ ID NO:134.

27. The protein according to any one of claims 1 to 23, wherein the heavy chain variable domain of the second antigen-binding site comprises an amino acid sequence at least 90% identical to SEQ ID NO:188 and the light chain variable domain of the second antigen-binding site comprises an amino acid sequence at least 90% identical to SEQ ID NO:192.

28. The protein according to any one of claims 1 to 23, wherein the heavy chain variable domain of the second antigen-binding site comprises an amino acid sequence at least 90% identical to SEQ ID NO:196 and the light chain variable domain of the second antigen-binding site comprises an amino acid sequence at least 90% identical to SEQ ID NO:200.

29. The protein according to any one of claims 1 to 23, wherein the heavy chain variable domain of the second antigen-binding site comprises an amino acid sequence at least 90% identical to SEQ ID NO:204 and the light chain variable domain of the second antigen-binding site comprises an amino acid sequence at least 90% identical to SEQ ID NO:208.

30. The protein according to any one of claims 1 to 23, wherein the heavy chain variable domain of the second antigen-binding site comprises an amino acid sequence at least 90% identical to SEQ ID NO:212 and the light chain variable domain of the second antigen-binding site comprises an amino acid sequence at least 90% identical to SEQ ID NO:216.

31. The protein according to any one of claims 1 to 23, wherein the heavy chain variable domain of the second antigen-binding site comprises an amino acid sequence at least 90% identical to SEQ ID NO:220 and the light chain variable domain of the second antigen-binding site comprises an amino acid sequence at least 90% identical to SEQ ID NO:224.

32. The protein according to any one of claims 1 to 23, wherein the heavy chain variable domain of the second antigen-binding site comprises an amino acid sequence at least 90% identical to SEQ ID NO:227 and the light chain variable domain of the second antigen-binding site comprises an amino acid sequence at least 90% identical to SEQ ID NO:229.

33. The protein according to any one of claims 1 to 23, wherein the second antigen-binding site comprises CDR1, CDR2, and CDR3 sequences of a heavy chain variable domain and a light chain variable domain sequences selected from the group consisting of SEQ ID NO: 114 and 118; 122 and 126; 130 and 134; 188 and 192; 196 and 200; 204 and 208; 212 and 216; 220 and 224; and 227 and 229, respectively.

34. The protein according to any one of claims 1 to 4 or 8 to 21, wherein the second antigen-binding site is a single-domain antibody.

35. The protein according to claim 34, wherein the second antigen-binding site is a V_{H}H fragment or a V_{NAR} fragment.

36. The protein according to any one of claims 1 to 35, wherein the protein comprises a portion of an antibody Fc domain sufficient to bind CD16, wherein the antibody Fc domain comprises hinge and CH2 domains.

37. The protein according to claim 36, wherein the antibody Fc domain comprises hinge and CH2 domains of a human IgG1 antibody.

38. The protein according to claim 36 or 37, wherein the Fc domain comprises an amino acid sequence at least 90% identical to amino acids 234-332 of a human IgG1 antibody.

39. The protein according to claim 38, wherein the Fc domain comprises amino acid sequence at least 90% identical to the Fc domain of human IgG1 and differs at one or more positions selected from the group consisting of Q347, Y349, L351, S354, E356, E357, K360, Q362, S364, T366, L368, K370, N390, K392, T394, D399, S400, D401, F405, Y407, K409, T411, K439.

40. A formulation comprising a protein according to any one of claims 1 to 39 and a pharmaceutically acceptable carrier.

41. A cell comprising one or more nucleic acids encoding a protein according to any one of claims 1 to 39.

42. A method of enhancing tumor cell death, the method comprising exposing tumor cells and natural killer cells to an effective amount of the protein according to any one of claims 1 to 39.

43. A method of treating cancer, wherein the method comprises administering an effective amount of the protein according to any one of claims 1 to 39 or the formulation according to claim 40 to a patient.

44. The method according to claim 43, wherein the second antigen binding site of the protein binds P-cadherin, the cancer to be treated is selected from the group consisting of esophageal cancer, pancreatic cancer, bladder cancer, prostate cancer, gastric cancer, lung cancer, endometrial cancer, ovarian cancer, cervical cancer, head and neck cancer, breast cancer, colorectal carcinomas, cervical adenocarcinomas, melanoma, and basocellular and squamous carcinomas of the skin.
